SEQUENCE LISTING (10 sequences)

SEQ ID NO: 1 - DNA, 23 bp, Artificial Sequence
  Primer
tcagaagcttatttaatggtgcg

SEQ ID NO: 2 - DNA, 23 bp, Artificial Sequence
  Primer
gtggttttggattcagtgagaag

SEQ ID NO: 3 - DNA, 21 bp, Artificial Sequence
  Primer
cagcttttgttgctcactacg

SEQ ID NO: 4 - DNA, 19 bp, Artificial Sequence
  Primer
tcggaacagaccttcccag

SEQ ID NO: 5 - DNA, 20 bp, Artificial Sequence
  Primer
gaggaagctgagctggtttg

SEQ ID NO: 6 - DNA, 20 bp, Artificial Sequence
  Primer
gcatcgtgctctcaattctc

SEQ ID NO: 7 - DNA, 20 bp, Artificial Sequence
  Primer
gcacgatgcatacacaggtg

SEQ ID NO: 8 - DNA, 20 bp, Artificial Sequence
  Primer
cctctgctttcagactgtct

SEQ ID NO: 9 - DNA, 20 bp, Artificial Sequence
  Primer
cggagtgttcagcagaagtc

SEQ ID NO: 10 - DNA, 20 bp, Artificial Sequence
  Primer
gaggtgacaagcacatgaac

(19) United States
(12) Patent Application Publication
(10) Pub. No.: US 2022/0087987 A1
(43) Pub. Date: Mar. 24, 2022

(54) A METHOD FOR TREATING SWI/SNF COMPLEX-DEFICIENT CANCERS COMPRISING GLUTATHIONE (GSH) METABOLIC PATHWAY INHIBITOR

(71) Applicants: National Cancer Center, Tokyo (JP); ONO PHARMACEUTICAL CO., LTD., Osaka (JP)

(72) Inventors: Hideaki OGIWARA, Tokyo (JP); Takashi KOHNO, Tokyo (JP); Hiroki SASAKI, Tokyo (JP)

(73) Assignees: National Cancer Center, Tokyo (JP); ONO PHARMACEUTICAL CO., LTD., Osaka (JP)

(21) Appl. No.: 17/418,451

(22) PCT Filed: Dec. 16, 2019
(86) PCT No.: PCT/JP2019/051342

§ 371 Date: Jun. 25, 2021

Related U.S. Application Data
(60) Provisional application No. 62/785,458, filed on Dec. 27, 2018.
(60) Provisional application No. 62/902,480, filed on Sep. 19, 2019.

(51) Int. Cl. A61K 31/439; A61K 31/197; A61K 31/4402; G01N 33/574; A61P 35/00

(52) U.S. Cl. CPC A61K 31/439; A61K 31/197; A61P 35/00; G01N 33/57484; A61K 31/4402

(57) ABSTRACT

The present invention provides a method for treating SWI/SNF complex-deficient cancers comprising a glutathione (GSH) metabolic pathway inhibitor, a pharmaceutical composition comprising a glutathione (GSH) metabolic pathway inhibitor for therapy in SWI/SNF complex-deficient cancers, and a glutathione (GSH) metabolic pathway inhibitor for use in treating SWI/SNF complex-deficient cancers. The present invention also provides a method for detecting and/or selecting a susceptible patient to a glutathione (GSH) metabolic pathway inhibitor.

### CROSS-REFERENCES TO RELATED APPLICATIONS

This application claims the benefit of the priority under 35 U.S.C. § 119(e) to U.S. Provisional Application Ser. No. 62/785,458 filed on Dec. 27, 2018 and U.S. Provisional Application Ser. No. 62/902,480 filed on Sep. 19, 2019, the disclosure of each one are incorporated herein by reference in their entirety.

### TECHNICAL FIELD

The present invention relates to a method for treating SWI/SNF complex-deficient cancers comprising a glutathione (GSH) metabolic pathway inhibitor, a pharmaceutical composition comprising a glutathione (GSH) metabolic pathway inhibitor for therapy in SWI/SNF complex-deficient cancers, and a glutathione (GSH) metabolic pathway inhibitor for use in treating SWI/SNF complex-deficient cancers.

### BACKGROUND ART

The SWI/SNF complex was originally described in yeast as the protein complex critical for cellular responses to mating-type switching (SWI) or sucrose fermentation (SNF). Recently, the SWI/SNF complex has been linked to a number of human diseases, most notably, cancer. Loss-of-function mutations of genes encoding subunits of the SWI/SNF chromatin-remodeling complex are found in approximately 20% of all human cancers. Such mutations are thought to promote tumorigenesis by disturbing transcriptional homeostasis due to impairment of chromatin remodeling for transcription, DNA damage repair, DNA replication and chromatin segregation. The ARID1A gene, which encodes a component of the SWI/SNF chromatin-remodeling complex, is frequently mutated in several intractable cancers; it is mutated in about 46% of ovarian clear cell carcinoma (OCCC), 33% of gastric carcinoma, 27% of cholangiocarcinoma and 15% of pancreatic carcinoma [see, NPL 1-4], all of which lack effective molecular targeting therapies. Furthermore, ARID1A deficiency is associated with poor prognosis of a variety of cancers [see, NPL 5]. Thus, much effort has been devoted to identifying vulnerabilities caused by ARID1A deficiency for developing effective therapeutic modalities against those intractable cancers [see, NPL 6].

Similarly, the other components of the SWI/SNF complex, such as SMARCA2 (BRM), SMARCA4 (BRG1), SMARCB1, PBRM1, are also known to be mutated in the various cancers [see, NPL 7].

Regulation of oxidative stress homeostasis is important for cell survival. Reactive oxygen species (ROS) cause oxidative stress. Cellular ROS levels are determined by the balance between ROS generation and elimination, and are regulated by antioxidant defense mechanisms. High levels of ROS cause cell damage and death. Therefore, targeting antioxidant defense systems may be an anti-cancer therapeutic strategy. Glutathione (GSH) is an abundant antioxidant tripeptide molecule synthesized from cysteine, glutamate, and glycine by the ATP-dependent enzyme glutamate-cysteine ligase synthetase (GCL), which is composed of the glutamate-cysteine ligase catalytic subunit (GCLC) and the glutamate-cysteine ligase modifier subunit (GCLM), and GSH synthetase (GSS) [see, NPL 8]. GCL catalyzes the rate-limiting step of glutamate ligation with cysteine during GSH synthesis. Cysteine is the rate-limiting precursor substrate for GSH synthesis. Intracellular cysteine levels are controlled by SLC7A11, which encodes the cystine/glutamate transporter XCT.

Conventional molecular target drugs can selectively inhibit gene mutations which activate prolifiration of cancer cells, but many patients in which gene mutations cannot be detected are not targets of the molecular target drugs. However, molecular target drugs cannot be applied to loss-of-function mutations of genes. Therefore, a new therapy has been required for loss-of-function mutations of genes.

Some loss-of-function mutations of genes confer druggable vulnerabilities on cancer cells. “Synthetic lethality,” which is defined by an interdependent relationship between two genes, which means that simultaneous loss of two genes, but not loss of either gene alone, leads to cell death. Therefore, cancer cells harboring such a deleterious gene mutation would be vulnerable to inhibition of the synthetic lethal target, as epitomized by the success of PARP1-targeted therapy against hereditary breast and ovarian cancers harboring BRCA1 and BRCA2 mutations [see, NPL 9].

However, no efficient therapeutic strategy in SWI/SNF complex-deficient cancers has been established. The goal of the invention is to develop effective therapeutic strategy for treating SWI/SNF complex-deficient cancers by identifying druggable targets.

### Non-Patent Literatures

[NPL 1] N Engl J Med 363 (2010) 1532-1543
[NPL 2] Science 330 (2010) 228-231
[NPL 3] Nature 505 (2014) 495-501
[NPL 4] Cell 173 (2018) 305-320
[NPL 5] Oncotarget 6 (2015) 39088-39097
[NPL 6] Oncotarget 7 (2016) 56933-56943
[NPL 7] Cancer Letters 419 (2018) 266-279
[NPL 8] Biomed. Res. Int. 2017, Article ID 9584932
[NPL 9] Lord and Ashworth, 2016

### SUMMARY OF INVENTION

It has unexpectedly been discovered that since the expression of SCL7A11 protein which carries cystein, an element constituting Glutathione (GSH), is suppressed in SWI/SNF complex-deficient cancer, the level of GSH is also lowered (referred to as “metabolome aberration due to loss-of-function”). This phenomenon cannot be found in normal cells.

In addition, when GSH itself or GSH synthesis enzymes are inhibited, the level of GSH is easily reduced, and then ROS is in excess, which induces apoptosis of SWI/SNF complex-deficient cancer cells. We have discovered that various cancers having SWI/SNF complex gene mutations can be treated using inhibitors of GSH or GSH synthesis enzymes (“synthetic lethal targets”). This new therapy is referred to as a “synthetic lethality therapy.” Specifically, in the first embodiment, the present invention provides a method for treating SWI/SNF complex-deficient cancer, comprising administering an effective amount of a glutathione (GSH) metabolic pathway inhibitor to a mammal in need thereof.

In the second embodiment, the present invention provides a method for detecting and/or selecting a susceptible patient to a glutathione (GSH) metabolic pathway inhibitor, comprising detecting the presence of SWI/SNF complex protein deficiency in a patient having cancer.

In the third embodiment, the present invention provides a pharmaceutical composition comprising an effective amount of a glutathione (GSH) metabolic pathway inhibitor for treating SWI/SNF complex-deficient cancer.

In the fourth embodiment, the present invention provides a glutathione (GSH) metabolic pathway inhibitor for use in treating SWI/SNF complex-deficient cancer.

In the fifth embodiment, the present invention provides a kit comprising (a) a glutathione (GSH) metabolic pathway inhibitor, and (b) instructions for treating SWI/SNF complex-deficient cancer.

### BRIEF DESCRIPTION OF DRAWINGS

FIG. 1-1 shows that ARID1A-deficient cancer cells are selectively sensitive to PRIMA-1 and APR-246. (A) shows immunoblotting for ARID1A and β-actin in whole-cell extracts of ARID1A-WT and ARID1A-KO HCT116 cells. (B) shows scheme showing screening for compounds that selectively suppress the growth of ARID1A-KO cells. (C) shows heat map of the cell viabilities of ARID1A-WT and ARID1A-KO HCT116 cells after treatment with 334 compounds (each at 10 μM) for 5 days. A red arrow indicates results from PRIMA-1-treated samples.

FIG. 1-2 shows that ARID1A-deficient cancer cells are selectively sensitive to PRIMA-1 and APR-246. (D) shows cell viabilities of ARID1A-WT and ARID1A-KO HCT116 cells after treatment for 5 days with 3.7 μM PRIMA-1. (E) shows colony formation of ARID1A-WT and ARID1A-KO HCT116 cells after treatment for 10 days with 2.5 μM PRIMA-1. (F) shows cell viabilities of ARID1A-WT and ARID1A-KO HCT116 cells after treatment for 5 days with 3.7 μM APR-246. (G) shows colony formation of ARID1A-WT and ARID1A-KO HCT116 cells after treatment for 10 days with 2 μM APR-246.

FIG. 1-3 shows that ARID1A-deficient cancer cells are selectively sensitive to PRIMA-1 and APR-246. (H) shows immunoblotting for ARID1A and 3-actin in whole-cell extracts of the indicated cell lines.

FIG. 1-4 shows that ARID1A-deficient cancer cells are selectively sensitive to PRIMA-1 and APR-246. (I) shows cell viabilities of cancer cell lines after treatment for 3 days with 11 μM APR-246. (J) shows colony formation of ovarian cancer cell lines after treatment for 14 days with 1 μM APR-246. (K) shows immunoblotting for ARID1A and 3-actin in whole-cell extracts of ARID1A-deficient TOV21G cancer cells with or without ectopic expression of ARID1A cDNA (left) and cell viabilities of these cells after treatment for 3 days with 11 μM APR-246 (right).

FIG. 1-5 shows that ARID1A-deficient cancer cells are selectively sensitive to PRIMA-1 and APR-246. (L) shows a Venn diagram showing the number of genes in ARID1A-WT and ARID1A-KO HCT116 cells with altered expression after treatment for 24 hrs with 40 μM APR-246 and the top five most enriched pathways among 6,429 genes with significantly altered expression in ARID1A-KO cells by WikiPathways database analysis.

FIG. 1-6 shows that ARID1A-deficient cancer cells are selectively sensitive to PRIMA-1 and APR-246. (M) shows gene expression profiles of two apoptosis-related pathways in ARID1A-WT and ARID1A-KO HCT116 cells after treatment for 24 hrs with 40 μM APR-246. Red arrow heads indicate the profile of the NOXA gene.

FIG. 1-7 shows that ARID1A-deficient cancer cells are selectively sensitive to PRIMA-1 and APR-246. (N) shows relative mRNA levels of NOXA in ARID1A-WT and ARID1A-KO HCT116 cells after treatment for 24 hrs with 20 μM APR-246. (O) shows immunoblotting for ARID1A, NOXA and 3-actin in whole-cell extracts of ARID1A-WT and ARID1A-KO HCT116 cells after treatment for 24 hrs with 40 μM APR-246. (P) shows detection of cleaved caspase-3/7-positive apoptotic cells in ARID1A-WT and ARID1A-KO HCT116 cells after treatment for 48 hrs with 10 μM APR-246. (Q) shows detection of Annexin V-positive apoptotic cells in ARID1A-WT and ARID1A-KO HCT116 cells after treatment for 48 hrs with 15 μM APR-246. (R) shows detection of Annexin V-positive apoptotic cells in ovarian cancer cell lines after treatment for 48 hrs with 50 μM APR-246. Data in (D, F, I, K, N, P, Q, and R) are expressed as the mean±SD. See also FIG. S1 and Table S1. Table S1 shows Cell viabilities of HCT116 ARID1A-WT and ARID1A-KO in treatment with 334 compounds at 10 μM for 5 days. Related to FIGS. 1-1 to 1-7.

FIG. 2-1 shows that ARID1A-deficient cells are vulnerable to GSH inhibition. A) shows schematic of the candidate targets of APR-246. (B) to (D) show relative GSH levels (B), relative TrxR activities (C), and relative ROS levels (D) in ARID1A-WT and ARID1A-KO HCT116 cells after treatment with APR-246 for 24, 24, and 48 hrs, respectively.

FIG. 2-2 shows that ARID1A-deficient cells are vulnerable to GSH inhibition. (E) shows heatmap of the ratios of the signal intensities in ARID1A-WT and ARID1A-KO HCT116 cells treated with 40 μM APR-246 for 24 hrs relative to the corresponding intensities in untreated cells. (F) shows immunoblotting for JNK, phospho-JNK (pJNK), and β-actin in whole-cell extracts of ARID1A-KO HCT116 cells after treatment with 25 or 50 μM APR-246 for 24 hrs. The red arrow head indicates the phosphorylated form of JNK. (G) to FIG. 2-3 (H) show relative GSH levels (G) and relative ROS levels (H) in RMG-I, TOV21G and OVISE cells after treatment with 5 μM APR-246 for 16 and 48 hrs, respectively.

FIG. 2-3 shows that ARID1A-deficient cells are vulnerable to GSH inhibition. (I) to FIG. 2-3 (J) and FIG. 2-4 (K) show relative GSH levels (I), relative ROS levels (J), and cell viabilities (K) in ARID1A-deficient TOV21G cancer cells with or without ectopic expression of ARID1A cDNA after treatment with 30 μM APR-246 for 24, 48, and 48 hrs, respectively.

FIG. 2-4 shows that ARID1A-deficient cells are vulnerable to GSH inhibition. (L) to FIG. 2-4 (M) and FIG. 2-5 (N) to FIG. 2-5 (O) show that relative GSH levels (L), relative ROS levels (M), cell viabilities (N), and levels of apoptosis (O) in ARID1A-KO HCT116 cells after treatment with 40 μM APR-246 for 24, 48, 48, and 48 hrs, respectively, without or with co-treatment with 5 mM NAC or 5 mM GSH-MEE. Data in (B-D, G-H and I-O) are expressed as the mean±SD. See also FIGS. S2-1 to S2-7.

FIG. 3-1 shows that GCLC is a promising synthetic lethal target for ARID1A-deficient cancers. (A) shows schematic of molecules involved in the GSH metabolic pathway and inhibitors of their products.

FIG. 3-2 shows that GCLC is a promising synthetic lethal target for ARID1A-deficient cancers. (B) to (D) show heat map of cell viabilities (B), relative GSH levels (C), and relative ROS levels (D) in ARID1A-proficient RMG-I cells and ARID1A-deficient TOV21G cells at 5, 3, and 3 days, respectively, after knockdown of GSH pathway genes.

FIG. 3-3 shows that GCLC is a promising synthetic lethal target for ARID1A-deficient cancers. (E) shows cell viabilities of ARID1A-proficient RMG-I cells and ARID1A-deficient TOV21G cells after treatment with BSO, sulfasalazine, compound 968, or 6-aminonicotinamide for 3 days.

FIG. 3-4 shows that GCLC is a promising synthetic lethal target for ARID1A-deficient cancers. (F) to (I) show relative GCLC mRNA levels (F), cell viabilities (G), relative GSH levels (H), and relative ROS levels (I) in ARID1A-proficient 2008 cells and ARID1A-deficient TOV21G cells at 3 days after shRNA-mediated knockdown of GCLC by treatment with 0.5 μg/mL doxycycline.

FIG. 3-5 shows that GCLC is a promising synthetic lethal target for ARID1A-deficient cancers. (J) shows colony formation of 2008 and TOV21G cells at 14 days after knockdown of GCLC genes. (K) to FIG. 3-5 (N) show immunoblotting for GCLC and 3-actin in whole-cell extracts (K), relative GSH levels (L; 3 days), relative ROS levels (M; 3 days), and cell viability (N; 7 days) in parental TOV21G cells and TOV21G cells stably expressing GCLC after knockdown of GCLC.

FIG. 3-6 shows that GCLC is a promising synthetic lethal target for ARID1A-deficient cancers. (O) shows cell viabilities of cancer cell lines after treatment with 33 μM BSO for 3 days. (P) shows colony formation of 2008, TOV21G, and OVISE cancer cells at 14 days after treatment with 10 μM BSO. (Q) shows detection of Annexin V-positive apoptotic cells in 2008, TOV21G, and OVISE cells after treatment with 200 μM BSO for 72 hr.

FIG. 3-7 shows that GCLC is a promising synthetic lethal target for ARID1A-deficient cancers. (R) to (U) show immunoblotting for GCLC and 3-actin in whole-cell extracts (R), relative GSH levels (S; 1 day), relative ROS levels (T; 1 day), and cell viabilities (U; 2 days) in parental TOV21G cells and TOV21G cells stably expressing GCLC after treatment with 100 μM BSO. Data in (E-I, L-O, Q and S-U) are expressed as the mean±SD. See also FIGS. S3-1 to S3-5 and FIGS. S4-1 to S4-6.

FIG. 4-1 shows that vulnerability of ARID1A-deficient cancer cells to GSH inhibition is caused by decreased GSH synthesis due to impaired SLC7A11 expression. (A) shows a Venn diagram showing the numbers of significantly downregulated (fold change<−1.5) genes in ARID1A-deficient cells compared to ARID1A-proficient cells. (B) shows relative expressions of SLC7A11 mRNA (left) and protein (right) in ARID1A-proficient RMG-I cells and ARID1A-deficient TOV21G cells. (C) shows relative expressions of SLC7A11 mRNA (left) and protein (right) in parental TOV21G cells and TOV21G cells stably expressing ARID1A.

FIG. 4-2 shows that vulnerability of ARID1A-deficient cancer cells to GSH inhibition is caused by decreased GSH synthesis due to impaired SLC7A11 expression. (D) shows relative expressions of SLC7A11 mRNA (left) and protein (right) in ARID1A-WT and ARID1A-KO HCT116 cells. (E) to (F) and FIG. 4-3 (G) to FIG. 4-3 (H) show ChIP analysis of the localization of ARID1A (E), BRG1 (F), RNAPII (G), and NRF2 (H) around the TSS of SLC7A11 in ARID1A-WT and ARID1A-KO HCT116 cells.

FIG. 4-3 shows that vulnerability of ARID1A-deficient cancer cells to GSH inhibition is caused by decreased GSH synthesis due to impaired SLC7A11 expression. (I) shows schematic of the mechanism underlying regulation of SLC7A11 expression by ARID1A.

FIG. 4-4 shows that vulnerability of ARID1A-deficient cancer cells to GSH inhibition is caused by decreased GSH synthesis due to impaired SLC7A11 expression. (J) shows a Venn diagram showing metabolites downregulated (fold change<−2) in ARID1A-deficient cells compared to ARID1A-proficient cells. (K) shows basal cysteine levels in ARID1A-proficient RMG-I cells and ARID1A-deficient TOV21G cells. Data are expressed as the mean±SEM (n=3). *p<0.05; two-tailed t-test. (L) shows basal GSH levels in ARID1A-proficient RMG-I cells and ARID1A-deficient TOV21G cells.

FIG. 4-5 shows that vulnerability of ARID1A-deficient cancer cells to GSH inhibition is caused by decreased GSH synthesis due to impaired SLC7A11 expression. (M) shows schematic of the GSH synthesis pathway. (N) shows basal GSH levels in parental TOV21G cells and TOV21G cells stably expressing ARID1A. (O) to (P) show immunoblotting for SLC7A11 and β-actin (O) and the basal GSH level (P) in parental TOV21G cells and TOV21G cells stably expressing SLC7A11. (Q) to (R) and FIG. 4-6 (S) show relative GSH levels (Q), relative ROS levels (R), and cell viabilities (S) in parental TOV21G cells and TOV21G cells stably expressing SLC7A11 after treatment with 20 μM APR-246 for 24 hrs.

FIG. 4-6 shows that vulnerability of ARID1A-deficient cancer cells to GSH inhibition is caused by decreased GSH synthesis due to impaired SLC7A11 expression. (T) to (V) show relative GSH levels (T), relative ROS levels (U), and cell viabilities (V) in ARID1A-deficient TOV21G cells after treatment with 30 μM APR-246 for 24 and 48 hrs, respectively, without or with co-treatment with 100 μM CC-DME.

FIG. 4-7 shows that vulnerability of ARID1A-deficient cancer cells to GSH inhibition is caused by decreased GSH synthesis due to impaired SLC7A11 expression. (W) shows schematic model explaining the vulnerability of ARID1A-deficient cancers to inhibition of GSH metabolism. The signs of (in)equality indicate the balance between GSH activity and ROS. Data in (B-H, L, N, and P-V) are expressed as the mean±SD. See also FIGS. S5-1 to S5-7.

FIG. 5-1 shows clinical relevance of GSH-targeting therapy for ARID1A-deficient cancers. (A) and FIG. 5-2 (B) show immunohistochemical staining for ARID1A and SLC7A11 in ovarian cancer specimens with wild-type (A) or mutant (B) ARID1A. Scale bar, 50 μm.

FIG. 5-3 shows clinical relevance of GSH-targeting therapy for ARID1A-deficient cancers. (C) shows immunoblotting for ARID1A and β-actin in whole-cell extracts of ovarian PDCs. (D) shows cell viabilities of ovarian PDCs after treatment with 33 μM APR-246 for 6 days. (E) to FIG. 5-4 (F) show relative expression of SLC7A11 mRNA (E) and protein (F) in ovarian PDCs.

FIG. 5-4 shows clinical relevance of GSH-targeting therapy for ARID1A-deficient cancers. (G) shows basal GSH levels in ovarian PDCs. (H) to FIG. 5-5 (I) show relative GSH levels (H) and relative ROS levels (I) in ovarian PDCs after treatment with 50 μM APR-246 for 24 hrs.

FIG. 5-5 shows clinical relevance of GSH-targeting therapy for ARID1A-deficient cancers. (J) shows detection of Annexin V-positive apoptotic cells in ovarian PDCs after treatment with 40 μM APR-246 for 48 hrs. Data in (D-E and G-J) are expressed as the mean±SD. See also FIGS. S6-1 to S6-6.

FIG. 6-1 shows anti-tumor effect of GSH-targeting therapy for ARID1A-deficient cancers. (A) to (D) show tumor volume (A, B) and relative tumor weights (C, D) of xenografts derived from ARID1A-proficient RMG-I (A, C) or ARID1A-deficient TOV21G (B, D) cells in mice treated with 50 mg/kg APR-246 (n=6). (E) shows relative GSH ratio in xenografts derived from ARID1A-deficient TOV21G cells in mice treated with 50 mg/kg APR-246 (n=6).

FIG. 6-2 shows anti-tumor effect of GSH-targeting therapy for ARID1A-deficient cancers. (F) shows immunohistochemical (IHC) staining for 8-OHdG, NOXA, cleaved caspase-3, cleaved PARP; and Ki67 in xenografts derived from ARID1A-deficient TOV21G cells in mice treated with 50 mg/kg APR-246. Scale bar, 50 μm. (G) shows histological score (H-score) of IHC staining for 8-OHdG in xenografts derived from ARID1A-deficient TOV21G cells in mice treated with 50 mg/kg APR-246 (n=6). (H) to FIG. 6-3 (K) show percentage of cells staining positive for NOXA (H), cleaved caspase-3 (I), cleaved PARP (J), and Ki67 (K) in tumor of xenografts derived from ARID1A-deficient TOV21G cells in mice treated with 50 mg/kg APR-246 (n=6).

FIG. 6-3 shows anti-tumor effect of GSH-targeting therapy for ARID1A-deficient cancers. (L) to FIG. 6-4 (O) show tumor volume (L, M) and relative tumor weight (N, O) of xenografts derived from TOV21G-shNT (L, N) and TOV21G-shGCLC (M, O) cells in mice treated without or with doxycycline (Dox). After tumor formation, mice were fed a diet with or without Dox (n=6).

FIG. 6-4 shows anti-tumor effect of GSH-targeting therapy for ARID1A-deficient cancers. (P) shows relative GSH levels in tumor of xenografts derived from TOV21G-shNT and TOV21G-shGCLC cells in mice treated without or with Dox (n=6). Data in (A-E and G-P) are expressed as the mean±SEM. *p<0.05, **p<0.01, ***p<0.001; two-tailed t-test. See also FIGS. S6-1 to S6-6.

FIG. 7 shows ARID1A protein expression in diffuse-type gastric cancer patient-derived cell (PDC) lines and histology of xenograft tumors. (A) shows immunoblotting for ARID1A, SLC7A11, and β-actin in whole-cell extracts of diffuse-type gastric cancer PDCs. (B) shows hematoxylin and eosin staining of xenograft tumors derived from PDCs. NSC-64C, NSC-70C, and NSC-7C tumors showed poorly differentiated histology. NSC-65C tumors showed poorly or moderately differentiated histology. Scale bar, 50 μm.

FIG. 8 shows that ARID1A-deficient diffuse-type gastric cancer PDCs are sensitive to GSH inhibitors. (A) shows IC50 values for APR-246 in DGC PDCs including four ARID1A-WT PDCs and ARID1A-deficient PDCs after treatment for 6 days. Data are expressed as the mean±SEM. (n=4) *p<0.05; two-tailed t-test. (B) shows relative area under the curve (AUC) values for cell viability in PDCs including four ARID1A-WT PDCs and ARID1A-deficient PDCs treated with APR-246 for 6 days. Data are expressed as the mean±SEM. (n=4) *p<0.05; two-tailed t-test. (C) shows relative AUC values for cell viability in PDCs including four ARID1A-WT PDCs and ARID1A-deficient PDCs treated with BSO for 6 days. Data are expressed as the mean±SEM. (n=4) *p<0.05; two-tailed t-test.

FIG. 9 shows that ARID1A-deficient gastric cancer cells are vulnerable to GSH inhibition due to low basal levels of GSH. (A) shows relative expression of SLC7A11 mRNA in PDCs: ARID1A-WT NSC-48CA cells and ARID1A-deficient NSC-67C cells. Data are expressed as the mean±SD. (B) shows basal GSH levels in PDCs: ARID1A-WT NSC-48CA cells and ARID1A-deficient NSC-67C cells. Data are expressed as the mean±SD. (C) shows relative levels of GSH/GSSG, which indicates the ratio of reduced GSH to the oxidized form GSH disulfide (GSSG), in ARID1A-WT NSC-48CA cells and ARID1A-deficient NSC-67C cells treated with 40 μM APR-246 for 24 hrs. Data are expressed as the mean±SD. (D) shows relative ROS levels in ARID1A-WT NSC-48CA cells and ARID1A-deficient NSC-67C cells treated with 40 μM APR-246 for 48 hrs. Data are expressed as the mean±SD.

FIG. 10 shows that vulnerability of ARID1A-deficient gastric cancer cells to GSH inhibition is caused by decreased GSH synthesis due to diminished SLC7A11 expression. (A) to (C) show relative GSH levels (A), relative ROS levels (B), and cell viabilities (C) in ARID1A-deficient NSC-67C cells after treatment with 20 μM APR-246 for 24 hrs with or without 100 μM CC-DME or 2.5 mM GSH-MEE co-treatment.

FIG. 11 shows that ARID1A-deficient uterine cancer cell lines are sensitive to GSH inhibitors. IC50 values for APR-246 in uterine cancer cell lines including four ARID1A-WT uterine cancer cell lines and ARID1A-deficient uterine cancer cell lines after treatment for 6 days.

FIG. 12 shows that ARID1A-deficient uterine cancer cell lines are sensitive to GSH inhibitors. IC50 values for BSO in uterine cancer cell lines including four ARID1A-WT uterine cancer cell lines and ARID1A-deficient uterine cancer cell lines after treatment for 6 days.

FIG. 13 shows that ARID1A-deficient bile duct cancer cell lines are sensitive to GSH inhibitors. IC50 values for APR-246 in bile duct cancer cell lines including four ARID1A-WT uterine cancer cell lines and ARID1A-deficient bile duct cancer cell lines after treatment for 6 days.

FIG. 14 shows that ARID1A-deficient bile duct cancer cell lines are sensitive to GSH inhibitors. IC50 values for BSO in bile duct cancer cell lines including four ARID1A-WT uterine cancer cell lines and ARID1A-deficient bile duct cancer cell lines after treatment for 6 days.

FIG. 15 shows SWI/SNF complex (SMARCA4 (BRG1), SMARCA2 (BRM), SMARCB1, PBRM1, ARID1A) proteins expression in various cancer cell lines. Immunoblotting for SMARCA4 (BRG1), SMARCA2 (BRM), SMARCB1, PBRM1, ARID1A, and 3-actin in whole-cell extracts of various cancer cell lines.

FIG. 16 shows SWI/SNF complex (SMARCA4 (BRG1), SMARCA2 (BRM), SMARCB1, PBRM1, ARID1A) proteins expression in various cancer cell lines. Immunoblotting for SMARCA4 (BRG1), SMARCA2 (BRM), SMARCB1, PBRM1, ARID1A, and 3-actin in whole-cell extracts of various cancer cell lines.

FIG. 17 shows SWI/SNF complex (SMARCA4 (BRG1), SMARCA2 (BRM), SMARCB1, PBRM1) -deficient various cancer cell lines are sensitive to APR-246. IC50 values for APR-246 in various cancer cell lines including SWI/SNF complex WT cancer cell lines and SWI/SNF complex (SMARCA4 (BRG1), SMARCA2 (BRM), SMARCB1, PBRM1) -deficient various cancer cell lines after treatment for 6 days. Data are expressed as the mean±SEM. *p<0.05; two-tailed t-test.

FIG. 18 shows that SWI/SNF complex (SMARCA4 (BRG1), SMARCA2 (BRM), SMARCB1, PBRM1) -deficient various cancer cell lines are sensitive to BSO. Relative area under the curve (AUC) values for cell viability in cancer cell lines including four SWI/SNF complex WT cancer cell lines and SWI/SNF complex (SMARCA4 (BRG1), SMARCA2 (BRM), SMARCB1, PBRM1) -deficient various cancer cell lines treated with BSO for 6 days. Data are expressed as the mean±SEM. *p<0.05; two-tailed t-test.

FIG. 19 shows relative expression of SLC7A11 mRNA in various cancer cell lines: SWI/SNF complex WT cells and SWI/SNF complex (SMARCA4 (BRG1), SMARCA2 (BRM), SMARCB1, PBRM1) -deficient various cancer cell lines. Data are expressed as the mean±SD.

FIG. 20 shows basal GSH levels in various cancer cell lines: SWI/SNF complex WT cells and SWI/SNF complex (SMARCA4 (BRG1), SMARCA2 (BRM), SMARCB1, PBRM1) -deficient various cancer cell lines. Data are expressed as the mean±SD.

FIG. 21 shows relative levels of GSH/GSSG, which indicates the ratio of reduced GSH to the oxidized form GSH disulfide (GSSG), in SWI/SNF complex WT cells and SWI/SNF complex (SMARCA4 (BRG1), SMARCA2 (BRM), SMARCB1, PBRM1) -deficient various cancer cell lines treated with 40 μM APR-246 for 24 hrs. Data are expressed as the mean±SD.

FIG. 22 shows relative ROS levels in SWI/SNF complex WT cells and SWI/SNF complex (SMARCA4 (BRG1), SMARCA2 (BRM), SMARCB1, PBRM1) -deficient various cancer cell lines treated with 40 μM APR-246 for 24 hrs. Data are expressed as the mean±SD.

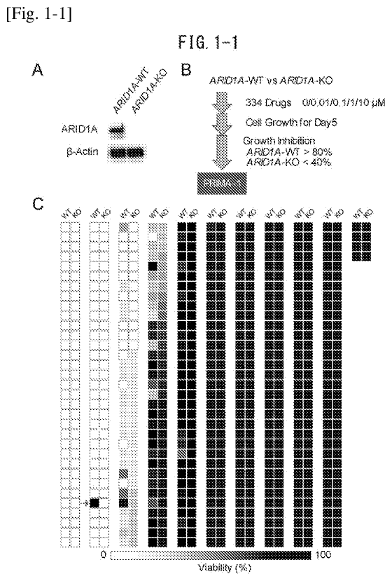

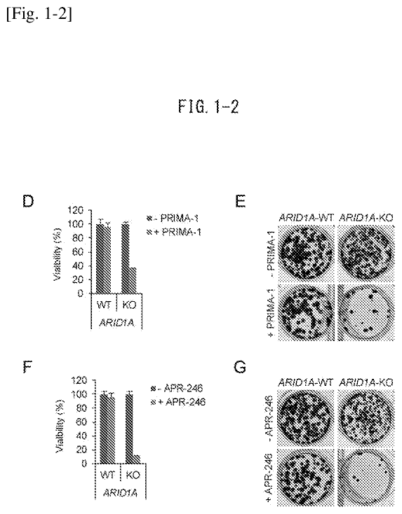

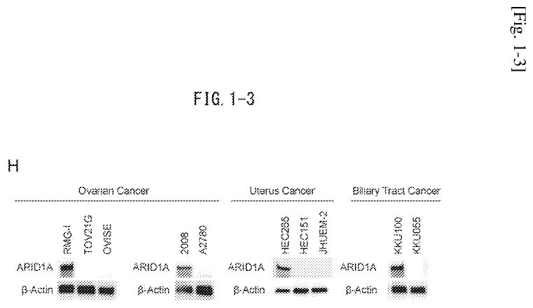

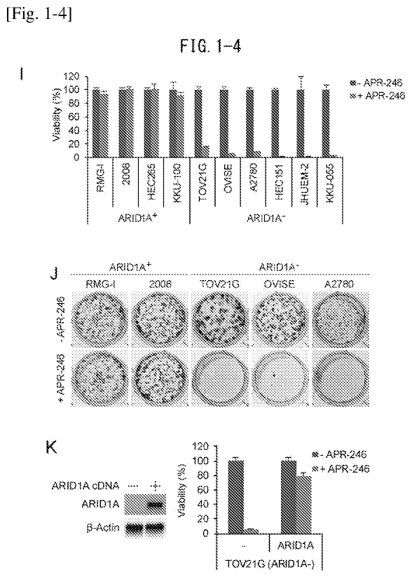

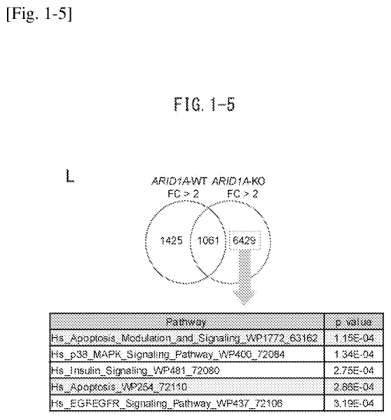

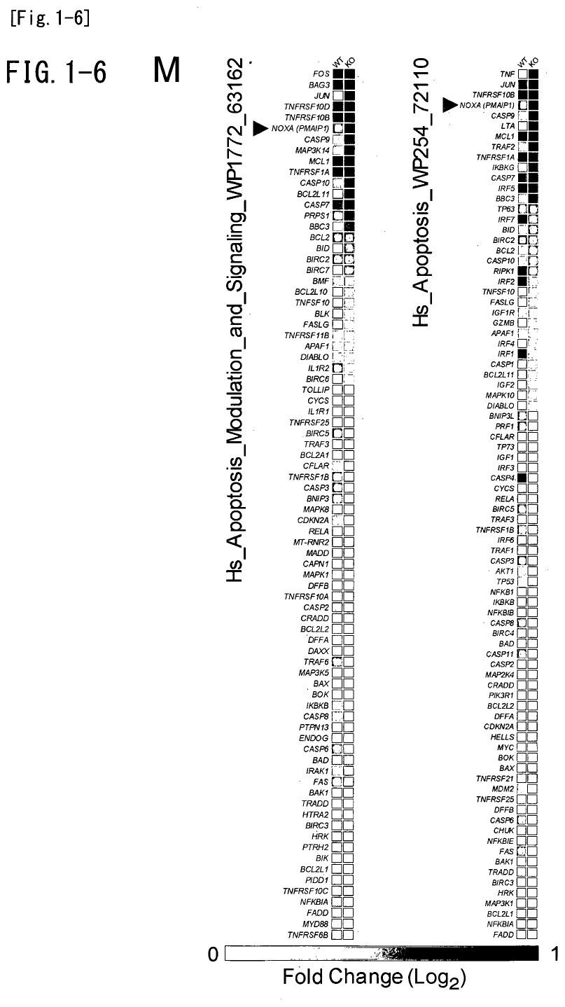

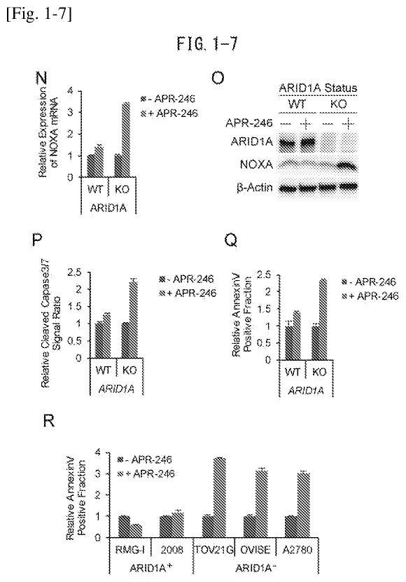

FIG. S1-1 shows that ARID1A-deficient cancer cells are selectively sensitive to PRIMA-1 and APR-246. Related to FIGS. 1-1 to 1-7. (A) shows colony formation of ARID1A-WT and ARID1A-KO HCT116 cells after treatment with 2.5 μM PRIMA-1 for 10 days. (B) shows colony formation of ARID1A-WT and ARID1A-KO HCT116 cells after treatment with 2 μM APR-246 for 10 days. (C) to (D) and Figure S1-2 (E) show cell viabilities of cancer cell lines to APR-246, including ovarian (C), uterus (D) and biliary tract (E) cancer cells.

FIG. S1-2 shows that ARID1A-deficient cancer cells are selectively sensitive to PRIMA-1 and APR-246. Related to FIGS. 1-1 to 1-7. (F) shows colony formation of ovarian cancer cell lines after treatment with 1 μM APR-246 for 14 days. (G) shows cell viabilities of ovarian cancer cells after treatment with 11 μM PRIMA-1 for 3 days.

FIG. S1-3 shows that ARID1A-deficient cancer cells are selectively sensitive to PRIMA-1 and APR-246. Related to FIGS. 1-1 to 1-7. (H) shows colony formation assays of ovarian cancer cell lines after treatment with 2.5 μM APR-246 for 14 days. (I) shows cell viabilities of parental TOV21G cells and TOV21G cells stably expressing ARID1A after treatment with 3.7 μM APR-246 for 3 days. (J) shows relative mRNA levels of NOXA in RMG-I, TOV21G and OVISE cells after treatment with 30 μM APR-246 for 24 hrs. (K) shows relative mRNA levels of NOXA in 2008 and A2780 cells after treatment with 30 μM APR-246 for 24 hrs.

FIG. S1-4 shows that ARID1A-deficient cancer cells are selectively sensitive to PRIMA-1 and APR-246. Related to FIGS. 1-1 to 1-7. (L) shows detection of cleaved caspase-3/7-positive apoptotic cells in RMG-I, TOV21G and OVISE cells after treatment with 10 μM APR-246 for 24 hrs. (M) shows detection of cleaved caspase-3/7-positive apoptotic cells in 2008 and A2780 cells after treatment with 20 μM APR-246 for 16 hrs. All data are expressed as the mean±SD.

FIG. S2-1 shows that ARID1A-deficient cells are vulnerable to GSH inhibition. Related to FIGS. 2-1 to 2-5. (A) shows relative TrxR activity in ARID1A-WT and ARID1A-KO HCT116 cells after treatment with 50 μM APR-246 for 24 hrs. (B) and Figure S2-2 (C) show relative GSH levels (B) and relative ROS levels (C) in APR-246-treated ovarian (left; 16 hrs/5 μM), uterus (middle; 24 hrs/40 μM) and biliary tract (right; 48 hrs/40 μM) cancer cells.

FIG. S2-2 shows that ARID1A-deficient cells are vulnerable to GSH inhibition. Related to FIGS. 2-1 to 2-5. (D) to Figure S2-3 (F) show relative GSH levels (D), relative ROS levels (E), and cell viabilities (F) in ARID1A-deficient TOV21G (left) and OVISE (right) cancer cells after treatment with 20 μM APR-246 for 24 hrs and co-treatment with 5 mM NAC or 5 mM GSH-MEE.

FIG. S2-4 shows that ARID1A-deficient cells are vulnerable to GSH inhibition. Related to FIGS. 2-1 to 2-5. (G) shows colony formation of ARID1A-WT and ARID1A-KO HCT116 cells after treatment with the indicated concentrations of auranofin for 10 days. (H) shows cell viabilities of ovarian cancer cell lines after treatment with 0.4 μM APR-246 for 3 days. (I) shows detection of cleaved caspase-3/7-positive apoptotic cells in ARID1A-proficient 2008 and ARID1A-deficient TOV21G cells after treatment with 1 μM auranofin for 24 hrs. (J) to (K) show relative GSH levels (J) and relative ROS levels (K) in 2008 and TOV21G cells after treatment with 1 μM auranofin for 24 hrs. (L) shows ARID1A and p53 protein levels in ARID1A-WT and ARID1A-KO HCT116 cells expressing shNT (non-targeting shRNA) or shp53 (p53-targeting shRNA).

FIG. S2-5 shows that ARID1A-deficient cells are vulnerable to GSH inhibition. Related to FIGS. 2-1 to 2-5. (M) shows colony formation of ARID1A-WT and ARID1A-KO HCT116 cells expressing shNT or shp53 after treatment with the indicated concentrations of APR-246 for Figure S2-4 10 days. (N) shows detection of Annexin V-positive apoptotic cells in ARID1A-WT and ARID1A-KO HCT116 cells expressing shNT or shp53 after treatment with 22.5 μM APR-246 for 72 hr. (O) shows immunoblotting for ARID1A, p53, p21, and β-actin in whole-cell extracts of ARID1A-WT and ARID1A-KO HCT116 cells treated with DMSO, 50 μM PRIMA-1, 50 μM APR-246, and 10 μM Nutlin3a for 16 hrs.

FIG. S2-6 shows that ARID1A-deficient cells are vulnerable to GSH inhibition. Related to FIGS. 2-1 to 2-5. (P) shows relative mRNA levels of NOXA in ARID1A-KO HCT116 cells expressing shNT or shp53 after treatment with 50 μM APR-246 for 24 hrs. (Q) shows immunoblotting for JNK, phospho-JNK, NOXA, and β-actin in whole-cell extracts of ARID1A-KO HCT116 cells after treatment with 30 μM APR-246 for 24 hrs and co-treatment with 5 mM NAC or 10 μM of the JNK inhibitor SP600125 (JNKi). The red arrow head indicates the phosphorylated form of JNK. (R) shows relative mRNA levels of NOXA in ARID1A-KO HCT116 cells after treatment with 50 μM APR-246 for 24 hrs and co-treatment with 10 μM of the JNK inhibitor SP600125 (JNKi). (S) to Figure S2-7 (T) show relative GSH levels (S) and relative ROS levels (T) in ARID1A-KO HCT116 cells after treatment with 30 μM APR-246 for 24 and 48 hrs, respectively, and co-treatment with 10 μM of the JNK inhibitor SP600125 (JNKi).

FIG. S2-7 shows that ARID1A-deficient cells are vulnerable to GSH inhibition. Related to FIGS. 2-1 to 2-5. (U) shows relative mRNA levels of NOXA at 4 days after transfection into ARID1A-KO cells. (V) to (W) show relative GSH levels (V) and relative ROS levels (W) in ARID1A-KO HCT116 cells after treatment with 50 μM APR-246 for 24 and 48 hrs, respectively, 4 days after knockdown of the NOXA. Data in (A-K, M-N, P and R-W) are expressed as the mean±SD.

FIG. S3-1 shows that GCLC is a promising synthetic lethal target for ARID1A-deficient cancers. Related to FIGS. 3-1 to 3-7. (A) to (C) show heat maps of cell viabilities (A), relative GSH levels (B), and relative ROS levels (C) in ARID1A-proficient RMG-I cancer cells and ARID1A-deficient TOV21G cancer cells at 5, 3, and 3 days, respectively, after knockdown of TRXR and SOD family genes. (D) shows cell viabilities of ARID1A-WT and ARID1A-KO HCT116 cells to BSO, sulfasalazine, compound 968, and 6-aminonicotinamide after 5 days of treatment.

FIG. S3-2 shows that GCLC is a promising synthetic lethal target for ARID1A-deficient cancers. Related to FIGS. 3-1 to 3-7. (E) to (G) show relative mRNA levels of GCLC (E), GSS (F), and SLC7A11A (G) at 4 days after transfection in ovarian cancer cells. (H) to Figure S3-3 (J) show cell viabilities (H), relative GSH levels (I), and relative ROS levels (J) at 3 days after knockdown of GCLC, GSS, and SLC7A11 in ovarian cancer cells.

FIG. S3-3 shows that GCLC is a promising synthetic lethal target for ARID1A-deficient cancers. Related to FIGS. 3-1 to 3-7. (K) to (L) show relative mRNA levels of GCLC (K) and SLC7A11A (L) at 4 days after transfection in parental ARID1A-deficient TOV21G cells and TOV21G cells stably expressing ARID1A. (M) to Figure S3-4 (O) show relative GSH levels (M), relative ROS levels (N), and cell viabilities (O) at 3 days after knockdown of GCLC and SLC7A11 in parental ARID1A-deficient TOV21G cells and TOV21G cells stably expressing ARID1A.

FIG. S3-4 shows that GCLC is a promising synthetic lethal target for ARID1A-deficient cancers. Related to FIGS. 3-1 to 3-7. (P) to (S) show relative mRNA levels of GCLC (P) and cell viabilities (Q), relative GSH levels (R), and relative ROS levels (S) in ARID1A-WT and ARID1A-KO HCT116 cells at 7, 3, and 5 days after knockdown of GCLC.

FIG. S3-5 shows that GCLC is a promising synthetic lethal target for ARID1A-deficient cancers. Related to FIGS. 3-1 to 3-7. (T) shows relative mRNA levels of GCLC at 4 days after transfection in ARID1A-proficient 2008 and ARID1A-deficient TOV21G cells. (U) shows colony formation of 2008 and TOV21G cells at 14 days after knockdown of GCLC. (V) to (Y) show immunoblotting for GCLC and (β-actin in whole-cell extracts (V), relative GSH levels (W), relative ROS levels (X), and cell viabilities (Y) at 3 days after knockdown of GCLC in parental ARID1A-KO cells and ARID1A-KO HCT116 cells stably expressing GCLC. Data in (D-U and W-Y) are expressed as the mean±SD.

FIG. S4-1 shows that GCLC is a promising synthetic lethal target for ARID1A-deficient cancers. Related to FIGS. 3-1 to 3-7. (A) shows cell viabilities of cancer cell lines to BSO. (B) shows colony formation of ARID1A-proficient 2008, ARID1A-deficient TOV21G and OVISE cells at 14 days after treatment with 10 μM BSO. (C) to (D) show representative image (C) and survival rate (D) in colony formation assays of ARID1A-WT and ARID1A-KO cells after treatment with 300 μM BSO for 10 days.

FIG. S4-2 shows that GCLC is a promising synthetic lethal target for ARID1A-deficient cancers. Related to FIGS. 3-1 to 3-7. (E) shows relative fraction of Annexin V-positive apoptotic cells in ARID1A-WT and ARID1A-KO HCT116 cells after treatment with 500 μM APR-246 for 72 hr. (F) to (H) show relative GSH levels (F), relative ROS levels (G), and relative mRNA levels of NOXA (H) in RMG-I, TOV21G and OVISE cells after treatment with 20 μM BSO for 24 hrs.

FIG. S4-3 shows that GCLC is a promising synthetic lethal target for ARID1A-deficient cancers. Related to FIGS. 3-1 to 3-7. (I) to (J) show relative GSH levels (I) and relative ROS levels (J) in 2008 and A2780 cells after treatment with 300 μM BSO for 24 and 48 hrs, respectively. (K) to (M) show relative GSH levels (K), relative ROS levels (L), and cell viabilities (M) in parental TOV21G cells and TOV21G cells stably expressing ARID1A after treatment with 100 μM BSO for 24, 48, and 48 hrs, respectively.

FIG. S4-4 shows that GCLC is a promising synthetic lethal target for ARID1A-deficient cancers. Related to FIGS. 3-1 to 3-7. (N) to Figure S4-5 (P) show relative GSH levels (N), relative ROS levels (O), and cell viabilities (P) in ARID1A-deficient TOV21G (upper) and OVISE (lower) cancer cells after treatment with 10 and 20 μM BSO, respectively, for 24 hrs in the presence or absence of 5 mM NAC or 5 mM GSH-MEE.

FIG. S4-5 shows that GCLC is a promising synthetic lethal target for ARID1A-deficient cancers. Related to FIGS. 3-1 to 3-7. (Q) to Figure S4-6 (S) show relative GSH levels (Q), relative ROS levels (R), and cell viabilities (S) of parental TOV21G cells and TOV21G cells stably expressing GCLC after treatment with 20 μM APR-246 for 24, 24, and 48 hrs, respectively. Data in (A-B and D-S) are expressed as the mean±SD.

FIG. S5-1 shows that vulnerability of ARID1A-deficient cancers to GSH inhibition is caused by decreased GSH synthesis due to impaired SLC7A11 expression. Related to FIGS. 4-1 to 4-7. (A) to (C) show relative mRNA expression (left) and protein expression (right) of SLC7A11 in ovarian (A), uterus (B), and biliary tract (C) cancer cells.

FIG. S5-2 shows that vulnerability of ARID1A-deficient cancers to GSH inhibition is caused by decreased GSH synthesis due to impaired SLC7A11 expression. Related to FIGS. 4-1 to 4-7. (D) shows relative mRNA expression (left) and protein expression (right) of NRF2 in ARID1A-WT cells, parental HCT116 ARID1A-KO cells, and ARID1A-KO cells stably expressing NRF2. (E) shows relative mRNA expression (left) and protein expression (right) of SLC7A11 in ARID1A-WT cells, parental HCT116 ARID1A-KO cells, and ARID1A-KO cells stably expressing NRF2. (F) shows cell viabilities in parental TOV21G cells and TOV21G cells stably expressing NRF2 after treatment with 3.7 μM APR-246 for 6 days.

FIG. S5-3 shows that vulnerability of ARID1A-deficient cancers to GSH inhibition is caused by decreased GSH synthesis due to impaired SLC7A11 expression. Related to FIGS. 4-1 to 4-7. (G) shows representative image of (left), and survival rates in (right), colony formation assays of ARID1A-WT and ARID1A-KO HCT116 cells after treatment with 2 μM APR-246 for 10 days. (H) to (I) show relative GSH levels (H) and relative ROS levels (I) in parental TOV21G cells and TOV21G cells stably expressing NRF2 following treatment with 20 μM APR-246 for 24 and 48 hrs, respectively. (J) shows basal cysteine levels in ARID1A-WT and ARID1A-KO HCT116 cells (left) and ARID1A-proficient 2008 and ARID1A-deficient A2780 cells (right). Data are expressed as the mean±SEM (n=3). *p<0.05, ***p<0.001; two-tailed t-test.

FIG. S5-4 shows that vulnerability of ARID1A-deficient cancers to GSH inhibition is caused by decreased GSH synthesis due to impaired SLC7A11 expression. Related to FIGS. 4-1 to 4-7. (K) shows basal GSH levels in ARID1A-WT and ARID1A-KO HCT116 cells. (L) shows basal GSH levels in ovarian (left), uterus (middle), and biliary tract (right) cancer cells.

FIG. S5-5 shows that vulnerability of ARID1A-deficient cancers to GSH inhibition is caused by decreased GSH synthesis due to impaired SLC7A11 expression. Related to FIGS. 4-1 to 4-7. (M) shows basal ROS levels in ARID1A-proficient RMG-I cancer cells and ARID1A-deficient TOV21G cancer cells. (N) shows basal ROS levels in ARID1A-WT and ARID1A-KO HCT116 cells. (O) shows basal ROS levels in ovarian (left), uterus (middle), and biliary tract (right) cancer cells.

FIG. S5-6 shows that vulnerability of ARID1A-deficient cancers to GSH inhibition is caused by decreased GSH synthesis due to impaired SLC7A11 expression. Related to FIGS. 4-1 to 4-7. (P) shows basal ROS levels in parental TOV21G cells and TOV21G cells stably expressing ARID1A. (Q) shows basal ROS levels in parental TOV21G cells and TOV21G cells stably expressing SLC7A11. (R) to (S) show basal GSH levels (R) and basal ROS levels (S) in parental TOV21G cells and TOV21G cells stably expressing GCLC.

FIG. S5-7 shows that vulnerability of ARID1A-deficient cancers to GSH inhibition is caused by decreased GSH synthesis due to impaired SLC7A11 expression. Related to FIGS. 4-1 to 4-7. (T) to (V) show relative GSH levels (T), relative ROS levels (U), and cell viabilities (V) in parental TOV21G cells and TOV21G cells stably expressing SLC7A11 following treatment with 50 μM BSO for 24, 24, and 48 hrs, respectively. Data in (A-I and K-V) are expressed as the mean±SD.

FIG. S6-1 shows clinical relevance of GSH-targeting therapy for ARID1A-deficient cancers. Related to FIGS. 5-1 to 5-5 and FIGS. 6-1 to 6-4. (A) shows ARID1A gene status and percentage of cells staining positively for ARID1A and SLC7A11 of eleven ovarian cancer specimens subjected to immunohistochemical analysis are shown. (B) shows immunohistochemical staining for ARID1A and SLC7A11 in surgically resected ovarian tumors with mutant ARID1A retaining ARID1A protein expression. Scale bar, 50 μm.

FIG. S6-2 shows clinical relevance of GSH-targeting therapy for ARID1A-deficient cancers. Related to FIGS. 5-1 to 5-5 and FIGS. 6-1 to 6-4. (C) shows cell viabilities of ovarian PDCs after treatment with 100 μM BSO for 6 days. (D) shows basal ROS levels in ovarian PDCs. (E) shows detection of cleaved caspase-3/7-positive apoptotic cells in ovarian PDCs after treatment with 50 μM APR-246 for 24 hrs.

FIG. S6-3 shows clinical relevance of GSH-targeting therapy for ARID1A-deficient cancers. Related to FIGS. 5-1 to 5-5 and FIGS. 6-1 to 6-4. (F) shows body weight of mice treated with 50 mg/kg APR-246 (n=6). (G) shows tumor volume of xenografts derived from ARID1A-deficient OVISE OCCC cancer cells in mice treated with 25 mg/kg PRIMA-1 (n=5). (H) shows body weight of mice treated with 25 mg/kg PRIMA-1. (n=5). (I) shows tumor volume of xenografts derived from ARID1A-deficient TOV21G OCCC cancer cells in mice treated with 750 mg/kg BSO once a day for 14 days (n=6). (J) to Figure S6-4 (K) show relative tumor weight of xenografts derived from ARID1A-deficient TOV21G OCCC cancer cells (J) and body weight of mice (K) treated with 750 mg/kg BSO once a day for 14 days (n=6).

FIG. S6-4 shows clinical relevance of GSH-targeting therapy for ARID1A-deficient cancers. Related to FIGS. 5-1 to 5-5 and FIGS. 6-1 to 6-4. (L) to (M) show immunoblotting for GCLC and β-actin (L), and the GCLC protein levels (M) of TOV21G-shNT (left) and TOV21G-shGCLC (right) xenografts in mice treated with Dox. After tumor formation, mice were fed a diet with or without Dox (n=6). (N) to Figure S6-5 (O) show tumor volume of TOV21G-shNT (N) and TOV21G-shGCLC (O) xenografts in mice treated with Dox. After inoculation of these cells, mice were fed a diet with or without Dox (n=6).

FIG. S6-5 shows clinical relevance of GSH-targeting therapy for ARID1A-deficient cancers. Related to FIGS. 5-1 to 5-5 and FIGS. 6-1 to 6-4. (P) to (Q) show relative tumor weight of TOV21G-shNT (P) and TOV21G-shGCLC (Q) xenografts in mice treated without or with Dox (n=6). (R) shows relative GSH levels in TOV21G-shNT and TOV21G-shGCLC xenografts in mice treated with Dox (n=6).

FIG. S6-6 shows clinical relevance of GSH-targeting therapy for ARID1A-deficient cancers. Related to FIGS. 5-1 to 5-5 and FIGS. 6-1 to 6-4. (S) shows immunohistochemical staining for the oxidative stress marker 8-0HdG; the apoptosis markers NOXA, cleaved caspase-3 and cleaved PARP; and the cell proliferation marker Ki67 in TOV21G-shGCLC xenografts in mice treated with Dox. Scale bar, 50 μm. Data in (C-E), are expressed as the mean±SD. Data in (F-K and M-R), are expressed as the mean±SEM. *p<0.05, **p<0.01, and ***p<0.001; two-tailed t-test.

FIG. S7 shows that ARID1A-deficient gastric cancer cells are vulnerable to GSH inhibition due to low basal levels of GSH. (A) shows relative expression of SLC7A11 mRNA in PDCs: ARID1A-WT NSC-64C cells and ARID1A-deficient NSC-7C cells. Data are expressed as the mean±SD. (B) shows basal GSH levels in PDCs: ARID1A-WT NSC-64C cells and ARID1A-deficient NSC-7C cells. Data are expressed as the mean±SD. (C) shows relative levels of GSH/GSSG, which indicates the ratio of reduced GSH to the oxidized form GSH disulfide (GSSG), in ARID1A-WT NSC-64C cells and ARID1A-deficient NSC-7C cells treated with 50 μM APR-246 for 24 hrs. Data are expressed as the mean±SD. (D) shows relative ROS levels in ARID1A-WT NSC-64C cells and ARID1A-deficient NSC-7C cells treated with 50 μM APR-246 for 48 h. Data are expressed as the mean±SD.

FIG. S8 shows that ARID1A-deficient diffuse-type gastric cancer PDCs are sensitive to erastin. (A) shows IC50 values for erastin in DGC PDCs including four ARID1A-WT PDCs and ARID1A-deficient PDCs after treatment for 6 days. Data are expressed as means±SEM. (n=4) *p<0.05; two-tailed Student's t-test. (B) shows relative levels of GSH/GSSG, which indicates the ratio of reduced GSH to the oxidized form, GSH disulfide (GSSG), in ARID1A-WT NSC-48CA cells and ARID1A-deficient NSC-67C cells treated with 1.25 μM erastin for 24 hrs. Data are expressed as means±SD.

### DETAILED DESCRIPTION

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise.

The present disclosure also contemplates other embodiments “comprising,” “consisting of” and “consisting essentially of,” the embodiments or elements presented herein, whether explicitly set forth or not.

The term “about” used in connection with a quantity is inclusive of the stated value and has the meaning dictated by the context (for example, it includes at least the degree of error associated with the measurement of the particular quantity). The term “about” should also be considered as disclosing the range defined by the absolute values of the two endpoints. The term “about” may refer to plus or minus 10% of the indicated number.

### DISCUSSION

The present invention relates to a method for treating SWI/SNF complex-deficient cancer, comprising administering an effective amount of a glutathione (GSH) metabolic pathway inhibitor to a mammal in need thereof.

A “glutathione (GSH) metabolic pathway inhibitor” includes a substance for inhibiting GSH itself, or GSH synthesis enzymes. For example, a glutathione (GSH) metabolic pathway inhibitor includes a glutamate-cysteine ligase synthetase (GCL) inhibitor, a glutamate-cysteine ligase catalytic subunit (GCLC) inhibitor, a glutamate-cysteine ligase modifier (GCLM) inhibitor, a GSH synthetase (GSS) inhibitor, an SLC7A11 inhibitor, or a combination thereof.

Among them, a GCLC inhibitor, which inhibits a glutamate-cysteine ligase catalytic subunit (GCLC), i.e., a rate-limiting enzyme in a GSH biosyntheis pathway, is particularly preferable. The representative example of a GCLC inhibitor is buthionine sulfoximine (BSO) (Cayman Chemical Co.) (CAS Number: 83730-53-4). Among them, a GCL inhibitor is also preferable.

SLC7A11 inhibitor is, for example, sulfasalazine (Pfizer).

A glutathione (GSH) metabolic pathway inhibitor may be also antibody, small interfering RNA (siRNA), short hairpin RNA (shRNA), microRNA (miRNA), antisense oligonucleotide (ASO), low-molecular compound, a combination thereof, and the like.

As used herein, a “low-molecular compound” means a low molecular weight organic compound that may serve as an enzyme substrate or regulator of biological processes. In general, a low-molecular compound is less than about 5 kilodaltons (kD) in size. In some embodiments, in accordance with the present invention, a low-molecular compound is not proteins, polypeptides, oligopeptides, peptides, polynucleotides, oligonucleotides, polysaccharides, glycoproteins, proteoglycans, and the like. In some embodiments, a small molecule may be a therapeutic, an adjuvant, or a drug.

A glutathione (GSH) metabolic pathway inhibitor may be a “GSH reduction compound,” which inhibits GSH itself, and as a result, reduces the GSH level in cells. A GSH reduction compound is, for example, APR-017 (also known as Anti-P2Y13 Receptor Antibody or PRIMA-1; Alomone Labs.), or APR-246 (also known as PRIMA-1MET; Aprea Therapeutic) (CAS Number: 5291-32-7).

As used herein, component(s) of “SWI/SNF complex(es)” include(s), for example, ACTB, ACTL6A (BAF53A), ACTL6B (BAF53B), ARID1A (BAF250A), ARID1B (BAF250B), ARID2 (BAF200), BCL11A, BCL11B, BCL7A, BCL7B, BCL7C, BICRA (GLTSCR1), BRD7, BRD9, DPF1 (BAF45B), DPF2 (BAF45D), DPF3 (BAF45C), PBRM1 (BAF180), PHF10 (BAF45A), SMARCA2 (BRM), SMARCA4 (BRG1, BAF190), SMARCB1 (BAF47, hSNF5, INI1), SMARCC1 (BAF155), SMARCC2 (BAF170), SMARCD1 (BAF60A), SMARCD2 (BAF60B), SMARCD3 (BAF60C), SMARCE1 (BAF57), SS18, or a combination thereof. Among them, ARID1A, SMARCA2, SMARCA4, SMARCB1, PBRM1 or a combination thereof is preferable. In particular, ARID1A is more preferable.

As used herein, in some cases, “SWI/SNF complex(es)” mean(s) each component(s) or a combination thereof.

As used herein, “SWI/SNF complex(es) deficiency” means SWI/SNF complex(es) gene(s) deficiency or SWI/SNF complex(es) protein(s) deficiency caused by SWI/SNF complex(es) gene mutation. Herein, “SWI/SNF complex(es) deficiency” may be caused by which each of genes belonging to SWI/SNF complex(es) or a combination of genes belonging to SWI/SNF complex(es) are mutated. “SWI/SNF complex(es)-deficient cancer” means cancer having SWI/SNF complex(es) deficiency.

As used herein, “ARID1A deficiency” means ARID1A gene deficiency or ARID1A protein deficiency caused by ARID1A gene mutation. “ARID1A-deficient cancer” means cancer having ARID1A deficiency. Examples of ARID1A-deficient cancer include ovarian cancer, uterine cancer, gastric cancer, bladder cancer, bile duct cancer, liver cancer, esophageal cancer, lung cancer, colon cancer, pancreatic cancer, breast cancer, neuroblastoma, glioma, skin cancer, B-cell lymphoma, renal cancer, and the like.

As used herein, “SMARCA2 (BRM) deficiency” means SMARCA2 (BRM) gene deficiency or SMARCA2 (BRM) protein deficiency caused by SMARCA2 (BRM) gene mutation. “SMARCA2 (BRM)-deficient cancer” means cancer having SMARCA2 (BRM) deficiency. Examples of SMARCA2 (BRM)-deficient cancer include adenoid cystic carcinoma, bladder cancer, breast cancer, cervical cancer, colon cancer, esophageal cancer, gastric cancer, glioma, head and neck cancer, lung cancer, medul-loblastoma, melanoma, pancreatic cancer, ovarian cancer, prostate cancer, SMARCA4-deficient thoracic sarcoma, uterine cancer, sarcoma, and the like.

As used herein, “SMARCA4 (BRG1) deficiency” means SMARCA4 (BRG1) gene deficiency or SMARCA4 (BRG1) protein deficiency caused by SMARCA4 (BRG1) gene mutation. “SMARCA4 (BRG1)-deficient cancer” means cancer having SMARCA4 (BRG1) deficiency. Examples of SMARCA4 (BRG1)-deficient cancer include bladder cancer, breast cancer, cervical cancer, colon cancer, B-cell lymphoma, esophageal cancer, gastric cancer, glioma, head and neck cancer, liver cancer, lung cancer, melanoma, pancreatic cancer, prostate cancer, renal cancer, rhabdoid tumor, ovarian cancer, SMARCA4-deficient thoracic sarcoma, uterine cancer and the like.

As used herein, “SMARCB1 deficiency” means SMARCB1 gene deficiency or SMARCB1 protein deficiency caused by SMARCB1 gene mutation. “SMARCB1-deficient cancer” means cancer having SMARCB1 deficiency. Examples of SMARCB1-deficient cancer include epithelioid sarcoma, familial schwannomatosis, gastric cancer, renal cancer, rhabdoid tumor, sinonasal carcinoma, synovial sarcoma, undifferentiated chordomas, uterine cancer and the like.

As used herein, “PBRM1 deficiency” means PBRM1 gene deficiency or PBRM1 protein deficiency caused by PBRM1 gene mutation. “PBRM1-deficient cancer” means cancer having PBRM1 deficiency. Examples of PBRM1-deficient cancer include bladder cancer, bile duct cancer, colon cancer, B-cell lymphoma, esophageal cancer, gastric cancer, head and neck cancer, lung cancer, melanoma, mesothelioma, pancreatic cancer, renal cancer, uterine cancer and the like.

As used herein, ovarian cancer, includes, for example, ovarian clear cell carcinoma or ovarian endometrioid carcinoma.

There are a large number of patients suffering from ovarian clear cell carcinoma in Japan, and ARID1A-deficient ovarian cancer is found at a high rate, i.e., in one of two of ovarian cancer patients.

As used herein, uterine cancer is, for example, uterine corpus endometrial carcinoma. As used herein, the term “treatment” (also “treat” or “treating”) refers to any administration of a glutathione (GSH) metabolic pathway inhibitor that partially or completely alleviates, ameliorates, relives, inhibits, delays onset of, reduces severity of, and/or reduces frequency, incidence or severity of one or more symptoms, features, and/or causes of SWI/SNF complex(es)-deficient cancer. Such treatment may be of a subject who does not exhibit signs of the relevant disease, disorder and/or condition (e.g., may be prophylactic) and/or of a subject who exhibits only early signs of the disease, disorder, and/or condition. Alternatively or additionally, such treatment may be of a subject who exhibits one or more established signs of the relevant disease, disorder and/or condition (e.g., may be therapeutic).

As used herein, the term “effective amount” refers to an amount effective, at dosages and for periods of time necessary, to achieve the desired therapeutic result. An effective amount of the composition may be determined by a person skilled in the art and may vary according to factors such as the disease state, age, sex, and weight of the individual, and the ability of the composition to elicit a desired response in the individual. An effective amount is also one in which any toxic or detrimental effects of an inhibitor are outweighed by the therapeutically beneficial effects.

The effective amount of a glutathione (GSH) metabolic pathway inhibitor may be adjusted appropriately to achieve desired drug levels, local or systemic, depending upon the mode of administration. Specifically, the effective amount is preferably about 0.5 ng to about 2000 mg per day at a time, or about 1 ng to about 1000 mg per day at a time, and it may be administered to patients once to several times per day.

As used herein, the term “mammal” refers to humans or non-human animals. In some embodiments, the non-human animal includes, for example, a rodent, a mouse, a rat, a rabbit, a monkey, a dog, a cat, a sheep, cattle, a primate, a pig, and the like.

As used herein, the term “administer” (also, administration, administering) refers to administering the present inhibitor or a composition comprising the same to the subject (mammal). The route by which the disclosed inhibitor or composition is administered and the form thereof will dictate the type of carrier to be used. The inhibitor or composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

The inhibitor or the pharmaceutical compositions according to the present invention can be prepared by processes which are known per se and familiar to the person skilled in the art. As pharmaceutical compositions, the active agents are either employed as such, or preferably in combination with suitable pharmaceutical auxiliaries and/or excipients, e.g. in the form of tablets, coated tablets, capsules, caplets, suppositories, patches, emulsions, suspensions, gels or solutions. A person skilled in the art is familiar with auxiliaries, vehicles, excipients, diluents, carriers or adjuvants which are suitable for the desired pharmaceutical formulations, preparations or compositions on account of his/her expert knowledge. In addition to solvents, gel formers, ointment bases and other excipients, for example antioxidants, dispersants, emulsifiers, preservatives, solubilizers, colorants, complexing agents or permeation promoters, can be used.

The present invention also relates to a method for detecting and/or selecting a susceptible patient to a GSH metabolic pathway inhibitor, comprising detecting the presence of SWI/SNF complex(es) deficiency in a patient having cancer.

As used herein, “susceptible patient to a GSH metabolic pathway inhibitor” means patient having cancers which are sensitive to a GSH metabolic pathway inhibitor.

As used herein, the term “sample” includes a tissue sample (brain, hair, buccal swabs, blood, saliva, semen, muscle, any internal organs, or cancer, precancerous, tumor cells), cell sample, a fluid sample (urine, blood, ascites, pleural fluid, spinal fluid), and the like.

Specifically, in some embodiment, a method for detecting and/or selecting a susceptible patient to a GSH metabolic pathway inhibitor, according to the present invention, comprises (i) providing a sample derived from a patient having cancer, (ii) measuring the level of SWI/SNF complex(es) in the sample, (iii) comparing the measued level with the predetermined value, and (iv) it is determined that if the measued level is below the predetermined value, SWI/SNF complex(es) is deficient and that the patient is likely to be sensitive to a GSH metabolic pathway inhibitor, while if the measued level is above the predetermined value, it is determined that SWI/SNF complex(es) is not deficient and that the patient is unlikely to be sensitive to a GSH metabolic pathway inhibitor.

In some embodiment, a method for detecting and/or selecting a susceptible patient to a GSH metabolic pathway inhibitor, according to the present invention, comprises (i) providing a sample derived from a patient having cancer, (ii) measuring the level of SWI/SNF complex(es) gene expression in the sample, (iii) comparing the measured level with the predetermined value, and (iv) it is determined that if the measured level is below the predetermined value, SWI/SNF complex(es) gene is deficient and that the patient is likely to be sensitive to a GSH metabolic pathway inhibitor, while if the measued level is above the predetermined value, it is determined that SWI/SNF complex(es) gene is not deficient and that the patient is unlikely to be sensitive to a GSH metabolic pathway inhibitor.

The predetermined value may be the level of SWI/SNF complex(es) gene or protein in normal tissue sample from unaffected individuals.

The presence of SWI/SNF complex(es) deficiency in the sample is determined by, for example, Cancer Gene Panel Test (OncoGuide™ NCC Oncopanel System).

The present invention also relates to a kit. The kit may include information, instructions, or both that use of the kit will provide treatment for medical conditions in mammals (particularly humans). The information and instructions may be in the form of words, pictures, or both, and the like. Additionally or alternatively, the kit may include the inhibitor, the composition, or both; and information, instructions, or both, regarding methods of application of the inhibitor or the composition, preferably with the benefit of treating or preventing medical conditions in mammals (e.g., humans).

In this study, selective sensitivity to GSH metabolic pathway inhibitors such as APR-246 according to SWI/SNF complex(es) deficiency, was observed in several cancer cell lines. For example, with regard to ARID1A deficiency, HCT116 colon cancer cells with or without artificial gene knockout; widely used ovarian, uterine, gastric and biliary tract cancer cell lines; and newly established ovarian patient derived cells (PDCs). Based on results herein, it was demonstrated that ARID1A-mutated tumors without ARID1A protein expression, but not those retaining ARID1A expression, respond well to GSH inhibitor- or GSH metabolic pathway inhibitor-targeted therapy. Thus, immunohistochemical assessment of ARID1A protein will be a useful diagnostic tool for detecting and/or selecting patients suitable for GSH inhibitor- or GSH metabolic pathway inhibitor-targeted therapy.

### EXAMPLES

The following examples are set forth so as to provide a person skilled in the art with a complete disclosure and description of how the methods claimed wherein are made and evaluated, and are intended to purely exemplary of the invention and are not intended to limit the scope of what the inventors regard as the invention.

### Example 1: ARID1A-Deficient Cancer Cells are Selectively Sensitive to PRIMA-1 and APR-246

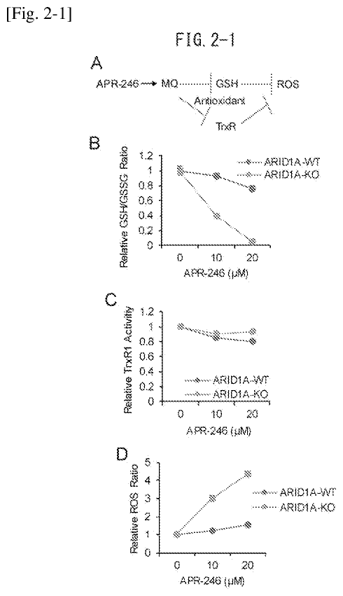

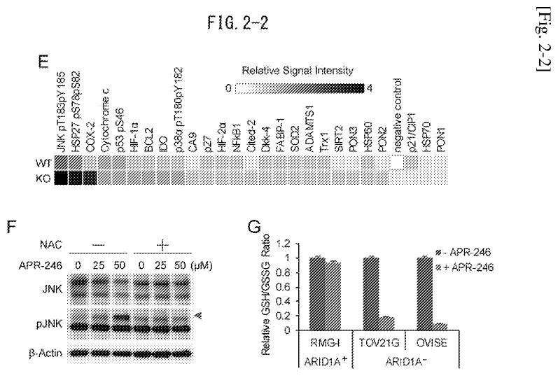

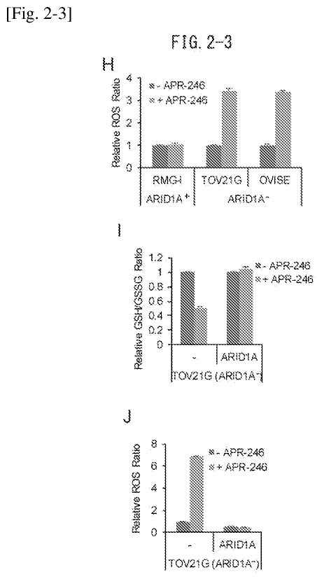

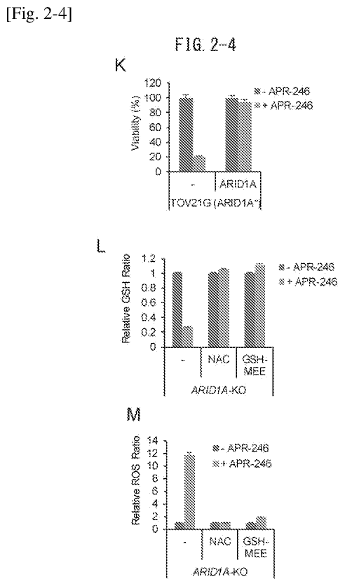

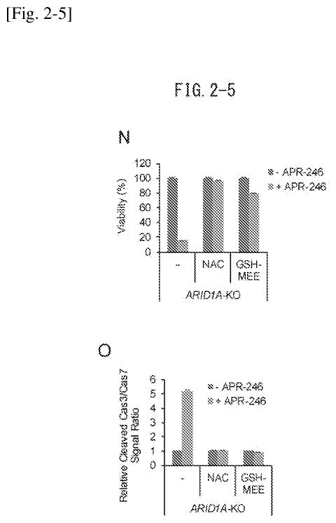

To investigate cellular vulnerabilities caused by ARID1A deficiency, we examined the differential sensitivities of parental ARID1A-wild-type (ARID1A-WT) and ARID1A-knockout (ARID1A-KO) HCT116 colon cancer cells to 334 inhibitors whose targets have been elucidated. A drug sensitivity screen identified PRIMA-1 (APR-017) (FIG. 1-1 A-C, Table S1), which binds covalently to thiols in multiple polypeptides (See, Bykov et al., Nat Med 8, 282-288, 2016), as a unique hit that reproducibly suppressed the growth of ARID1A-KO cells but not ARID1A-WT cells (FIG. 1-2 D). The selective sensitivity of ARID1A-KO cells to PRIMA-1 was validated by measuring cell survival in colony formation assays (FIG. 1-2 E, Figure S1-1 A). APR-246 (PRIMA-1Met), a structural analog of PRIMA-1, has been tested in clinical trials of hematological and prostate cancers (See, Lehmann et al., J Clin Oncol 30, 3633-3639, 2012). APR-246 markedly decreased the survival of ARID1A-KO cells, but not ARID1A-WT cells (FIG. 1-2 F-G, Figure S1-1 B). Next, we tested the sensitivity of various ARID1A-deficient cancer cells to APR-246 and PRIMA-1. This analysis used a panel of four ARID1A-proficient and six ARID1A-deficient cell lines (FIG. 1-3 H). ARID1A-deficient cancer cells were more sensitive to APR-246 than ARID1A-proficient cancer cells (FIG. 1-4 I, Figures S1-1 C to S1-2 E). The selective sensitivity of ARID1A-deficient cancer cells to APR-246 was validated by measuring cell survival in colony formation assays (FIG. 1-4 J, Figure S1-2 F). Similar results were obtained in PRIMA-1 treatment (Figures S1-2 G to S1-3 H). The preferential lethality of APR-246 and PRIMA-1 in TOV21G ARID1A-deficient cancer cells was rescued by stable expression of the ARID1A cDNA (FIG. 1-4 K, Figure S1-3 I), confirming that ARID1A deficiency was responsible for sensitivity to these drugs, irrespective of the cellular context.

To explore the sensitivity of ARID1A-KO cells to APR-246, genome-wide expression profiling was conducted. A pathway analysis of 6,429 genes whose expression levels in ARID1A-KO cells, but not in ARID1A-WT cells, increased or decreased by more than 2-fold upon APR-246 treatment was performed to identify the pathways responsible for the sensitivity of ARID1A-KO cells to ARP-246 (FIG. 1-5 L). Pathways related to apoptosis were significantly (p<0.001) enriched among these genes (FIG. 1-5 L). Notably, several key pro-apoptotic genes, including NOXA, were markedly upregulated in ARID1A-KO cells, but not in ARID1A-WT cells (FIG. 1-6 M). Quantitative RT-PCR analysis verified that mRNA expression of NOXA increased in APR-246-treated ARID1A-KO cells and ARID1A-deficient cancer cells (FIG. 1-7 N, Figures S1-3 J to S1-3 K). Concordantly, protein expression of NOXA increased specifically in APR-246-treated ARID1A-KO cells (FIG. 1-7 O). Moreover, APR-246 treatment induced apoptosis, as assessed by caspase activation and Annexin V appearance, in ARID1A-KO cells and ARID1A-deficient cancer cells (FIGS. 1-7 P to 1-7 R, Figures S1-4 L to S1-4 M).

### Example 2: ARID1A-Deficient Cancer Cells are Vulnerable to GSH Inhibition

APR-246 is converted to the Michael acceptor methylene quinuclidinone (MQ), which inhibits activity of the antioxidant metabolite GSH and the antioxidant regulator thioredoxin reductase (TrxR) by reacting with their thiols (See, Peng et al., Cell Death Dis 4, e881, 2013; Tessoulin et al., Blood 124, 1626-1636, 2014). Covalent binding of MQ decreased the level of GSH and inhibited TrxR activity, thereby shifting the intracellular balance between ROS generation and antioxidation toward an increase in ROS levels (FIG. 2-1 A). Therefore, we next examined whether the lethal effects of APR-246 on ARID1A-deficient cells were due to inhibition of GSH and TrxR. APR-246 treatment dose-dependently decreased the GSH level [presented as the ratio of reduced GSH to its oxidized form, GSH disulfide (GSSG)] in ARID1A-KO cells, but not in ARID1A-WT cells (FIG. 2-1 B). On the other hand, the same concentrations of APR-246 did not affect TrxR activity (including TrxR1, TrxR2 and TrxR3) in ARID1A-WT or ARID1A-KO cells, while a higher concentration of APR-246 suppressed TrxR activity (FIG. 2-1 C, Figure S2-1 A). Notably, the intracellular ROS level, as indicated by intracellular H2O2, was increased more in ARID1A-KO cells than in ARID1A-WT cells (FIG. 2-1 D). The increase in ROS level upon APR-246 treatment is consistent with the previous finding that APR-246 binds to and suppresses the functions of antioxidants such as GSH (See, Bykov et al., Front Oncol 6, 21, 2016). Notably, the ROS level was increased much more in ARID1A-KO cells than in ARID1A-WT cells, indicating that the level of oxidative stress induced by APR-246 treatment was higher in ARID1A-KO cells than in ARID1A-WT cells—that is, the balance between ROS generation and antioxidation shifted toward a more oxidized state due to the decreased GSH level in ARID1A-KO cells. Indeed, activation of the p38 MAPK and EGFR signaling pathways, which are known to be activated by oxidative stress (See, Matsuzawa and Ichijo, Biochim Biophys Acta 1780, 1325-1336, 2008; Weng et al., J Exp Clin Cancer Res 37, 61, 2018), was more evident in ARID1A-KO cells treated with ARP-246 (FIG. 1-5 L). In addition, an antibody array analysis demonstrated that levels of cyclooxygenase-2 (COX-2), phosphorylated JNK, and phosphorylated HSP27, all of which are induced by oxidative stress, were higher in ARID1A-KO cells than in ARID1A-WT cells (FIG. 2-2 E). Phosphorylation of JNK in response to APR-246 was suppressed by co-treatment with the ROS scavenger N-acetyl cysteine (NAC) (FIG. 2-2 F). These results indicate that APR-246 induced a state of high oxidative stress in ARID1A-KO cells.

The decreased GSH level and increased ROS level after APR-246 treatment were only evident in ARID1A-deficient human ovarian and other cancer cells (FIGS. 2-2 G to 2-3 H, Figures S2-1 B to S2-2 C). Stable introduction of the ARID1A cDNA into ARID1A-deficient cancer cells rescued the APR-246-induced decrease in GSH, increase in ROS, and inhibition of cell growth (FIGS. 2-4 I to 2-4 K), indicating that sensitivity to APR-246 was dependent on ARID1A function. In addition, the GSH decrease and ROS increase in APR-246-treated ARID1A-KO cells were completely suppressed by co-treatment with the ROS scavenger NAC or the GSH compensator glutathione monoethyl ester (GSH-MEE) (FIGS. 2-4 L to 2-4 M). Consistently, the decrease in cell viability and induction of apoptosis in APR-246-treated ARID1A-KO cells were completely abolished by co-treatment with NAC or GSH-MEE (FIGS. 2-5 N to 2-5 O). Similar results were obtained in two different ARID1A-deficient cancer cell lines (Figures S2-2 D to S2-3 F). These results indicate that inhibition of GSH in ARID1A-deficient cancer cells with APR-246 leads to higher ROS levels by disrupting the balance between ROS generation and antioxidation by GSH, resulting in apoptotic cell death.

MQ derived from APR-246 and PRIMA-1 also binds covalently to cysteine residues in TrxR (See, Bykov et al., Front Oncol 6, 21, 2016). In fact, ARID1A-KO cells and ARID1A-deficient cells were more sensitive to the TrxR inhibitor auranofin than ARID1A-proficient cells (Figures S2-4 G to 2-4 I). The decrease in the GSH level and the increase in the ROS level by auranofin were more evident in ARID1A-deficient cancer cells, as in the case of APR-246 (Figures S2-4 J to S2-4 K). These results indicate that the sensitivity of ARID1A-deficient cancer cells to APR-246 is due mainly to GSH inhibition, as shown in FIGS. 2-1 B to 2-1 C, but might be also partially due to inhibition of TrxR. MQ also binds to and activates mutant p53, leading to induction of apoptosis (See, Bykov et al., Nat Med 8, 282-288, 2002; Lambert et al., Cancer Cell 15, 376-388, 2009). HCT116 cells express wild-type TP53; therefore, it is unlikely that p53 is the target underlying the sensitivity of ARID1A-deficient cells to APR-246. Indeed, depletion of endogenous wild-type p53 did not significantly affect APR-246-induced apoptosis in ARID1A-KO cells (Figures S2-4 L to S2-5 N). In addition, treatment with Nutlin3a, an MDM2 inhibitor that stabilizes wild-type p53, increased the p53 level and upregulated the p53 target p21, whereas treatment with PRIMA-1 and APR-246 at concentrations that induced apoptosis did not markedly affect expression of p53 or p21 (Figure S2-5 O). These findings indicate that the sensitivity of ARID1A-deficient cancer cells to APR-246 is not dependent on p53. NOXA is a transcriptional target of p53; however, NOXA expression induced by APR-246 was not suppressed by p53 depletion in ARID1A-KO cells (Figure S2-6 P). Induction of NOXA is also caused by JNK signaling pathway activation (See, Wang et al., Mol Carcinog 47, 436-445, 2008). Indeed, NOXA expression induced by APR-246 was also suppressed by co-treatment with a JNK inhibitor (Figures S2-6 Q to S2-6 R) or with the ROS scavenger NAC (FIG. 2-2F). Thus, JNK signaling activated by oxidative stress caused NOXA upregulation in ARID1A-KO cells. However, the decrease in the GSH level and the increase in the ROS level in APR-246-treated ARID1A-KO cells were not suppressed by co-treatment with the JNK inhibitor or by NOXA depletion (Figures S2-6 S to S2-7 W). Therefore, activation of the JNK signaling pathway and the resulting increase in NOXA expression seem to be secondary effects of oxidative stress and not the mediators causing lethality.

### Example 3: GCLC is a Promising Synthetic Lethal Target in ARID1A-Deficient Cancers

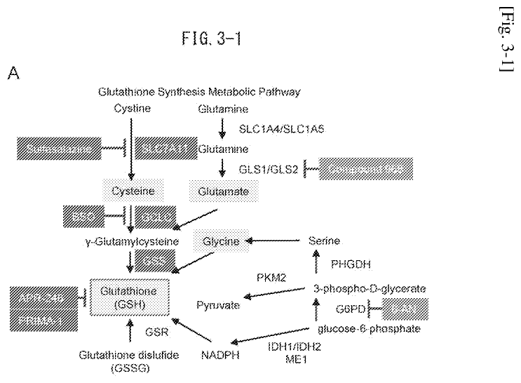

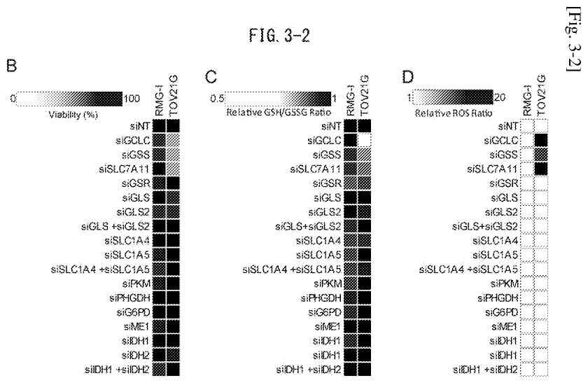

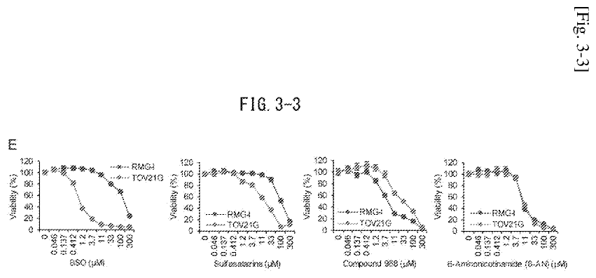

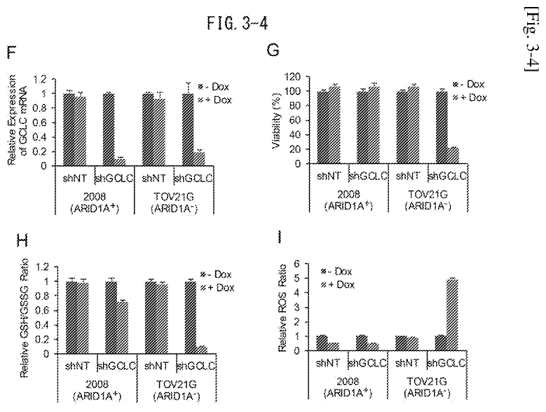

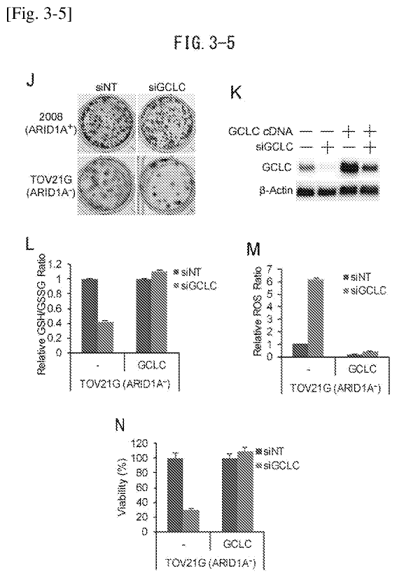

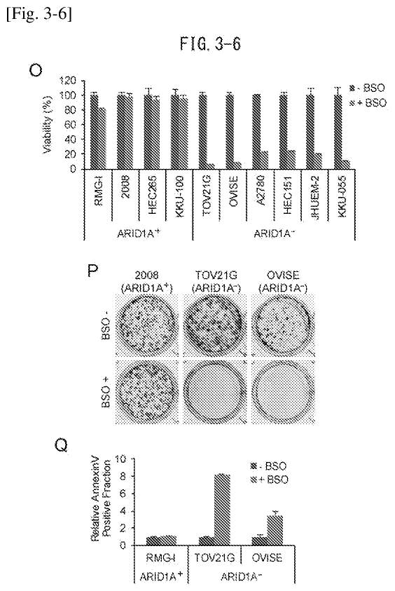

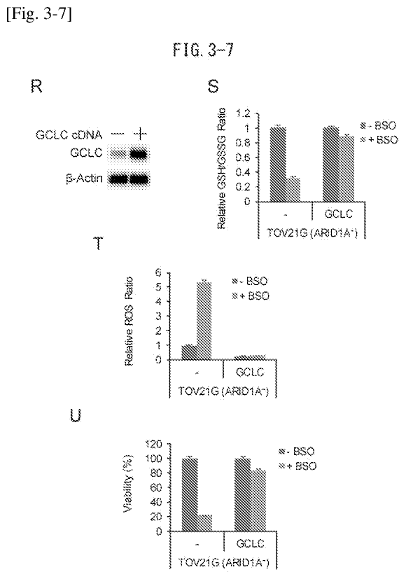

GSH is synthesized from cysteine, glutamate, and glycine via the actions of multiple metabolic factors (FIG. 3-1 A). To identify therapeutic targets against ARID1A-deficient cancers in the GSH metabolic pathway, GSH metabolic pathway genes were screened for a synthetic lethal partnership with ARID1A. Subsequent analyses revealed that knockdown of GCLC, GSS, and SLC7A11 significantly and specifically suppressed the growth of ARID1A-deficient cancer cells, in association with a decrease in the GSH level and an increase in the ROS level (FIGS. 3-2 B to 3-2 D). On the other hand, even simultaneous knockdown of functional paralog gene products such as GLS and GLS2, SLC1A4 and SLC1A5, and IDH1 and IDH2, which are considered to play redundant roles, did not affect the growth of ARID1A-deficient cancer cells (FIG. 3-2 B). Furthermore, knockdown of anti-ROS gene products belonging to the TrxR and superoxide dismutase (SOD) families did not markedly affect cell growth, the GSH level, or the ROS level, even when gene products from multiple families were simultaneously knocked down (Figures S3-1 A to S3-1 C). Next, known inhibitors of GSH metabolic factors were screened for their selectivity for ARID1A-deficient cancers. ARID1A-deficient TOV21G ovarian cancer cells were more sensitive than ARID1A-proficient RMG-I ovarian cancer cells to the GCLC inhibitor buthionine sulfoximine (BSO) and the SLC7A11 inhibitor sulfasalazine, but not to the GLS inhibitor compound 968 or the G6PD inhibitor 6-aminonicotinamide (6-AN) (FIG. 3-3 E), consistent with the knockdown experiments. Similar results were obtained using ARID1A-KO and ARID1A-WT cells (Figure S3-1 D). Knockdown of GCLC, GSS, or SLC7A11 inhibited the growth of ARID1A-deficient OVISE ovarian cancer cells, in association with a decrease in the GSH level and an increase in the ROS level (Figures S3-2 E to S3-3 J). The decrease in GSH level, the increase in ROS level, and the inhibition of cell growth caused by knockdown of GCLC and SLC7A11 in ARID1A-deficient TOV21G cells were rescued by stable expression of the ARID1A cDNA (Figures S3-3 K to S3-4 O), confirming that ARID1A deficiency is responsible for the sensitivity to depletion of those genes.

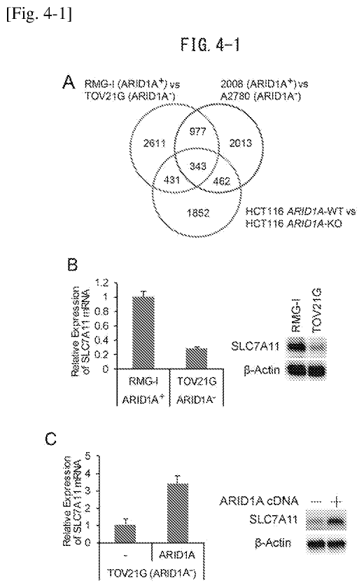

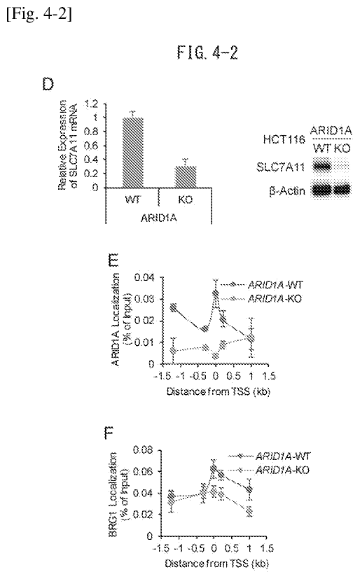

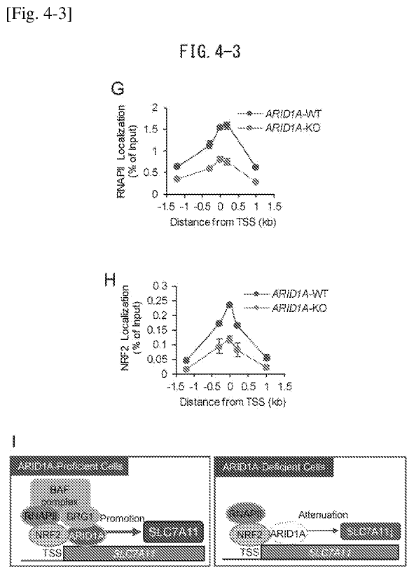

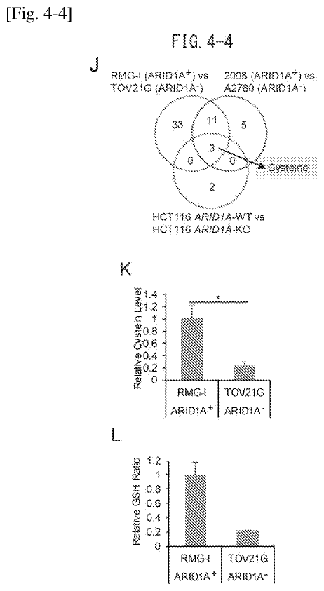

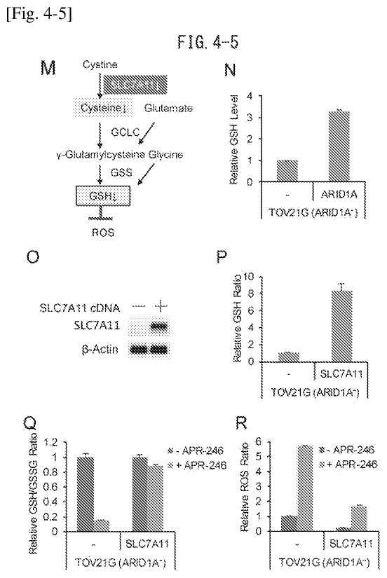

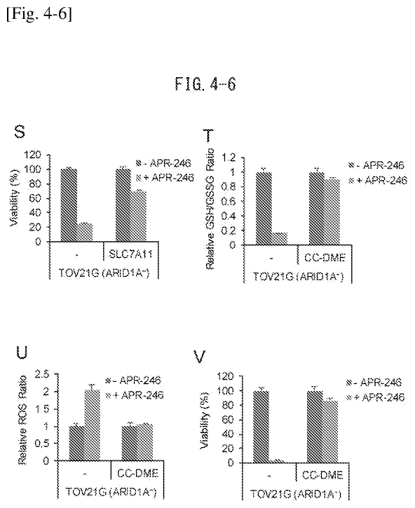

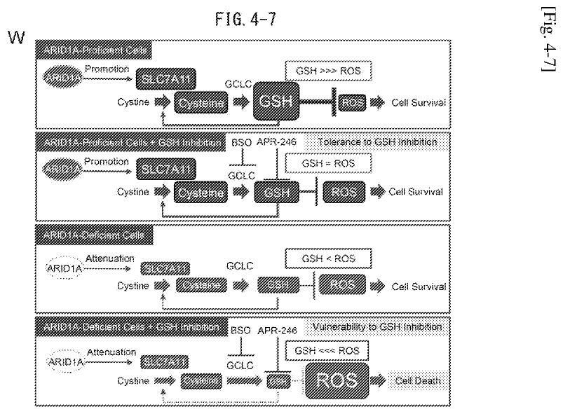

Among the three GSH synthesis pathway enzymes listed above, we focused on GCLC because it is a rate-limiting enzyme (i.e., possibly a druggable target) in GSH synthesis and its transient knockdown markedly inhibited the growth of ARID1A-deficient cancer cells (Figure S3-2 H). In fact, stable shRNA-mediated knockdown of GCLC induced by treatment with doxycycline (Dox) selectively inhibited the growth of ARID1A-deficient cancer cells (FIGS. 3-4 F to 3-4 G). GSH and ROS levels were significantly decreased and increased, respectively, in ARID1A-deficient cancer cells but not ARID1A-proficient cells (FIGS. 3-4 H to 3-4 I). Similar results were obtained using ARID1A-KO and ARID1A-WT cells (Figures S3-4 P to S3-4 S). The lethality of GCLC depletion in ARID1A-deficient cancer cells was validated by measuring cell survival in colony formation assays (FIG. 3-5 J, Figures S3-5 T to S3-5 U). Ectopic expression of GCLC abrogated the decrease in the GSH level upon knockdown of GCLC in ARID1A-deficient cell lines and ARID1A-KO cells (FIGS. 3-5 K to 3-5 L, Figures S3-5 V to S3-5 W). In addition, ectopic GCLC expression abrogated ROS production and subsequent cell death upon GCLC knockdown (FIGS. 3-5 M to 3-5 N, Figures S3-5 X to S3-5 Y). Taken together, these data indicate that GCLC is a synthetic lethal target in ARID1A-deficient cancer cells.

To determine whether GCLC is a druggable target, we examined the sensitivities of four ARID1A-proficient and six ARID1A-deficient cancer cell lines to the GCLC inhibitor BSO. ARID1A-deficient cancer cell lines were selectively sensitive to BSO (FIG. 3-6 O, Figure S4-1 A). Colony formation assays demonstrated that BSO selectively reduced survival and increased apoptosis of ARID1A-deficient cancer cell lines and ARID1A-KO cells (FIGS. 3-6 P to 3-6 Q, Figures S4-1 B to S4-2 E). BSO treatment decreased the GSH level, which was associated with increased ROS levels and NOXA expression, and this effect was more prominent in ARID1A-deficient cancer cells (Figures S4-2 F to S4-2 J). Stable expression of ARID1A abrogated the BSO-induced decrease in GSH, increase in ROS, and inhibition of cell growth of ARID1A-deficient cancer cells (Figures S4-3 K to S4-3 M), indicating that the observed responses to BSO are dependent on ARID1A. The BSO-induced decrease in GSH in ARID1A-deficient cancer cells was attenuated by the GSH compensator GSH-MEE, but not by the ROS scavenger NAC (Figure S4-4 N). The APR-246-induced decrease in GSH in ARID1A-deficient cancer cells was suppressed by both GSH-MEE and NAC (FIG. 2-4 L, Figure S2-2 D), consistent with the fact that NAC inhibits ROS and APR-246 activity by forming adducts (See, Lambert et al., Cancer Cell 15, 376-388, 2009). Both GSH-MEE and NAC abrogated the BSO-induced increase in ROS and inhibition of cell growth (Figures S4-4 O to S4-5 P). These results indicate that even when GSH production is decreased by BSO treatment, NAC and GSH-MEE restore cell viability by scavenging excessive ROS and compensating for GSH, respectively. Ectopic expression of GCLC suppressed all the effects of BSO and APR-246 in ARID1A-deficient cancer cells, including the decrease in GSH, the increase in ROS, and the inhibition of cell growth (FIGS. 3-7 R to 3-7-U, Figures S4-5 Q to S4-6 S). These results strongly suggest that GCLC is a druggable target in ARID1A-deficient cancer cells.

### Example 4: Vulnerability of ARID1A-Deficient Cancer Cells to GSH Inhibition is Caused by Decreased GSH Synthesis Due to Impaired SLC7A11 Expression

ARID1A-deficient cancer cells were sensitive to GSH inhibition compared with ARID1A-proficient cells. We therefore hypothesized that ARID1A regulates transcription of genes encoding components of the GSH synthesis pathway. To investigate this, a genome-wide expression analysis of a panel of ARID1A-proficient and ARID1A-deficient cancer cells was performed. In total, 343 genes whose expression levels were consistently more than 1.5-fold lower in ARID1A-deficient cancer cells were identified (FIG. 4-1 A). Notably, this gene set included only one GSH metabolic pathway gene SLC7A11. Expression of SLC7A11 mRNA and protein was lower in ARID1A-deficient cancer cells than in ARID1A-proficient cancer cells (FIG. 4-1 B, Figures S5-1 A to S5-1 C). Stable expression of ARID1A in ARID1A-deficient cancer cells restored expression of SLC7A11 mRNA and protein (FIG. 4-1 C). In addition, knockout of ARID1A decreased expression of SLC7A11 (FIG. 4-2 D). To determine the mechanism underlying the impaired induction of SLC7A11 in ARID1A-KO cells, the involvement of ARID1A in the transcriptional upregulation of SLC7A11 was investigated. A chromatin immunoprecipitation (ChIP) assay revealed that ARID1A localized at the transcription start site (TSS) of SLC7A11 in ARID1A-WT cells (FIG. 4-2 E). Likewise, BRG1, the catalytic subunit of the SWI/SNF chromatin remodeling complex (the BAF complex) that contains ARID1A, localized at the TSS of SLC7A11 in ARID1A-WT cells (FIG. 4-2 F). Localization of ARID1A and BRG1 at the TSS was impaired in ARID1A-KO cells (FIGS. 4-2 E to 4-2 F). Localization of RNA polymerase II (RNAPII) at the TSS was also markedly impaired in ARID1A-KO cells (FIG. 4-3 G). Localization of NRF2, a transcription factor regulating SLC7A11 expression (See, Gorrini et al., Nat Rev Drug Discov 12, 931-947, 2013), at the TSS was also markedly impaired in ARID1A-KO cells (FIG. 4-3 H), whereas expression of NRF2 itself was not affected by knockout of ARID1A (Figure S5-2 D). Ectopic expression of NRF2 in ARID1A-KO cells restored expression of SLC7A11 mRNA and protein (Figures S5-2 D to S5-2 E), restored the APR-246-induced decrease in growth and survival, and abrogated the APR-246-induced decrease in GSH and increase in ROS (Figures S5-2 F to S5-3 I). These results strongly suggest that ARID1A promotes, but is not essential for, NRF2-mediated transcriptional expression of SLC7A11 through chromatin remodeling by the BAF complex. In other words, ARID1A deficiency causes attenuation of SLC7A11 expression (FIG. 4-3 I).

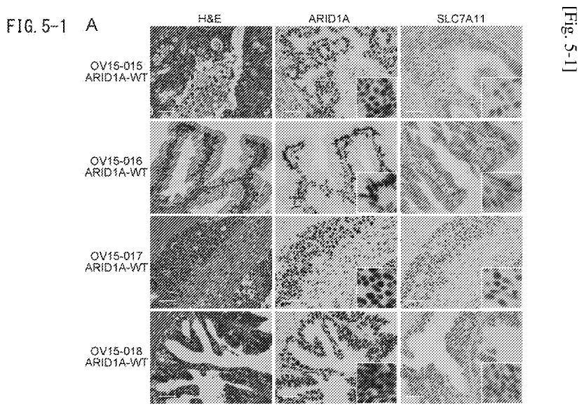

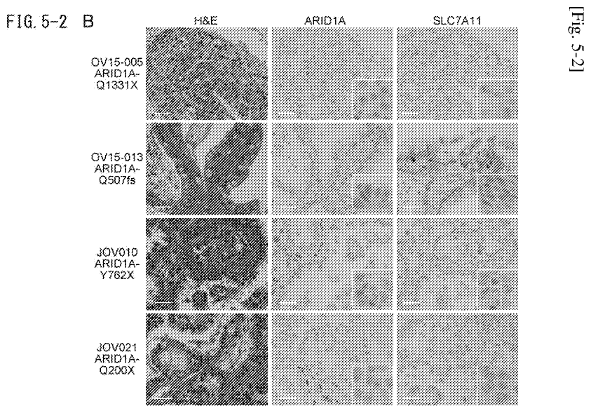

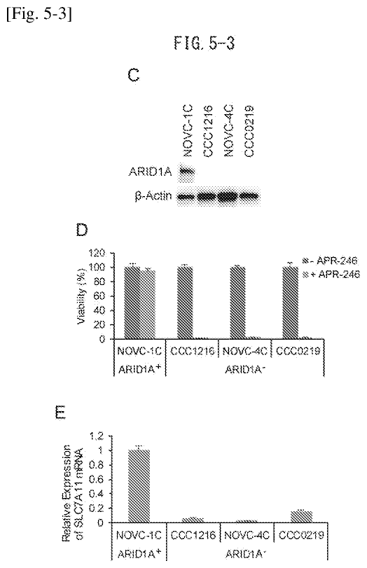

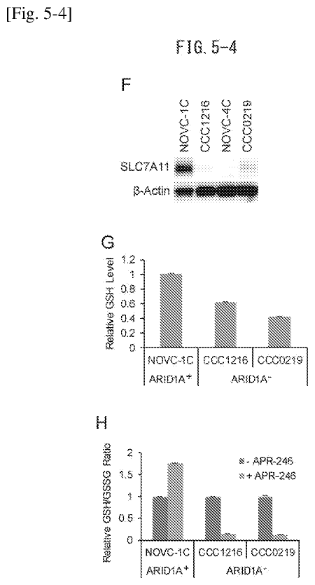

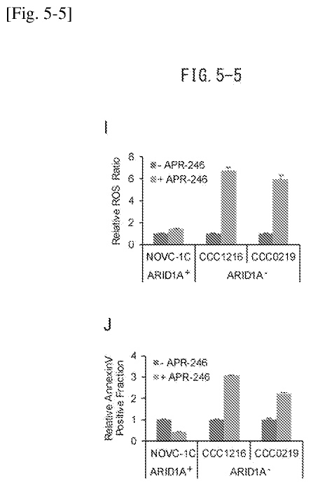

SLC7A11 encodes a subunit of the cystine/glutamate transporter XCT. Cystine is taken up into cells through the XCT transporter and is then metabolized into two molecules of cysteine, which is essential for GSH synthesis. To investigate the effects of downregulation of SLC7A11 caused by ARID1A deficiency, we screened metabolites whose levels were decreased in the three pairs of ARID1A-proficient and ARID1A-deficient cells using gas chromatography/mass spectrometry (GCMS), which is able to detect 475 metabolites (including cysteine, glutamate, and glycine, but not GSH) (FIG. 4-4 J). Cysteine was identified as a metabolite that was more than 2-fold lower in ARID1A-deficient cells than in ARID1A-proficient cells (FIGS. 4-4 J to 4-4 K, Figure S5-3 J), suggesting that SLC7A11 downregulation caused a decrease in intracellular cysteine levels due to impaired uptake of cystine into cells. Consistent with the decreased level of cysteine in ARID1A-deficient cells, the basal GSH level was lower in ARID1A-deficient cells than in ARID1A-proficient cells (FIGS. 4-4 L to 4-5 M, Figures S5-4 K to S5-4 L). Reflecting the increased GSH level, the ROS level was higher in ARID1A-deficient cells than in ARID1A-proficient cells (Figures S5-5 M to S5-5 O). As shown in FIG. 4-1 C, stable expression of ARID1A in ARID1A-deficient cancer cells increased SLC7A11 expression, which was associated with an increase in the GSH level and a decrease in the ROS levels (FIG. 4-5 N, Figure S5-6 P). These results indicate that ARID1A affects the balance between the basal levels of GSH and ROS through expression of SLC7A11. Consistently, forced expression of SLC7A11 in ARID1A-deficient cancer cells increased the basal GSH levels, which was associated with decreased basal ROS levels (FIGS. 4-5 O to 4-5 P, Figure S5-6 Q). Similar results were obtained by stable expression of GCLC in ARID1A-deficient cancer cells (Figures S5-6 R to S5-6 S). Overexpression of SLC7A11 abrogated the APR-246- and BSO-induced decreases in the GSH level and increases in the ROS level, and restored the viability of ARID1A-deficient cancer cells (FIGS. 4-5 Q to 4-6 S, Figures S5-7 T to S5-7 V).

The decrease in GSH, increase in ROS, and decrease in viability in APR-246-treated ARID1A-deficient cancer cells were completely suppressed by co-treatment with the cystine compensator cystine dimethyl ester (CC-DME), a cell-permeable version of cystine (See, Steinherz et al., Proc Natl Acad Sci USA 79, 4446-4450, 1982) (FIGS. 4-6 T to 4-6 V). These data indicate that a cysteine shortage due to impaired SLC7A11 expression in ARID1A-deficient cancer cells is the cause of their vulnerability to inhibition of the GSH metabolic pathway. Collectively, these results lead us to propose the following mechanism (FIG. 4-3 I, 4-7 W). ARID1A facilitates recruitment of the SWI/SNF chromatin-remodeling complex to the TSS of SLC7A11, and the resultant remodeling initiates the transcription of SLC7A11 by NRF2 and RNAPII. Enhanced cystine uptake by SLC7A11 upregulates GSH synthesis via GCLC, maintaining the homeostatic balance between GSH and ROS and making cells tolerant of GSH inhibition. However, in the absence of ARID1A, impaired recruitment of the SWI/SNF chromatin-remodeling complex to the TSS of SLC7A11 attenuates recruitment of NRF2 and RNAPII to this site, resulting in impaired transcription of SLC7A11. The resultant decrease in cystine uptake impairs GSH synthesis. In addition, impairment of the feedback function of GSH to produce cysteine from cystine would further decrease the intracellular level of cysteine and eventually lead to shortage of GSH due to perturbed homeostasis of the antioxidant system. The decrease in the basal GSH level makes ARID1A-deficient cancer cells vulnerable to inhibition of the GSH metabolic pathway.

### Example 5: Clinical Relevance of GSH-Targeting Therapy for ARID1A-Deficient Cancers

To further address the clinical relevance of this hypothesis, we examined expression of ARID1A and SLC7A11 by immunohistochemical staining of tumor specimens surgically obtained from 11 ovarian cancer patients (Figure S6-1 A). All four ARID1A wild-type specimens examined showed high expression of both ARID1A and SLC7A11 (FIG. 5-1 A), while all four of the ARID1A-mutated specimens lacking ARID1A expression showed low levels of SLC7A11 expression (FIG. 5-2 B). The remaining three ARID1A-mutated specimens, which retained ARID1A expression, also retained SLC7A11 expression (Figures S6-1 A, S6-1 B). Taken together, reduced/absent ARID1A expression due to ARID1A gene mutations correlated with decreased SLC7A11 expression in clinically obtained tumor specimens.

Patient-derived cancer cells (PDCs) are often serve as a tool to obtain proof-of-concept for cancer therapy. Thus, PDCs from four other ovarian cancer patients, three cases lacked ARID1A whereas the remaining case retained ARID1A expression, were cultured (FIG. 5-3 C). Consistent with the results obtained using cancer cell lines, ARID1A-deficient PDCs were more sensitive to APR-246 and BSO than ARID1A-positive PDCs (FIG. 5-3 D, Figure S6-2 C). In addition, expression of SLC7A11 mRNA and protein in ARID1A-deficient PDCs was low, which is in agreement with the observations in tumor specimens described above (FIGS. 5-3 E to 5-4 F). Basal GSH levels were lower in ARID1A-deficient PDCs than in ARID1A-proficient PDCs (FIG. 5-4 G), while basal ROS levels were higher (Figure S6-2 D). APR-246 treatment decreased the GSH levels in ARID1A-deficient PDCs and increased the levels of ROS and apoptosis (FIGS. 5-4 H to 5-5 J, Figure S6-2 E).

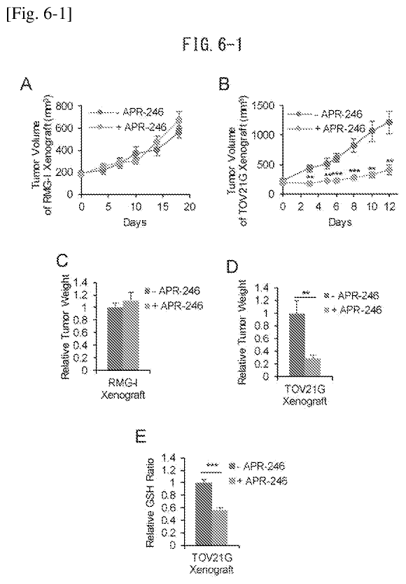

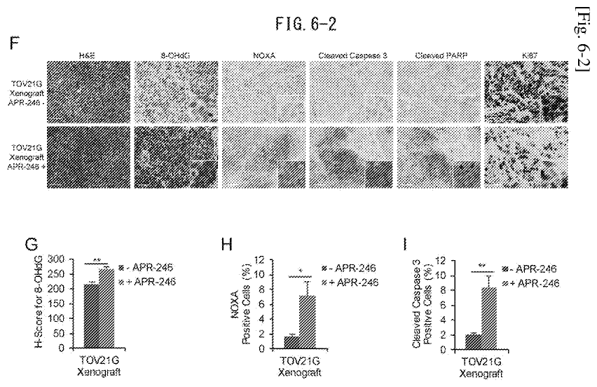

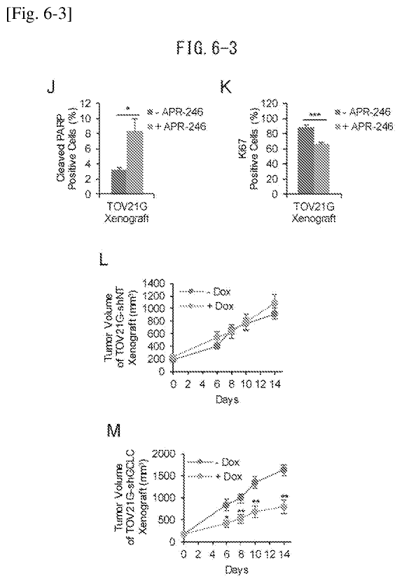

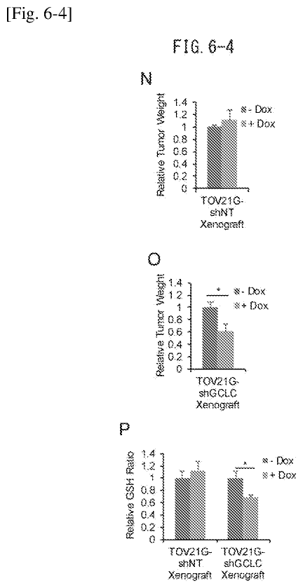

Next, APR-246 was tested for its ability to suppress the growth of OCCC tumor xenografts in vivo. After the tumor formation, mice were treated with APR-246 or vehicle. APR-246 treatment significantly suppressed the growth of ARID1A-deficient TOV21G OCCC xenografts, but not that of ARID1A-proficient RMG-I OCCC xenografts (FIGS. 6-1 A to 6-1 B). Consistently, tumor weight was reduced by APR-246 only in TOV21G xenografts (FIGS. 6-1 C to 6-1 D). Treatment of mice with APR-246 did not cause significant loss of body weight, indicating that APR-246 has minimal adverse effects (Figure S6-3 F), consistent with the results of a phase I clinical trial (See, Lehmann et al., J Clin Oncol 30, 3633-3639, 2012). Notably, the GSH level in TOV21G xenograft cells decreased after treatment with APR-246 (FIG. 6-1 E). In addition, APR-246 increased the level of the oxidative stress marker 8-hydroxy-2′-deoxyguanosine (8-OHdG) markedly, indicating that APR-246 increased the ROS levels in TOV21G xenografts in vivo (FIGS. 6-2 F to 6-2 G). In addition, APR-246 treatment markedly increased expression of the apoptotic markers NOXA, cleaved PARP, and cleaved caspase-3, and decreased expression of the cell proliferation marker Ki67, indicating that apoptosis was also induced by treatment with APR-246 in vivo (FIGS. 6-2 F, 6-2 H to 6-2 G). Consistently, the APR-246 analog PRIMA-1 treatment also significantly suppressed the growth of xenografts in ARID1A-deficient OVISE OCCC cells, again without causing significant weight loss (Figures S6-3 G to S6-3 H). In addition, treatment with the GCLC inhibitor BSO markedly suppressed xenograft growth, resulting in reduced tumor weight without loss of body weight (Figures S6-3 I to S6-3 K). The present results obtained using drugs inhibiting GSH function via different mechanisms demonstrate the utility of targeting GCLC in ARID1A-deficient OCCC.

To further validate GCLC as a therapeutic target, the effects of GCLC depletion were assessed in an ARID1A-deficient OCCC tumor xenograft model. TOV21G OCCC cells carrying non-targeting (shNT) or GCLC-targeting (shGCLC) shRNAs were injected into mice. GCLC expression in TOV21G-shGCLC cells was conditionally reduced by Dox (FIG. 3-4 F). When mice were fed with Dox to induce GCLC depletion after tumor formation, the growth of TOV21G-shGCLC xenografts was suppressed significantly, resulting in reduced tumor weight, whereas the growth of TOV21G-shNT xenografts was not affected (FIGS. 6-3 L to 6-4 O). Knockdown of GCLC in TOV21G-shGCLC xenografts, but not in TOV21G-shNT xenografts, was confirmed in tumors from mice treated with Dox (Figures S6-4 L to S6-4 M). The GSH level in TOV21G-shGCLC xenograft cells decreased in vivo after treatment with Dox (FIG. 6-4 P). Similar results were also obtained when mice were fed Dox immediately after inoculation of cells (before tumor formation) (Figures S6-4 N to S6-5 R). As with APR-246 treatment of TOV21G xenografts, Dox-mediated knockdown of GCLC in TOV21G-shGCLC xenografts markedly increased the level of the oxidative stress marker 8-0HdG; increased the levels of the apoptosis markers NOXA, cleaved caspase-3, and cleaved PARP; and decreased the level of the cell proliferation maker, Ki67. These results indicate that ROS levels and apoptosis were increased by inhibition of GCLC in vivo (Figure S6-6 S). Taken together, we conclude that GCLC is a therapeutic target in ARID1A-deficient OCCC.

### Example 6: Selection of ARID1A-Deficient and -Proficient Patient-Derived Cells Established from the Ascites of Patients with Diffuse-Type Gastric Cancer

Of >100 patient-derived cells (PDCs) obtained from the ascites of 65 patients with diffuse-type gastric cancer, we selected 13 cell types (NSC-4X1a, -7C, -14C, -20C, -22C, -34C, -48CA, -58C, -64C, -65C, -67C, -68C, and -70C) showing adherent cell growth and lower dispersion in the drug-sensitivity test than floating cells. ARID1A protein expression was investigated by immunoblot analysis. Eight PDCs were selected for further analysis based on whole exome data. Of these eight PDCs, four (NSC-7C, -58C, -65C, and -67C) lacked ARID1A protein expression (ARID1A-deficient: ARID1A-) and four (NSC-48C, -64C, -68C, and -70C) retained ARID1A protein expression (ARID1A-proficient: ARID1A+) (FIG. 7A). SLC7A11 expression was lower in ARID1A-deficient PDCs than ARID1A-proficient PDCs (FIG. 7A), consistent with the pattern observed in ovarian cancer (See, H. Ogiwara et al., Cancer Cell 35 (2019) 177-190 e178, the content of the document is the same as U.S. Provisional Application Ser. No. 62/785,458). Consistent with ARID1A protein levels, three (NSC-7C, -58C, and -67C) of four ARID1A-deficient PDCs had homogeneous frame-shift mutations in the ARID1A gene, and the remaining PDC (NSC-65C) had a homogeneous stop codon mutation (R1461X). The four ARID1A-proficient PDCs had no mutations. Xenograft tumors derived from established PDCs retained the histological properties of diffuse-type gastric cancers (FIG. 7B). Xenograft tumors derived from established PDCs retained the histological properties of diffuse-type gastric cancers. Representative histological data are shown in FIG. 7B.

### Example 7: ARID1A-Deficient Gastric Cancer Cells are Sensitive to GSH Inhibitors

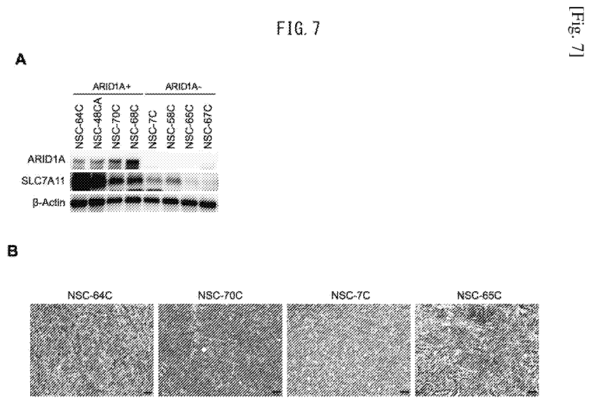

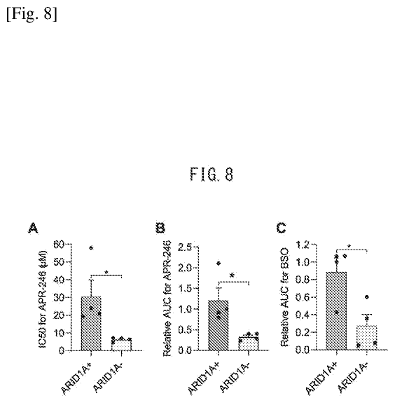

We next examined the sensitivity of ARID1A-deficient gastric cancer cells to GSH inhibitors. The IC50 values for the GSH inhibitor APR-246 were markedly lower in ARID1A-deficient PDCs than in ARID1A-proficient PDCs (FIGS. 8A, 8B). Treatment with BSO, an inhibitor of the GSH synthesis enzyme GCLC, sensitized ARID1A-deficient PDCs more efficiently than ARID1A-proficient PDCs FIG. 8C). These results indicate that sensitivity to APR-246 or BSO is associated with ARID1A deficiency in gastric cancer, which is consistent with the results obtained in ovarian cancer (See, H. Ogiwara et al., Cancer Cell 35 (2019) 177-190 e178). Taken together, these data indicate that GSH inhibition might be a promising strategy for the treatment of diffuse-type gastric cancers with ARID1A-deficiency.

### Example 8: ARID1A-Deficient Gastric Cancer Cells are Vulnerable to GSH Inhibition Due to Low Basal Levels of GSH

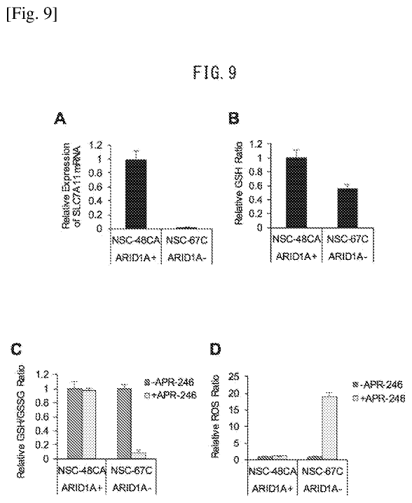

Next, we investigated whether low expression of the SLC7A11 protein in ARID1A-deficient gastric cancers is associated with decreased SLC7A11 transcription. SLC7A11 mRNA levels were lower in ARID1A-deficient than in ARID1A-proficient PDCs (FIGS. 9A, S7A). Since SLC7A11 is required for GSH synthesis by supplying intracellular cysteine, we examined whether SLC7A11 downregulation leads to decreased GSH synthesis. The basal levels of GSH were considerably lower in ARID1A-deficient than in ARID1A-proficient PDCs (FIGS. 9B, S7B). These results indicate that ARID1A-deficiency downregulates SLC7A11 expression and decreases the basal levels of GSH in diffuse-type gastric cancer cells, consistent with the findings in ovarian cancer (See, H. Ogiwara et al., Cancer Cell 35 (2019) 177-190 e178).

APR-246 inhibits GSH activity by reacting with thiol groups (See, B. Tessoulin et al., Blood 124 (2014) 1626-1636). Therefore, we next examined whether APR-246 preferentially inhibits GSH in ARID1A-deficient cancer cells. APR-246 treatment markedly decreased GSH levels in ARID1A-deficient PDCs and not in ARID1A-proficient PDCs (FIGS. 9C, S7C). Consistent with the antioxidant activity of GSH, ROS levels were increased more markedly in ARID1A-deficient than in ARID1A-proficient PDCs (FIGS. 9D, S7D). These results indicate that the excessive increase of oxidative stress induced by GSH inhibitors in ARID1A-deficient cells decreased cell viability.

### Example 9: Vulnerability of ARID1A-Deficient Gastric Cancer Cells to GSH Inhibition is Caused by Decreased GSH Synthesis Due to Diminished SLC7A11 Expression

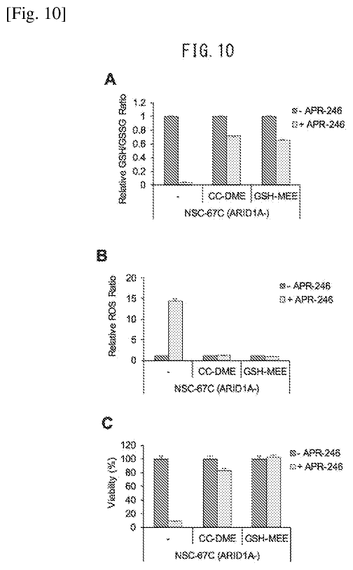

We next examined whether the vulnerability of ARID1A-deficient cancer cells is related to cysteine shortage and consequent GSH shortage. The APR-246-induced GSH decrease, ROS increase, and cell death in ARID1A-deficient cancer cells were markedly suppressed by co-treatment with the cystine compensator cystine dimethyl ester (CC-DME) or the GSH compensator glutathione monoethyl ester (GSH-MEE), cell-permeable versions of cystine and GSH, respectively, suggesting that these cell-permeable metabolites were able to compensate for impairment of cystine uptake due to diminished SLC7A11 expression (FIGS. 10A to 10C). GSH is synthesized from cysteine, glutamate, and glycine. SLC7A11 contributes to GSH synthesis by transporting cysteine into the cell. Therefore, we next examined the sensitivity to erastin, an SLC7A11 inhibitor (See, B. Daher et al., Cancer Res 79 (2019) 3877-3890), in ARID1A-deficient PDCs. The IC50 values for erastin were markedly lower in ARID1A-deficient PDCs than in ARID1A-proficient PDCs (Figure S8A). Erastin treatment markedly decreased GSH levels in ARID1A-deficient PDCs and not in ARID1A-proficient PDCs (Figure S8B). These data indicate that a cysteine shortage and consequent GSH shortage secondary to diminished SLC7A11 expression in ARID1A-deficient cancer cells are the cause of their sensitivity to GSH inhibition.

### Example 10: ARID1A-Deficient Uterine Cancer Cells are Sensitive to GSH Inhibitors

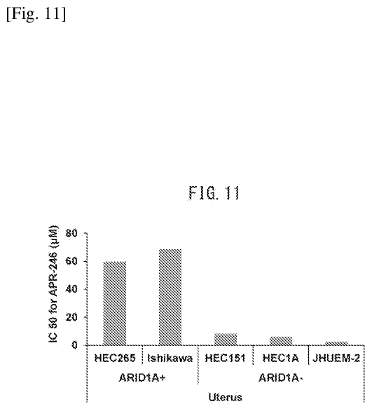

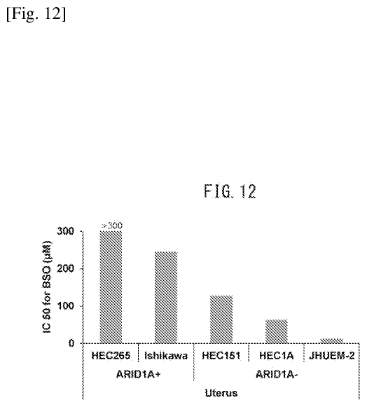

We examined the sensitivity of ARID1A-deficient uterine cancer cells to GSH inhibitors. The IC50 values for the GSH inhibitor APR-246 were markedly lower in three ARID1A-deficient cell lines than in two ARID1A-proficient cell lines (FIG. 11). Treatment with BSO, an inhibitor of the GSH synthesis enzyme GCLC, sensitized ARID1A-deficient cell lines more efficiently than ARID1A-proficient cell lines (FIG. 12). These results indicate that sensitivity to APR-246 or BSO is associated with ARID1A deficiency in urerine cancer, which is consistent with the results obtained in ovarian cancer (See, H. Ogiwara et al., Cancer Cell 35 (2019) 177-190 e178). Taken together, these data indicate that GSH inhibition might be a promising strategy for the treatment of uterine cancers with ARID1A-deficiency.

### Example 11: ARID1A-Deficient Bile Duct Cancer Cells are Sensitive to GSH Inhibitors

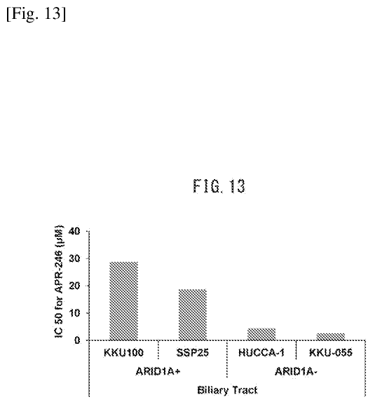

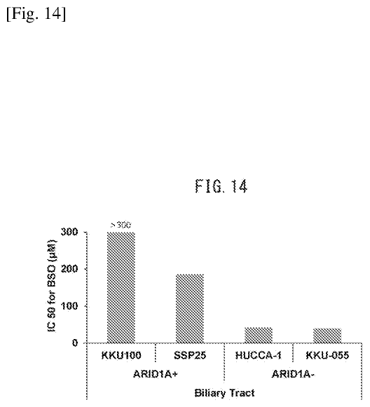

We examined the sensitivity of ARID1A-deficient bile duct cancer cells to GSH inhibitors. The IC50 values for the GSH inhibitor APR-246 were markedly lower in two ARID1A-deficient cell lines than in two ARID1A-proficient cell lines (FIG. 13). Treatment with BSO, an inhibitor of the GSH synthesis enzyme GCLC, sensitized ARID1A-deficient cell lines more efficiently than ARID1A-proficient cell lines (FIG. 14). These results indicate that sensitivity to APR-246 or BSO is associated with ARID1A deficiency in bile duct cancer, which is consistent with the results obtained in ovarian cancer (See, H. Ogiwara et al., Cancer Cell 35 (2019) 177-190 e178). Taken together, these data indicate that GSH inhibition might be a promising strategy for the treatment of bile duct cancers with ARID1A-deficiency.

### Example 12: Various SWI/SNF Complexes Deficient Cancer Cell Lines are Sensitive to GSH Inhibitors

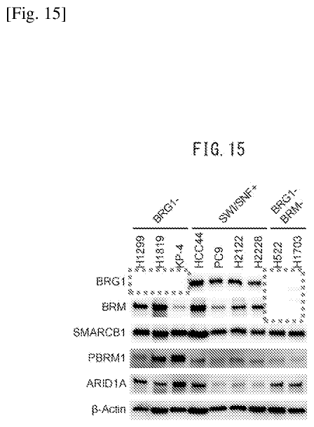

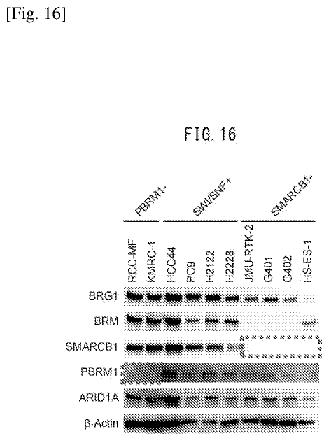

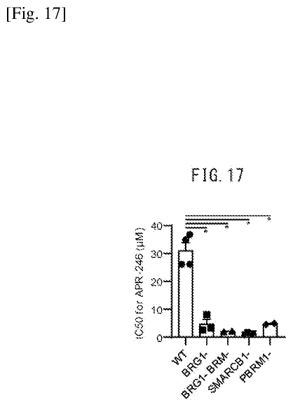

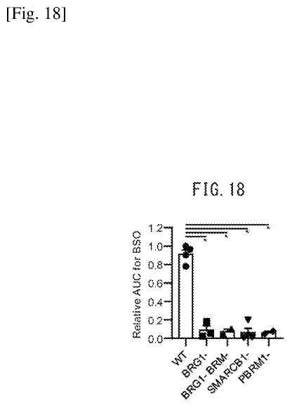

We examined the sensitivity of various SWI/SNF complexes (SMARCA2 (BRM), SMARCA4 (BRG1, BAF190), SMARCB1 (BAF47, hSNF5, INI1), PBRM1) deficient cancer cells to GSH inhibitors. The IC50 values for the GSH inhibitor APR-246 were markedly lower in these SWI/SNF complexes deficient cell lines than in wild-type cell lines (FIGS. 15, 16, 17). Treatment with BSO, an inhibitor of the GSH synthesis enzyme GCLC, sensitized these SWI/SNF complexes deficient cell lines more efficiently than these SWI/SNF complexes proficient cell lines (FIGS. 15, 16, 18). These results indicate that sensitivity to APR-246 or BSO is associated with these SWI/SNF complexes (i.e., SMARCA2, SMARCA4, SMARCB1, PBRM1) deficiency in various cancers. Taken together, these data indicate that GSH inhibition might be a promising strategy for the treatment of various cancers with these SWI/SNF complexes deficiency.

### Example 13: Vulnerability of SWI/SNF Complexes-Deficient Cancer Cells to GSH InHibition is Caused by Decreased GSH Synthesis Due to Impaired SLC7A11 Expression

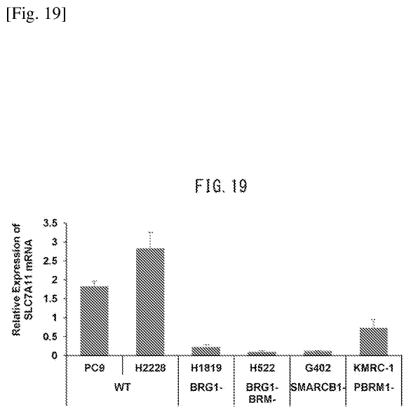

SWI/SNF complexes except for ARID1A (i.e, SMARCA2, SMARCA4, SMARCB1, PBRM1) -deficient cancer cells were similarly sensitive to GSH inhibition compared with these SWI/SNF complexes proficient cells. To investigate this, expression of SLC7A11 mRNA was lower in these SWI/SNF complexes-deficient cancer cells than in various SWI/SNF complexes proficient cancer cells (FIG. 19).

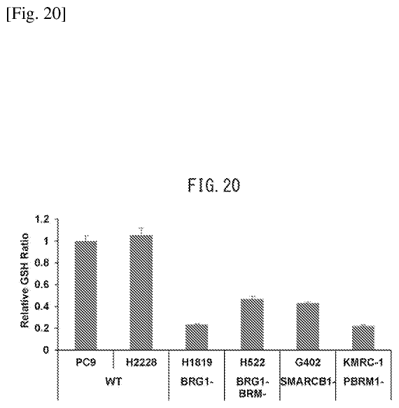

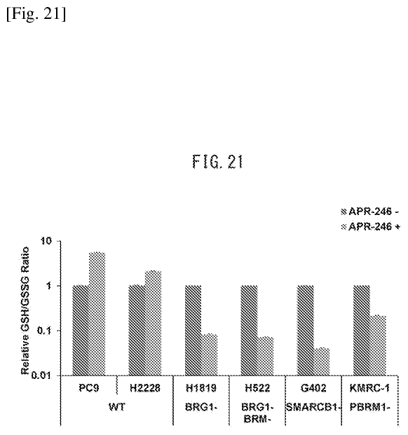

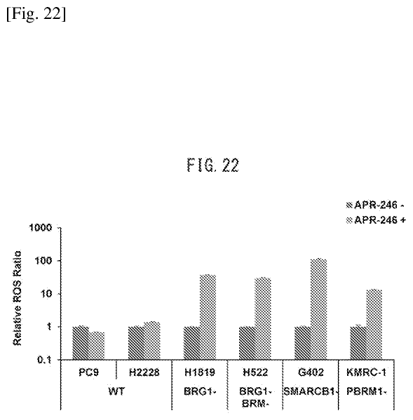

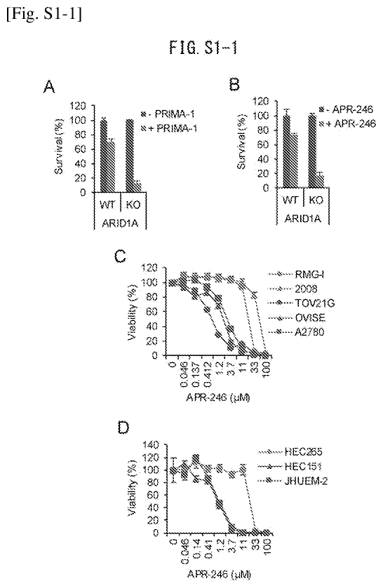

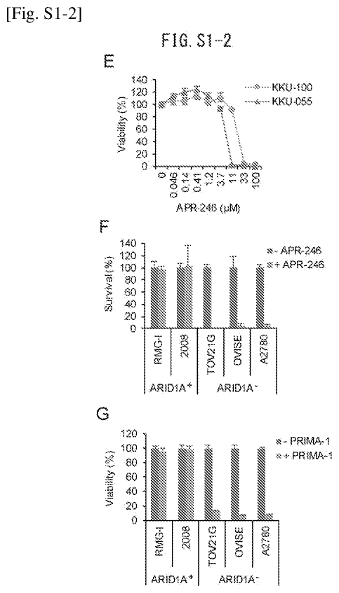

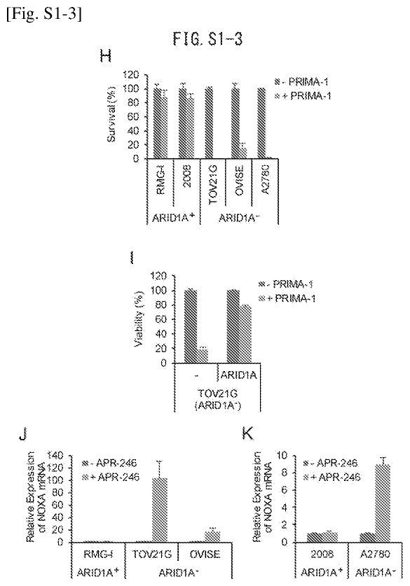

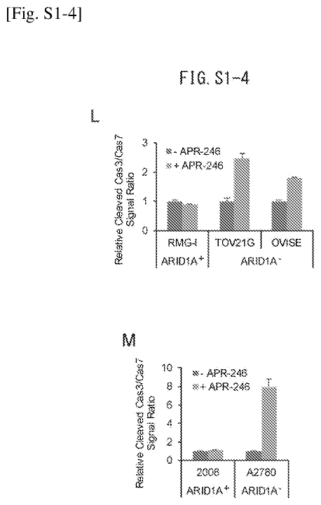

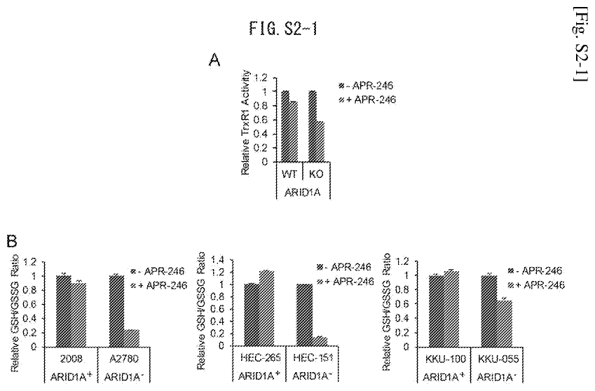

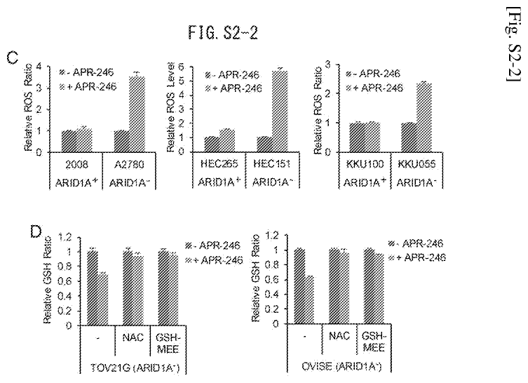

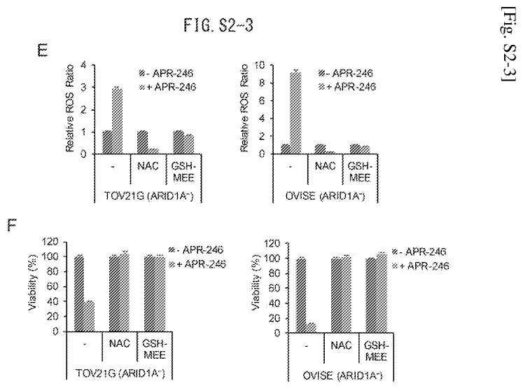

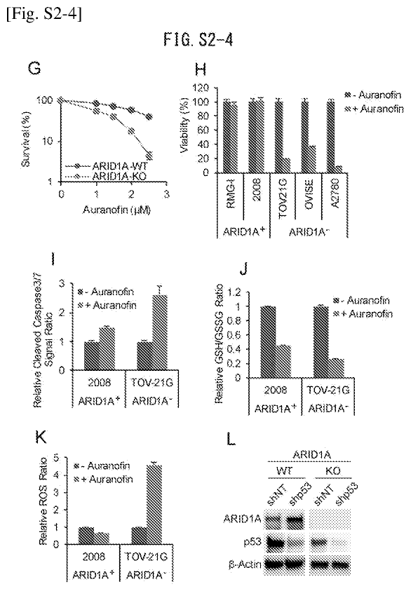

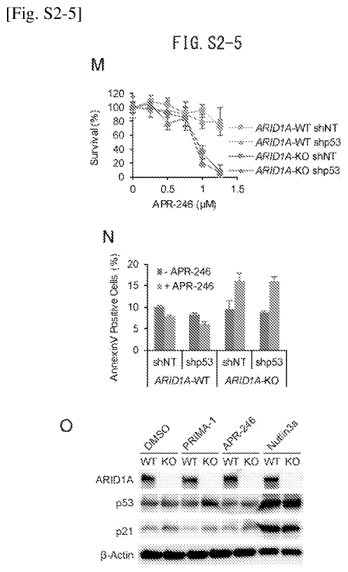

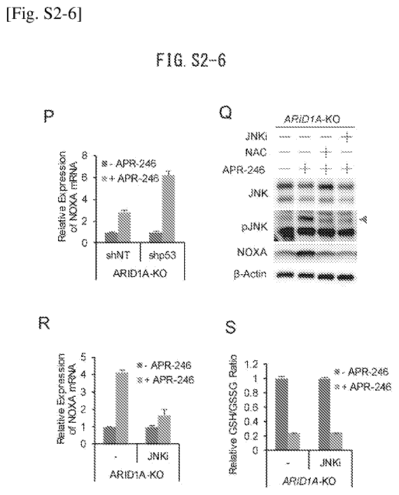

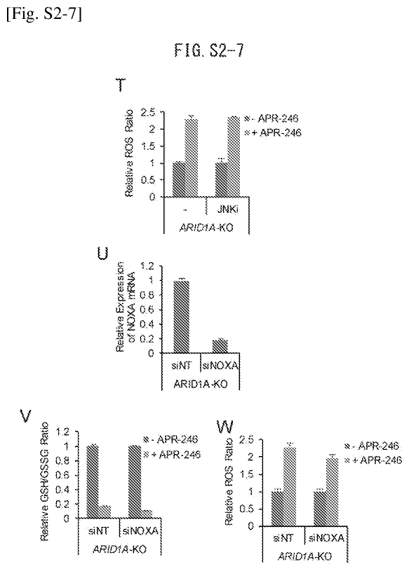

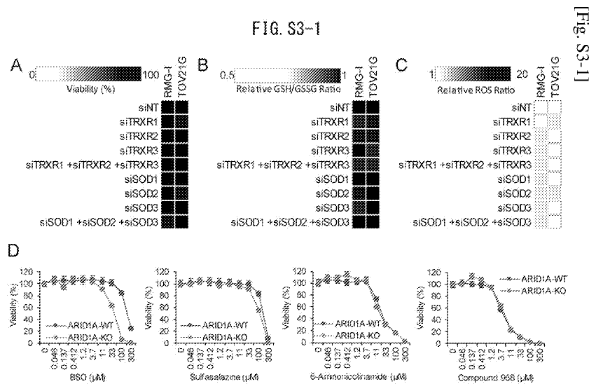

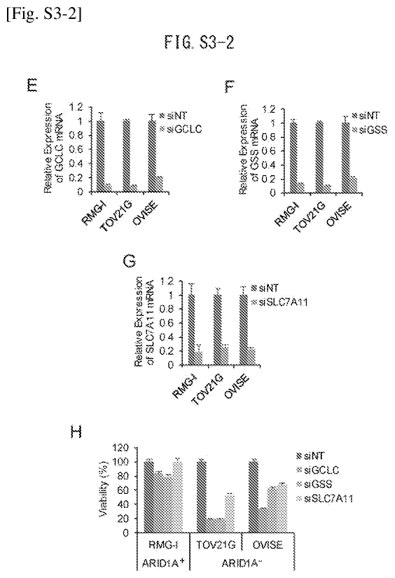

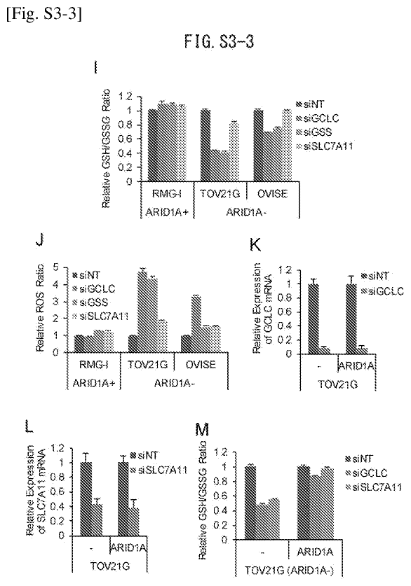

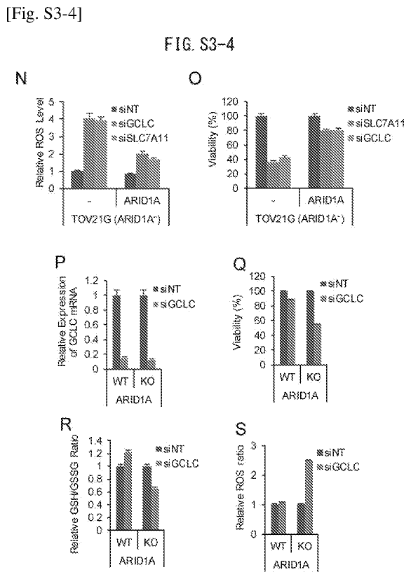

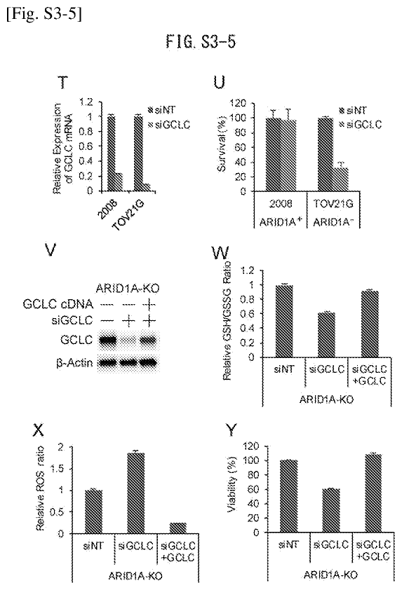

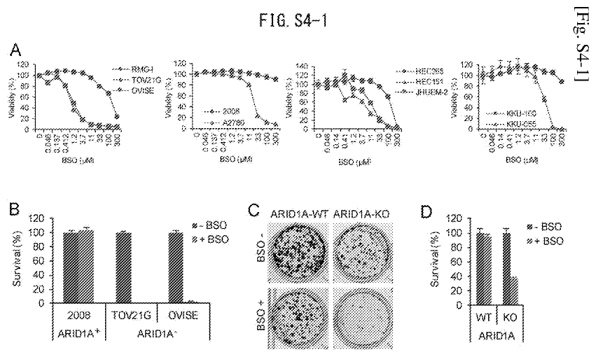

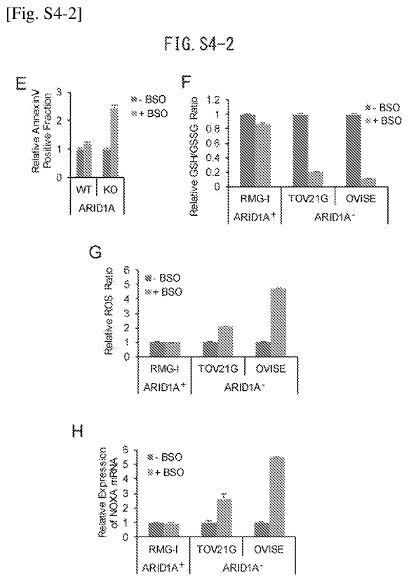

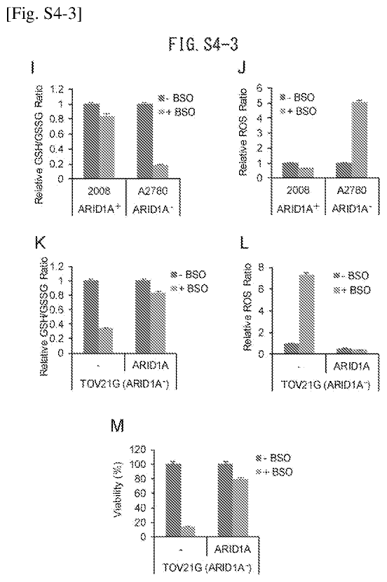

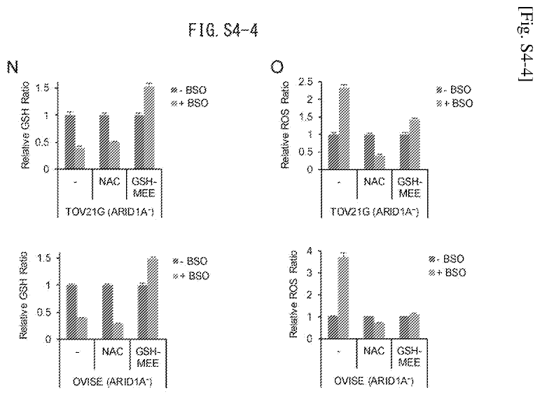

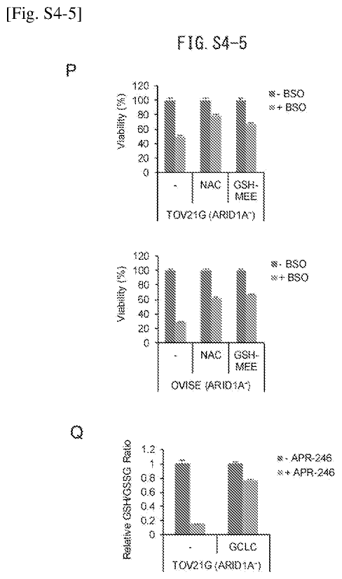

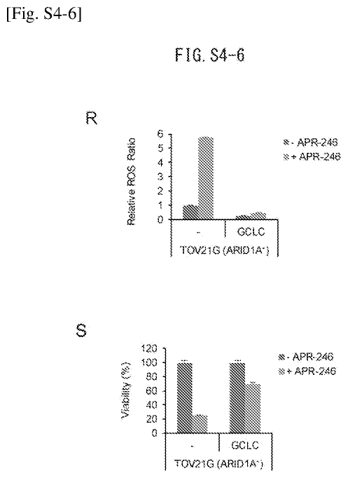

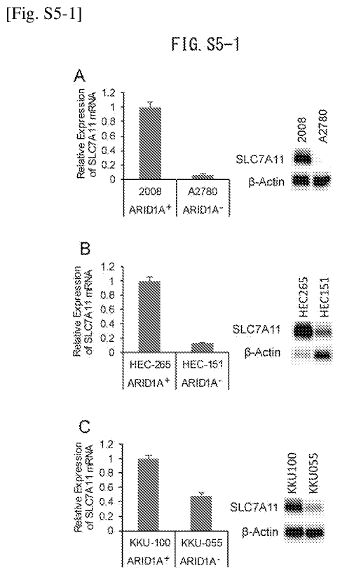

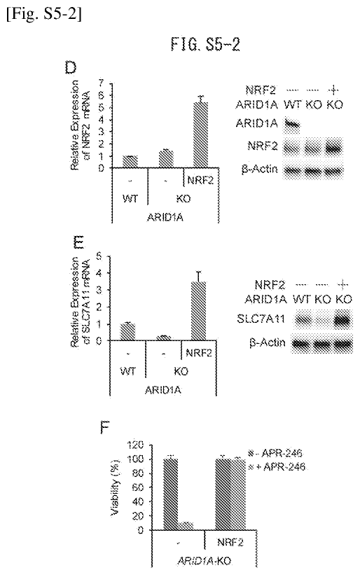

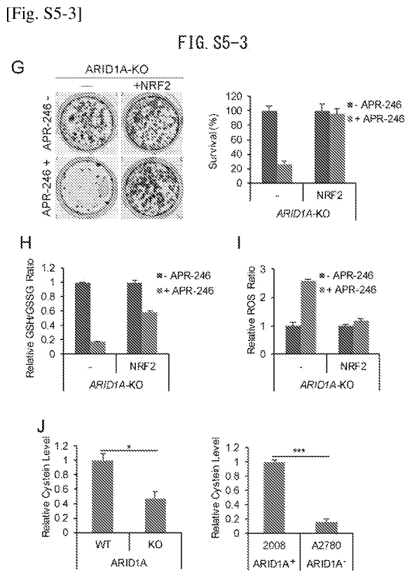

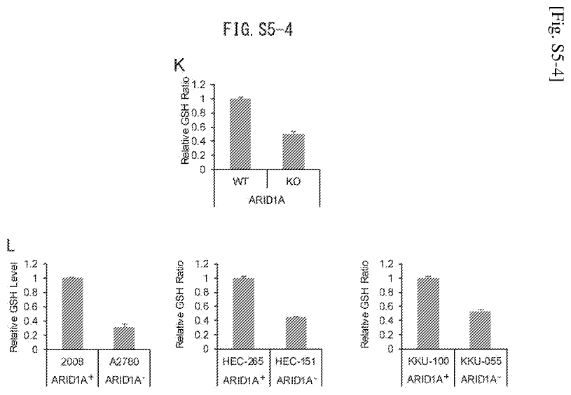

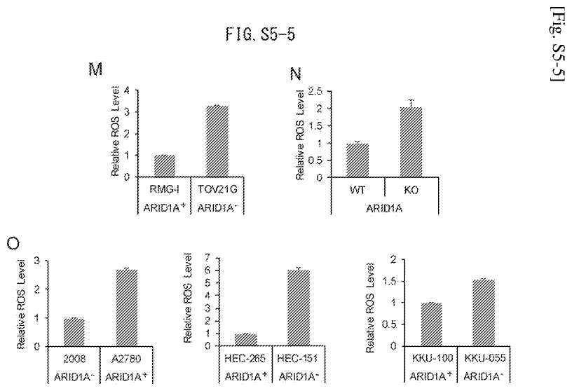

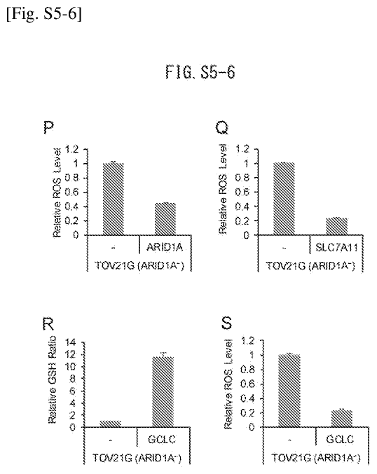

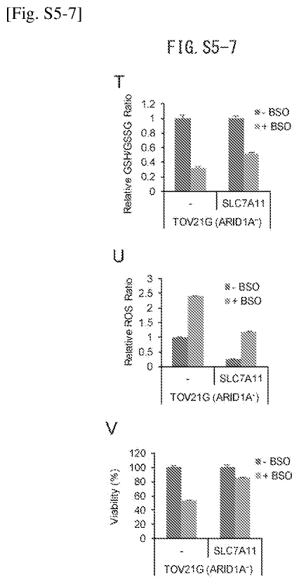

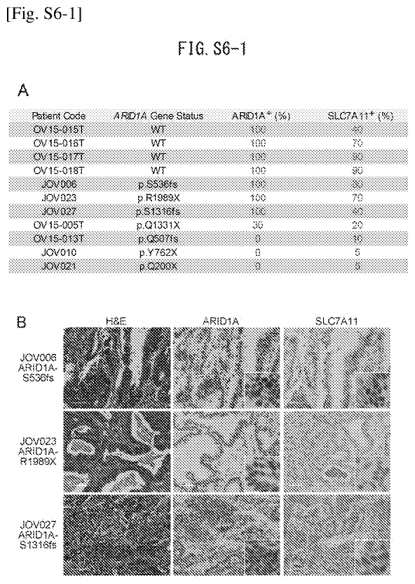

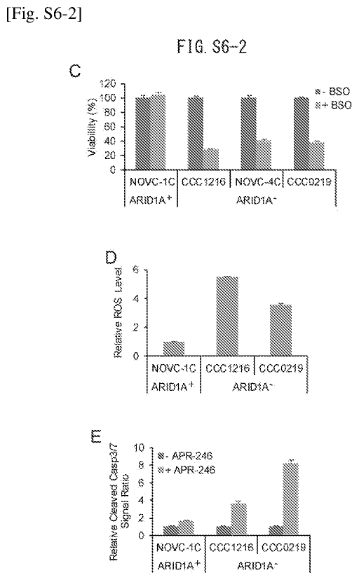

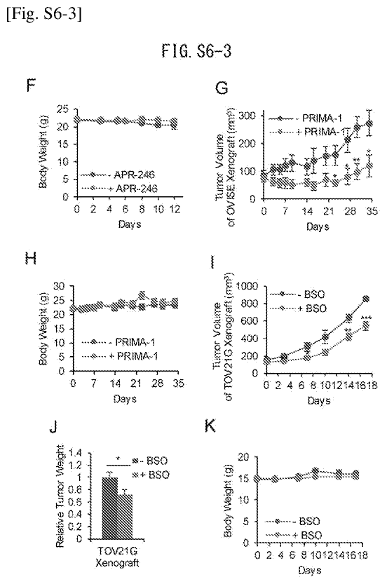

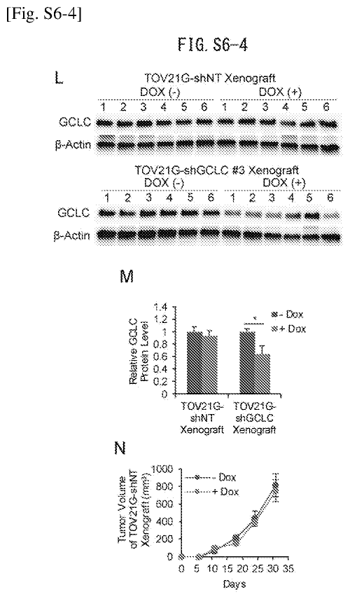

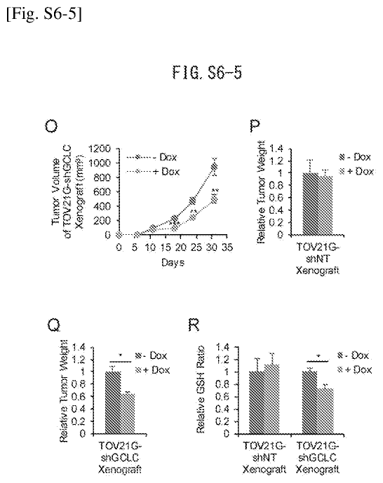

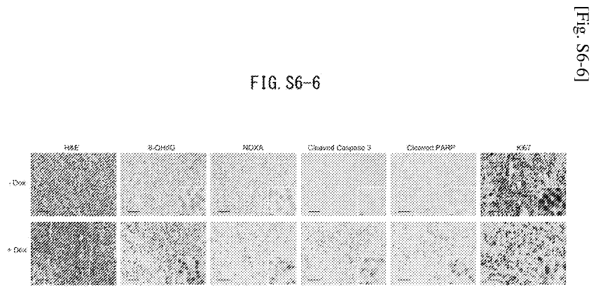

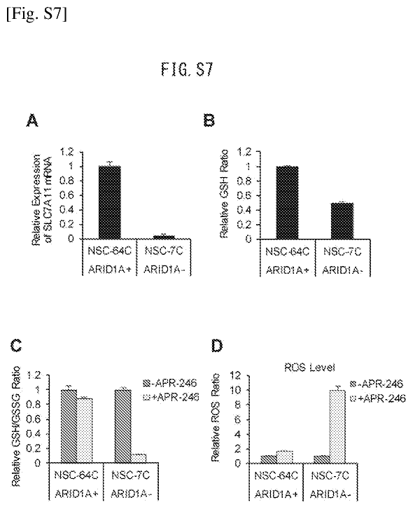

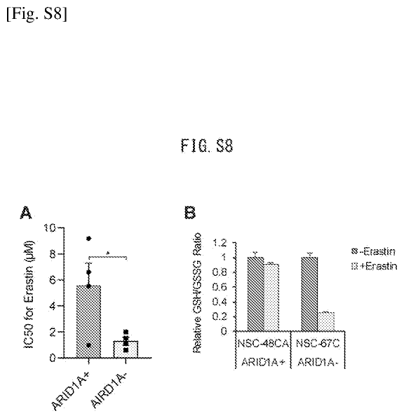

Similar to Example 4, the basal GSH level was lower in these SWI/SNF complexes-deficient cells than in these SWI/SNF complexes proficient cells (FIG. 20). APR-246 treatment decreased in the GSH level and increased in the ROS level of these SWI/SNF complexes-deficient cancer cells (FIGS. 21, 22). The decrease in the basal GSH level makes these SWI/SNF complexes-deficient cancer cells vulnerable to inhibition of the GSH metabolic pathway.

### Experimental Methods Regarding Examples 1-13

### Experimental Model and Subject

### (1) Establishment of Ovarian Cancer PDCs (Example 5)

Tumor samples and ascites were obtained from four ovarian cancer patients who underwent surgery or cell-free and concentrated ascites reinfusion therapy at the National Cancer Center Hospital or Kaname-cho Hospital (Tokyo, Japan) and were cultured in vitro. This protocol was approved by the Institutional Review Board of the National Cancer Center (Tokyo, Japan), and informed consent was obtained from the patients. To establish CCCO219 and CCC1216 PDCs, patient ascites (24 ml) were diluted in two volumes of PBS containing 2 mM EDTA, layered over 15 ml Ficoll-Paque PLUS (GE Healthcare), and centrifuged at 2200 rpm for 30 min. The interphase mononuclear layer was transferred to a fresh conical tube and washed twice with PBS containing 2 mM EDTA. Epithelial cells were labeled magnetically with microbeads conjugated to a monoclonal human epithelial antigen-125 antibody (EasySep Human EpCAM Positive Selection Kit, STEMCELL Technologies). Epithelial antigen-125-positive cells were collected by magnetic selection and cultured in DMEM/F-12 supplemented with 10% FBS. To establish NOVC-1C and NOVC-4C PDCs, whole ascetic cells were pelleted by centrifugation at 1500 rpm for 5 min at room temperature and then incubated in hemolysis buffer (0.75% NH4Cl and 17 mM Tris-HCl, pH 7.65) for 10 min. After centrifugation, pellets were washed with PBS and cultured in RPMI 1640 containing 10% FBS for 1 week. Thereafter, the culture medium was replaced with DMEM containing 10% FBS to remove lymphocytes, and cells were cultured for another week. Adherent cells were cultured in RPMI 1640 containing 10% FBS for several weeks, exchanging the medium once per week, until multiple colonies appeared. If needed, cultured cells were treated repeatedly with 0.05% trypsin-EDTA for a short duration to remove fibroblasts or other cell types such as mesothelial cells. The culture was passaged when colonies became dense. The ARID1A expression status was confirmed by immunoblot analysis.

### (2) Mouse Xenograft Model (Example 5)

All mouse experiments were approved by the National Cancer Center (NCC) animal Ethical Committee. Cells were counted and re-suspended in a 1:1 mixture of 100 μl culture medium and 100 μl Matrigel (BD Biosciences) on ice. Thereafter, cells (RMG-I: 2×10⁶ cells/mouse; TOV21G: 2×10⁵ cells/mouse for ARP-246 treatment and 1×10⁶ cells/mouse for BSO treatment; OVISE: 2×10⁶ cells/mouse; TOV21G-shNT: 2×10⁵ cells/mouse; and TOV21G-shGCLC: 2×10⁵ cells/mouse) were injected subcutaneously into the flank of 6-week-old female BALB/c-nu/nu mice (CLEA and Charles River). In the subcutaneous model, once the tumors were palpable (about 3-14 days after implantation), mice were randomly divided into two groups. In the drug treatment group, mice were injected intraperitoneally with either PBS or compounds [APR-246 (50 mg/kg), PRIMA-1 (25 mg/kg), or BSO (750 mg/kg)] once daily for 12-14 days. In the doxycycline (Dox) treatment study, TOV21G-shNT cells and TOV21G-shGCLC cells were injected into the flanks of 6-week-old female BALB/c-nu/nu mice. Once the tumors were palpable (13 days after implantation), mice were randomly divided into two groups and fed a diet containing Dox (625 ppm) or a control diet. In other experiments, TOV21G-shNT cells and TOV21G-shGCLC cells were treated with Dox (0.5 μg/ml) for 4 days and then injected into the flank of 6-week-old female BALB/c-nu/nu mice. The mice were then fed a diet containing Dox (625 ppm) or a control diet. Tumor growth was measured every several days using calipers. The volume of implanted tumors was calculated using the formula V=L×W2/2, where V is volume (mm³), L is the largest diameter (mm), and W is the smallest diameter (mm). At the end of the experiment, mice were sacrificed in accordance with standard protocols.

### (3) Cell Lines (Examples 1-13)

Cells were maintained in a humidified incubator containing 5% CO₂ at 37° C. in DMEM/F-12 (Wako) supplemented with 10% fetal bovine serum (FBS; Gibco/Life Technologies), 2 μmol/l glutamine, 100 U/ml penicillin, and 100 μg/mL streptomycin (Wako). TOV21G, HEC1A, H2228, H2122, H1819, H1299, H522, and H1703 cells were obtained from the American Type Culture Collection (ATCC). RMG-I, OVISE, HEC-265, HEC-151, KKU-100, KKU-055, Ishikawa, JHUEM-2, HuCCA-1, KP-4, JMU-RTK-2, G401, G402 and KMRC-1 cells were obtained from the Japanese Collection of Research Bioresources (JCRB) Cell Bank. JHUEM-2, SSP25, PC9, and HS-ES-1 cells were obtained from the Riken Cell Bank (RCB). 2008 and A2780 cells were provided by Drs. S. B. Howell and E. Reed. ARID1A-KO (Q456X/Q456X) and parental HCT116 cells were purchased from Horizon Discovery. HCC-44 cells were gifted from Gazdar, A. F. RCC-MF cells were obtained from Cell Lines Service. The cell lines were authenticated by verifying alterations of multiple cancer-related genes via sequencing. Cells were used for functional experiments after less than 3 months of passaging post-receipt. All cell lines tested negative for mycoplasma by MycoAlert (Lonza).

### (4) Generation of shRNA- and cDNA-Expressing Lentiviruses and Virus-Infected Cells (Example 3)

The shRNA-expressing lentiviral vectors pGIPZ (shNT, OHS4346; shp53, RHS4430-200289946) (Open Biosystems) and pTRIPZ (shNT, OHS5832; shGCLC #3, RHS4946_200777182), the cDNA-expressing lentiviral vectors (pLOC-GCLC, OHS5897_202616616; pLOC-SLC7A11, OHS5898_219582558; pLOC-NRF2, OHS5900-202624558) (all from ThermoFisher Scientific), (pLenti-puro-ARID1A, #39478) (Addgene), and packaging plasmids (psPAX2: #12260 and pMD2.G: #12259) (Addgene) were used for constitutive expression of shRNA or cDNAs. To generate virus, 293LTV cells were transfected with lentiviral plasmids and packaging plasmids using Lipofectamine 3000 (Invitrogen/ThermoFisher Scientific). On the following day, the medium was replaced with fresh growth medium and lentivirus-containing supernatants were harvested and concentrated by centrifugation. To establish cells infected with viral constructs, cells were transduced with lentiviral vectors and then incubated for 7-14 days in growth medium containing 2 μg/ml puromycin (Sigma-Aldrich) or 20 μg/ml blasticidin (Wako).

### (5) Establishment of Diffuse-Type Gastric Cancer Cell Lines (Example 6)

Tumor samples and ascites were obtained from patients with diffuse-type gastric cancer who underwent surgery or cell-free and concentrated ascites reinfusion therapy at the National Cancer Center Hospital or Kanamecho Hospital (Tokyo, Japan) and were cultured in vitro. The study protocol was approved by the Institutional Review Board of the National Cancer Center (Tokyo, Japan), and written informed consent was obtained from the patients. Whole ascetic cells were pelleted by centrifugation at 1500 rpm for 5 min at room temperature and then incubated in hemolysis buffer (0.75% NH₄Cl and 17 mM Tris-HCl, pH 7.65) for 10 min. After centrifugation, pellets were washed with PBS and cultured in RPMI 1640 containing 10% FBS for 1 week, after which the culture medium was replaced with DMEM containing 10% FBS to remove lymphocytes. Cells were cultured for an additional week. Adherent cells were cultured in RPMI 1640 containing 10% FBS for several weeks with weekly medium exchanges until the appearance of multiple colonies. When necessary, cultured cells were treated repeatedly with 0.05% trypsin-EDTA for a short duration to remove fibroblasts or other cell types such as mesothelial cells. The culture was passaged when colonies became dense.

Method

### (1) Drug Library Screen (Example 1)

ARID1A-WT and ARID1A-KO HCT116 cancer cells were used for screening assays. Cells were seeded in 96-well plates, incubated for 24 hrs, and then treated with the drug at a concentration of 0.1, 1, or 10 μM [SCADS Inhibitor Kit, including 334 compounds (Table S1)]. Cell viability was assessed after 5 days using the CellTiter-Glo Luminescent Cell Viability Assay (Promega). Luminescence was measured using an Envision Multi-label plate reader (PerkinElmer). The luminescence reading was used to determine the cell viability relative to that of cells treated with solvent (DMSO). Candidate compounds were considered if viability of ARID1A-KO cells was less than 40% that of ARID1A-WT cells was more than 80%.

### (2) Cell Viability Assay (Examples 1-5, 10-12)

Cell viability was examined by measuring the cellular ATP level using the CellTiter-Glo Luminescent Cell Viability Assay (Promega). To measure cell viability after siRNA-mediated knockdown, cell lines were transfected with siRNAs (25 nM) using Lipofectamine RNAiMAX. After 48 hrs, cells were trypsinized and repeatedly transfected with siRNAs (25 nM) using Lipofectamine RNAiMAX. Cells were trypsinized after a further 48 hrs, counted, and reseeded at the specified density in 96-well plates. To measure cell viability after drug treatment, cells were trypsinized, counted, reseeded at the specified density in 96-well plates, and exposed to the indicated concentrations of drugs. Cell viability was measured using the CellTiter-Glo Luminescent Cell Viability Assay. Luminescence was measured using an Envision Multi-label plate reader (PerkinElmer).

### (3) Colony Formation Assay (Examples 1, 3)

The effect of drug treatment on cancer cell survival was evaluated in a colony formation assay. Cells were trypsinized, counted, reseeded at the specified density in 12-well plates, exposed to the indicated concentrations of drugs for 10-14 days, and fixed for 10 min in 50% (v/v) methanol containing 0.01% (w/v) crystal violet. Images were taken on LAS-3000 Imaging System (Fujifilm) and colonies were counted using Multi Gauge software.

### (4) Annexin V/Propidium Iodide (PI) Staining Assay (Examples 1, 3)

The Annexin V-FITC/PI Apoptosis Detection Kit (Sigma-Aldrich) was used to detect apoptotic cells. Briefly, the cell pellet was suspended in 1× binding buffer and then incubated with Annexin V-FITC and PI in the dark for 10 min. Fluorescence was analyzed on a Guava flow cytometer (Millipore). Data were analyzed using GuavaSoft software (v. 2.7). Relative ratios of the Annexin V-positive fraction in treated samples were normalized against untreated samples.

### (5) Detection of, GSH, ROS and Cleaved Caspase-3/7 in Cell Lines (Examples 1-5, 13)

GSH, ROS and apoptosis were detected using the GSH/GSSG-Glo Assay (Promega) and/or the GSH-Glo Assay (Promega), the ROS-Glo Assay (Promega), and the Caspase-Glo 3/7 Assay, respectively. To measure levels of GSH, ROS and apoptosis after drug treatment, cells were trypsinized, counted, reseeded at the specified density in 96-well plates and exposed to the indicated concentrations of drugs. After 16-48 hrs, luminescence was measured using an Envision Multi-label plate reader (PerkinElmer). To measure levels of GSH and ROS after siRNA-mediated knockdown, cell lines were transfected with siRNAs (25 nM) using Lipofectamine RNAiMAX. After 48 hrs, cells were trypsinized and transfected repeatedly with siRNAs (25 nM) using Lipofectamine RNAiMAX. Cells were trypsinized after a further 48 hrs, counted, and reseeded at the specified density in 96-well plates. After 72-120 hrs, luminescence was measured using an Envision Multi-label plate reader (PerkinElmer). Cell viability was also measured using the CellTiter-Glo Luminescent Cell Viability Assay (Promega). Caspase-3/7, GSH, and ROS levels were normalized against cell viability. The GSH/GSSG ratio was calculated as the GSH-GSSG signal divided by the GSSG/2 signal. Relative signal ratios in treated samples were normalized against untreated samples.

### (6) Detection of GSH in Tumor Samples (Examples 5, 13)

GSH was detected using the GSH-Glo Assay (Promega). Tumor samples derived from xenografts were weighed and washed with PBS. The tumor samples were mixed with 50 μl of PBS and homogenized using a Mini Cordless Grinder (Funakoshi). PBS (950 μl) was added to homogenized tumor samples and centrifuged at 4° C. for 10 min at 15,000 rpm. Tumor extract and 2×GSH-Glo Reagent (25 μl of each) were mixed in white 96-well plates (Greiner) and incubated for 30 min at room temperature. Luciferin Detection Reagent (50 μl) was added and the samples were incubated for 15 min at room temperature. Luminescence was measured using an Envision Multi-label plate reader (PerkinElmer). GSH signal intensities per 1 mg of tumor sample were calculated. Relative GSH ratios were normalized against untreated samples (without APR-246 or Dox).

### (7) Detection of TrxR Activity (Example 2)

TrxR activity including TrxR1, TrxR2 and TrxR3 was measured using the Thioredoxin Reductase Assay Kit (Abcam). Cells were trypsinized, counted, reseeded at the specified density in 10 cm dishes and exposed to the indicated concentrations of APR-246. After 24 hrs, cells were washed with cold PBS and lysed with buffer containing a proteinase inhibitor. After centrifugation, the supernatant was supplemented with a TrxR inhibitor and incubated for 20 min at 25° C. Absorbance was measured using an Envision Multi-label plate reader (PerkinElmer). Relative TrxR ratios were normalized against untreated samples.

### (8) Cell Stress Profiling by Antibody Array (Example 2)

Antibody array analysis was conducted using the Human Cell Stress Array (R&D Systems). For whole-cell extraction, 1×10⁷ cells were harvested, washed with PBS, lysed in Lysis Buffer 6 supplemented with a proteinase inhibitor cocktail and a phosphatase inhibitor cocktail (Active Motif), incubated for 30 min on ice, and centrifuged at 4° C. for 10 min at 15,000 rpm. Whole-cell lysates (1 ml) were mixed with 0.5 ml of Array Buffer 4 and 20 μl of reconstituted Detection Antibody Cocktail for 1 hr at room temperature. These samples were added to membranes blocked with Array Buffer 4. After incubation overnight at 4° C., the membranes were washed twice with 1× Wash Buffer and rinsed with distilled water and then dried. Diluted streptavidin-HRP (2 ml) was added and the membrane was incubated for 30 min at room temperature and then was washed with 1× Wash Buffer. Chemi Reagent Mix was applied evenly to the membrane and incubated for 1 min. Chemiluminescence signals were measured using LAS-3000 Imaging System (Fujifilm). Signal intensities were measured using Multi Gauge software. The ratios of signal intensities in cells treated with 40 μM APR-246 for 24 hrs were calculated relative to the corresponding intensities in untreated cells.

### (9) Transcriptomic Profiling by Microarray (Examples 1, 4)

Total RNA was extracted using the Qiagen RNeasy kit. The integrity of extracted RNA was confirmed by NanoDrop spectrophotometry (NanoDrop Technologies). Total RNA was reverse-transcribed using the Agilent Low Input Quick Amp Labeling Kit (Agilent Technologies). cDNA was hybridized for 16 hrs at 65° C. on duplicate Agilent microarrays (SurePrint G3 Human Gene Expression 8×60K Ver. 1.0, G4851: 42405 probes) using the Gene Expression Hybridization Kit (Agilent Technologies). After the arrays were washed using the Gene Expression Wash Pack (Agilent Technologies), data were extracted using an Agilent scanner. The arrays were analyzed initially using Feature Extraction software (Agilent Technologies). A quantitative signal and qualitative detection call were generated for each sample and transcript.

Data files were subsequently analyzed, normalized, and compared using GeneSpring GX12.6 (Agilent Technologies). Raw expression data of 42,545 probe sets on SurePrint G3 Human Gene Expression arrays were processed and log 2-transformed. Expression data for each sample were normalized against median expression levels in the control condition. Genes were grouped according to fold changes. All raw microarray data files have been deposited in the Gene Expression Omnibus (GEO: GSE122925 and GSE122926).

### (10) Quantitation of mRNA (Examples 1-5, 13)

mRNA was extracted and cDNA was synthesized using the SuperPrep (Registered Trademark) Cell Lysis & RT Kit for qPCR (TOYOBO). Aliquots of cDNA were subjected to quantitative PCR using the SuperPrep/THUNDERBIRD Probe qPCR Set (TOYOBO) and TaqMan Gene Expression Assays (Life Technologies). The following gene-specific primer/probe sets were used: NOXA (PMAIP1) (Hs00560402 ml), NRF2 (NFE2L2) (Hs00975961 g_1), GCLC (Hs00155249 ml), GSS (Hs00609286_m1), and SLC7A11 (Hs00921938_m1). PCR was performed in an ABI StepOnePlus Real-Time PCR System (Life Technologies) under the following conditions: denaturation at 95° C. for 15 s, followed by annealing and extension at 60° C. for 30 s (40 cycles). For each sample, the mRNA levels of target genes were normalized against levels of GAPDH mRNA. The target/GAPDH ratios were then normalized against those in control samples using the 2-ΔΔCt method.

### (11) Immunoblot Analysis (Examples 1-5, 12)

For whole-cell extraction, 5×10⁵ cells were harvested, washed with PBS, lysed in NETN420 buffer [20 mM Tris-HCl (pH 7.5), 420 mM NaCl, 0.5% NP-40, and 1 mM EDTA] supplemented with a proteinase inhibitor cocktail and a phosphatase inhibitor cocktail (Active Motif), incubated for 30 min on ice, and centrifuged at 4° C. for 10 min at 15,000 rpm. The soluble fractions of whole-cell lysates were mixed with SDS sample buffer. For cell extraction including the membrane fraction to detect SLC7A11, cells were harvested, washed with PBS, lysed in M-PER Mammalian Protein Extraction Regent Buffer (ThermoFisher Scientific) supplemented with a proteinase inhibitor cocktail and a phosphatase inhibitor cocktail (Active Motif), incubated for 10 min on ice, and centrifuged at 4° C. for 10 min at 15,000 rpm. The soluble fractions were mixed with SDS sample buffer. Tumor samples derived from xenografts were weighed and washed with PBS. The tumor samples (10 mg) were mixed with 50 μl of NETN420 buffer supplemented with a proteinase inhibitor cocktail and a phosphatase inhibitor cocktail (Active Motif) and homogenized using a Mini Cordless Grinder (Funakoshi). The homogenized tumor samples were diluted in an additional 450 μl of NETN420 buffer, incubated for 30 min on ice, and centrifuged at 4° C. for 10 min at 15,000 rpm. The soluble fractions of whole-cell lysates were mixed with SDS sample buffer. Proteins were separated by SDS-PAGE, transferred to PVDF membranes, and immunoblotted with the indicated antibodies. β-actin was used as a loading control. Membranes were blocked overnight at 4° C. or for 1 hr at 25° C. with PVDF Blocking Reagent for Can Get Signal (TOYOBO) and then probed with Can Get Signal Solution 1 (TOYOBO) containing primary antibodies. After washing with TBS containing 0.1% Tween 20, the membranes were incubated with TBS containing 0.1% Tween 20, 1% BSA, and horseradish peroxidase-conjugated anti-mouse or anti-rabbit secondary antibodies, and visualized using Western Lightning ECL Pro (Perkin Elmer). Chemiluminescence signals were measured using LAS-3000 Imaging System (Fujifilm). Signal intensities were measured using Multi Gauge software. The protein levels of GCLC were normalized against the levels of (β-actin. The GCLC/(β-actin ratios were then normalized against those in control samples without Dox treatment. Data are expressed as the mean±SEM (n=6). The following antibodies were used for immunoblotting: GCLC (Abcam, ab190685), NRF2 (Abcam, ab62352), p53 (Calbiochem, OP43), β-actin (CST, 4790), ARID1A (CST, 12354), BRG1 (CST, 49360), SLC7A11 (CST, 12691), NOXA (CST, 14766), p21 (CST, 2947), JNK (Santa Cruz Biotechnology, sc-7345), and phospho-JNK (Santa Cruz Biotechnology, sc-6254).

### (12) ChIP Assay (Example 4)

1×10⁶ cells were harvested 24 hrs after seeding and treated with 1% formaldehyde for 10 min at room temperature to crosslink proteins to DNA. Glycine (0.125 M) was added to stop the crosslinking process. ChIP assays were performed using the ChIP-IT Express Enzymatic kit (Active Motif) and antibodies against ARID1A (CST, 12354), BRG1 (CST, 49360), NRF2 (CST, 12721) or RNAPII (Active Motif, 39097). Purified DNA was subjected to quantitative PCR using the SuperPrep/THUNDERBIRD SYBR qPCR Set (TOYOBO) and the following primer pairs: SLC7A11-1182-1099-F (5′-TCAGAAGCTTATTTAATGGTGCG-3′) and SLC7A11-1182-1099-R (5′-GTGGTTTTGGATTCAGTGAGAAG-3′); SLC7A11-297-241-F (5′-CAGCTTTTGTTGCTCACTACG-3′) and SLC7A11-297-241-R (5′-TCGGAACAGACCTTCCCAG-3′); SLC7A11-14_79-F (5′-GAGGAAGCTGAGCTGGTTTG-3′) and SLC7A11-14_79-R (5′-GCATCGTGCTCTCAATTCTC-3′); SLC7A11_71_190-F (5′-GCACGATGCATACACAGGTG-3′) and SLC7A11_71_190-R (5′-CCTCTGCTTTCAGACTGTCT-3′); and SLC7A11_972_1070-F (5′-CGGAGTGTTCAGCAGAAGTC-3′) and SLC7A11_972_1070-R (5′-GAGGTGACAAGCACATGAAC-3′). The PCR conditions were as follows: denaturation at 95° C. for 15 s, followed by annealing and extension at 60° C. for 60 s (45 cycles). PCR was performed on an ABI StepOnePlus Real-Time PCR System (Life Technologies). Protein enrichment was expressed as a percentage of input.

### (13) Metabolome Analysis (Example 4)

Metabolites were extracted from 2×10⁶ cells. Culture medium was removed, and cells were washed twice with 5% mannitol solution (8 ml and then 4 ml) and then treated with 800 μl methanol and 150 μl Milli-Q water containing 5 μg 2-isopropylmalic acid as an internal control. The metabolite extract was transferred to a microfuge tube and dried using a Spin Dryer (TAITEC). Derivatization in the solid phase was conducted as described below. The solid phase cartridge Presh-SPE AOS was supplied by AiSTI SCIENCE (Wakayama). Cell extract was mixed with 200 μl Milli-Q water and 800 μl acetonitrile and incubated at 37° C. for 30 min. After centrifugation at 14,000 rpm for 5 min at 4° C. the supernatant was transferred to a new tube. The derivatization conditions were 3 min of methoximation with 5 μl of >5% methoxyamine solution and 10 min of trimethylsilylation with 25 μl N-methyl-N-trimethylsilyl-trifluoroacetamide. Derivatized analytes were effectively eluted with 100 μl n-hexane, and 1.0 μl of the derivatized solution was injected into the gas chromatograph/mass spectrometer GCMS-TQ8050 (Shimadzu).

Metabolome analysis was performed on a GCMS-TQ8050 equipped with a BPX-5 capillary column (internal diameter: 30 m×0.25 mm; film thickness: 0.25 μm; SEG, Victoria). Parameter setting was described previously (See, Nishiumi et al., Oncotarget 8, 17115-17126, 2017). During GCMS-TQ8050 analysis, the inlet temperature was kept at 250° C. and helium was used as a carrier gas at a constant flow rate of 39.0 cm per sec. The injector split ratio was set to 1:10. The GC column temperature was programmed to remain at 60° C. for 2 min and then to rise from 60° C. to 330° C. at a rate of 15° C. per min, before being kept at 330° C. for 3 min. The total GC run time was 23 min. The transfer-line and ion-source temperatures were 280° C. and 200° C., respectively. The ionization voltage was 70 eV. Argon gas was used as a collision-induced dissociation gas. Metabolites were detected using the Smart Metabolites Database (Shimadzu), which contains the relevant MRM method file and data regarding the GC analytical conditions, MRM parameters, and retention index employed for metabolite measurements. The Automatic Adjustment of Retention Time (AART) function of GCMS solution software (Shimadzu) and a standard alkane series mixture (C7 to C33) were used to correct the retention time. Peaks were identified automatically and confirmed manually based on the specific precursor and product ions and the retention time. Relative cysteine ratios were normalized against ARID1A-proficient cells.

### (14) Targeted and Whole-Exome Sequencing (Example 5)

Targeted sequencing was conducted using 1.0 μg DNA extracted from cultured cancer cells. Targeted genome capture was performed using the Agilent SureSelect kit NCC Oncopanel (931196). Sequencing was performed on the Illumina NextSeq platform using 150 bp paired-end reads (Illumina). Basic alignment and sequence quality control were conducted using the Picard and Firehose pipelines. Reads were aligned against the reference human genome from the UCSC human genome 19 (hg19) using the Burrows-Wheeler Aligner Multi-Vision software package. Duplicate reads were generated during PCR; therefore, paired-end reads that aligned to the same genomic positions were removed using SAMtools. Somatic single-nucleotide variants were called by the MuTect program, which applies a Bayesian classifier to allow detection of somatic mutations with low allele frequencies. Somatic insertion/deletion mutations (indels) were called using the GATK Somatic IndelDetector (http://archive.broadinstitute.org/cancer/cga/indelocator).

### (15) Immunohistochemistry (Example 5)

Eleven patients were diagnosed with ovarian cancer and underwent surgery at the National Cancer Center Hospital (NCCH), Tokyo, Japan, or at the Jikei University Hospital (JUH), Tokyo, Japan. None of the 11 patients had received any pre-surgical treatment. This study was approved by the Institutional Review Board of the National Cancer Center (Tokyo, Japan) and Jikei University, and informed consent was obtained from the patients. Ovarian tumors were diagnosed in accordance with the International Federation of Gynecology and Obstetrics (FIGO) guidelines and classified according to the World Health Organization (WHO) classification system. ARID1A mutations were determined by target and whole-exome sequencing, as described previously (See, Kanke et al., Oncotarget 9, 6228-6237, 2018).

Formalin-fixed, paraffin-embedded ovarian cancer clinical specimens and TOV21G xenografts were deparaffinized and representative whole 4-μm-thick sections were analyzed by IHC. TOV21G-shGCLC xenografts were embedded in OTC compound (25608-930; Tissue-Tek) and stored at −80° C. The samples were removed from the freezer and equilibrated at −20° C. for approximately 15 minutes before sectioning. Tissue sections (6 μm thick) were placed on positively charged slides, dried, and fixed for 15 minutes at room temperature in 3% formaldehyde, followed by 5 minutes in methanol at −20° C. After fixation, representative sections were analyzed by IHC. Tissue sections were stained using antibodies against Ki-67 (MIB-1) (GA62661-2, 1:100 dilution; Dako), cleaved caspase-3 (5A1E) (9664, 1:200 dilution; CST), cleaved PARP (D64E10) (5625, 1:100 dilution; CST), NOXA (114C307) (ab13654, 1:2000 dilution; Abcam), 8-hydroxy-2′-deoxyguanosine (N45.1) (ab48508, 1:500 dilution; Abcam), ARID1A (HPA005456, 1:2000 dilution; Sigma-Aldrich), and anti-SLC7A11 (xCT) (ab175186, 1:400 dilution; Abcam). All IHC staining was performed using a Dako autostainer Link48 (Dako).

Immunohistochemical staining for 8-OHdG was further evaluated by a semiquantitative approach used to assign a histological score (H-score) to tumor samples (See, Hirsch et al., J Clin Oncol 21, 3798-3807, 2003). First, membrane staining intensity (0, 1+, 2+, or 3+) was determined for each cell in a fixed field. The H-score was assigned using the following formula: [1×(% cells 1+)+2×(% cells 2+)+3×(% cells 3+)]. The final score, ranging from 0 to 300, gives more relative weight to higher-intensity membrane staining in a given tumor sample. The percentage of NOXA-, cleaved caspase-3-, cleaved PARP- and Ki67-positive cells (of the total number of cells) in each slide were counted.

### (16) Whole Exome Sequencing (Example 6)

Exome libraries were generated using the Agilent SureSelect XT (Agilent, Palo Alto, Calif.) according to the manufacturer's protocol. Prior to sequencing, exome libraries were analyzed using the Agilent BioAnalyzer 2200 with the High Sensitivity D1000 (Agilent) and QuantStudio bFlex (Thermo Fisher Scientific, Rockford, Ill.) with KAPA Library Quantification Kit (IIlumina, San Diego, Calif.). All libraries were sequenced using the Illumina HiSeq 2000/2500 system with TruSeq SBS Kit v3-HS (200-cycles) reagents (Illumina). Sequence data were mapped to the Genome Reference Consortium GRCh37 assembly using BWA-MEM. Somatic variant calling was conducted using GATK4 Mutect2.

### (17) Histological Analysis of Cell Line-Derived Xenografts (Example 6)

Six-week-old female CAnN.Cg-Foxn1nu/CrlCrlj (BALB/c-nu/nu) mice (Charles River Laboratories Japan were bred at room temperature with a 12 h light/dark daily cycle. The mice were maintained under specific pathogen-free conditions and were provided sterile food, water, and cages. Approximately 5×10⁶ cancer cells were suspended with 100 μl phosphate-buffered saline and were injected subcutaneously into mice using a 26.5-gauge needle. All experiments were conducted in accordance with the ethical guidelines of the International Association for the Study of Pain and were approved by the Committee for Ethics in Animal Experimentation of the National Cancer Center. Specimens fixed in formalin and embedded in paraffin were cut into 8 μm sections, which were dewaxed and dehydrated for routine hematoxylin and eosin staining.

Quantification and Statistical Analysis

### (1) Statistical Analysis (Examples 1 to 5)

Statistical analyses were performed using Microsoft Excel. Data are expressed as the mean±SD or mean±SEM, as indicated in the figure legends. The sample size (n) is indicated in the figure legends and represents biological replicates. Statistical significance was evaluated using the two-tailed Student's t-test. Statistically significant differences are indicated by asterisks as follows: *p<0.05, **p<0.01, and ***p<0.001.

TABLE S1-1
Cell Viabilities of HCT116 ARID1A-WT and
ARID1A-KO in treatment with 334 Compounds
at 10 μM for 5 days. Related to FIGS. 1-1 to 1-7.

| Drug | WT | KO |
|---|---|---|
| (+)-JQ1 | 6.310463 | 10.73684 |
| 1400 W, HCl | 184.5452 | 132.9235 |
| 17-AAG | 8.063274 | 8.213512 |
| 1-Azakenpaullone | 95.89057 | 114.0704 |
| 2′,5′-dideoxyadenosine | 197.8482 | 172.2073 |
| 3-ATA | 155.5141 | 86.76754 |
| 4-cyano-3-methylisoquinoline | 142.6645 | 163.7513 |
| 5,15-DPP | 90.1195 | 98.72596 |
| 5-FU | 32.167 | 24.06869 |
| A23187 | 6.25447 | 8.66759 |
| A83-01 | 77.91767 | 104.8454 |
| a-Amanitin | 8.977325 | 10.20816 |
| ABT-702 | 94.10939 | 111.7532 |
| ABT-737 | 94.37021 | 89.51014 |
| ABT-888 (Veliparib) | 62.68696 | 53.34906 |
| Aclarubicin | 4.479395 | 5.527251 |
| Actinomycin D | 6.951308 | 6.530922 |
| Actinonin | 88.53803 | 103.1408 |
| AG014699 (Rucaparib) | 18.73871 | 15.97436 |
| AG1024 | 161.1636 | 183.5477 |
| AG1296 | 190.9406 | 144.7899 |
| AG1478 | 149.2606 | 129.9442 |
| AG490 | 152.9882 | 114.4173 |
| AG825 | 85.83 | 80.85553 |
| AG957 | 23.69447 | 17.53071 |
| AGL 2263 | 148.6873 | 172.9001 |

TABLE S1-2

| Drug | WT | KO |
|---|---|---|
| AKT inhibitor | 212.0073 | 143.282 |
| Akt Inhibitor IV | 3.275849 | 5.522605 |
| Akt Inhibitor VIII, Isozyme- | 84.50246 | 124.843 |
| Selective, Akti-½ | | |
| Akt Inhibitor XI | 119.2398 | 137.7911 |
| ALLN | 33.808 | 39.85302 |
| Alsterpaullone, 2-cyanoethyl | 6.819688 | 14.54572 |
| Amastatin | 101.9296 | 109.6659 |
| AMD3100 octahydrochloride | 102.6908 | 102.0251 |
| AMI-1 | 88.42053 | 85.52387 |
| Amiloride | 105.7076 | 113.3092 |
| Aminoglutethimide | 107.7597 | 107.2134 |
| Aminoguanidine, HCl | 187.7374 | 128.2305 |
| AMT, HCl | 171.2223 | 139.818 |
| Anacardic acid | 102.71 | 112.6464 |
| anisomycin | 5.36335 | 7.32698 |
| Antimycin A1 | 4.328278 | 7.110865 |
| Aphidicolin | 14.02206 | 11.91168 |
| ATM kinase inhibitor | 70.42186 | 83.20541 |
| ATM/ATR kinase inhibitor | 59.07151 | 76.76988 |
| Aurora kinase inhibitor II | 53.336 | 60.46446 |
| Aurora kinase inhibitor III | 133.1405 | 172.0424 |
| Aurora kinase/cdk inhibitor | 5.038885 | 11.31124 |
| Axitinib | 36.39137 | 42.56131 |
| AY 9944 | 31.41395 | 50.15009 |
| Azacytidine | 86.53938 | 43.39377 |
| AZT | 71.38478 | 60.26997 |
| BADGE | 93.58725 | 107.888 |

TABLE S1-3

| Drug | WT | KO |
|---|---|---|
| Bafilomycin AI | 4.059043 | 4.345287 |
| Baicalein | 94.60898 | 93.67433 |
| Benzamide | 188.0788 | 133.8889 |
| Benzylguanine | 88.83592 | 105.1044 |
| Bestatin | 68.58906 | 66.16346 |
| BH3I-1 | 99.90798 | 109.7951 |
| BIO | 87.18754 | 107.4068 |
| Bisindolymaleimide I, HCl | 8.076613 | 6.105568 |
| BIX01294 | 2.56518 | 4.494297 |
| Bleomycin sulfate | 27.28077 | 30.20309 |
| BMS-345541 | 11.89522 | 19.70272 |
| bortezomib | 3.062321 | 5.806336 |
| BPIQ-II | 119.0514 | 163.4598 |
| brefeldin A | 1.838193 | 4.57789 |
| b-Rubromycin | 88.70236 | 93.87001 |
| BSI-201 (Iniparib) | 63.46541 | 93.27831 |
| C646 | 93.09491 | 115.173 |
| C75 | 103.4877 | 93.63457 |
| CA-074 | 107.6022 | 115.0436 |
| Camptothecin | 2.965447 | 4.028216 |
| Cantharidin | 26.24581 | 18.18492 |
| CCG-1423 | 84.86764 | 99.56037 |
| Cdk½ inhibitor III | 8.590127 | 18.11673 |
| Cdk 2/9 inhibitor | 6.308507 | 14.25643 |
| Cdk4 inhibitor | 88.44717 | 96.22038 |
| Cerulenin | 69.96997 | 68.64001 |
| cFMS Receptor | 105.362 | 152.1544 |
| Tyrosine Kinase Inhibitor | | |

TABLE S1-4

| Drug | WT | KO |
|---|---|---|
| Chetomin | 4.207858 | 4.691916 |
| Chk2 inhibitor | 61.5345 | 86.38538 |
| Chk2 inhibitor II | 104.0524 | 139.2543 |
| chlorpromazine hydrochloride | 7.830475 | 15.56399 |
| Cisplatin | 129.6545 | 119.7587 |
| Clofibrate | 109.6044 | 111.2456 |
| compound C | 6.446944 | 9.380724 |
| cPLA2inhibitor | 125.7674 | 108.3777 |
| crizotinib | 2.293025 | 5.88423 |
| CT99021 | 63.83734 | 73.46473 |
| Cucurbitacin I | 13.95294 | 15.24509 |
| Cycloheximide | 9.325605 | 10.1479 |
| cyclopamine | 49.29074 | 86.68658 |
| Cyclosporin A | 29.89396 | 37.89064 |
| Cytochalasin D | 63.00134 | 59.86316 |
| Cytostatin | 10.14351 | 7.492823 |
| D4476 | 93.40018 | 124.271 |
| D609 | 181.173 | 133.5965 |
| Damnacanthal | 184.926 | 138.2154 |
| DAPT | 93.11721 | 103.8309 |
| dasatinib | 33.47713 | 37.82269 |
| Daunorubicin, HCl | 5.209388 | 4.454747 |
| Debromohymenialdisine (DBH) | 62.94855 | 72.17898 |
| Decitabine | 30.82561 | 30.35854 |
| Decylubiquinone | 85.68976 | 100.1951 |
| Deoxynojirimycin | 106.0549 | 120.9276 |
| Dephostatin | 90.35245 | 60.23909 |

TABLE S1-5

| Drug | WT | KO |
|---|---|---|
| Deprenyl | 104.0672 | 122.1482 |
| Dequalinium | 6.379096 | 5.244783 |
| desipramine hydrochloride | 34.62779 | 63.14738 |
| Dexamethasone | 211.7357 | 161.8217 |
| DFMO | 98.80422 | 117.8191 |
| Diacylglycerol kinase inhibitor II | 140.8741 | 171.4079 |
| Diazoxide | 102.6821 | 115.302 |
| DIDS | 97.03657 | 107.2053 |
| Diltiazem | 102.3198 | 114.8026 |
| Dimethyloxalylglycine | 95.769 | 119.9436 |
| Dioctanoylglycol | 99.78282 | 109.4112 |
| DMAT | 113.7397 | 116.7866 |
| Doxorubicin, HCl | 4.808619 | 4.139408 |
| E-64d | 100.8821 | 100.5678 |
| Ellagic acid | 127.2288 | 160.5371 |
| ENMD-2076 | 9.157804 | 10.05441 |
| ERK inhibitor II | 103.8555 | 160.6702 |
| erlotinib | 48.0992 | 48.60741 |
| Etoposide (VP-16) | 30.61667 | 27.37681 |
| ETYA | 95.86103 | 110.2846 |
| everolimus | 77.4291 | 77.11739 |
| FH535 | 92.74757 | 100.5931 |
| Finasteride | 99.64321 | 107.5569 |
| FK-506 | 173.1818 | 130.4366 |
| Flt-3 Inhibitor | 141.9634 | 177.725 |
| Flutamide | 162.8601 | 126.2496 |
| Formestane | 107.6191 | 69.45005 |

TABLE S1-6

| Drug | WT | KO |
|---|---|---|
| FTI-276 | 151.7653 | 118.2926 |
| Fumagillin | 96.42703 | 82.15807 |
| Fumitremorgin C | 90.40506 | 97.38972 |
| Fumonisin B1 | 87.22919 | 83.32826 |
| Gant61 | 13.28245 | 10.74406 |
| gefitinib | 54.46255 | 72.36471 |
| Genistein | 173.1879 | 117.6696 |
| GGTI-286 | 133.2565 | 123.5827 |
| Glibenclamide | 108.2879 | 109.3963 |
| GM 6001 | 187.6768 | 123.3845 |
| Go7874 | 3.010079 | 4.255706 |
| GSK-3 inhibitor II | 199.3658 | 164.3879 |
| GSK-3 inhibitor IX | 105.4779 | 147.7846 |
| H-1152 | 40.17893 | 49.18956 |
| H-7 | 116.4721 | 70.52231 |
| H-89 | 78.80581 | 119.4541 |
| HA 14-1 | 114.2556 | 119.1814 |
| HA1077 | 154.9647 | 73.79689 |
| HR22C16 | 12.51683 | 17.38612 |
| Hydroxyurea | 157.0498 | 116.7671 |
| IBMX | 159.7152 | 114.2526 |
| IC60211 | 131.0236 | 174.6736 |
| IKK-2 inhibitor VI | 19.28309 | 22.26123 |
| imatinib mesylate | 37.18296 | 55.37165 |
| indirubin-3′-monoxime | 105.9954 | 124.9767 |
| Ionomycin | 31.82186 | 35.8904 |
| IRAK-¼ inhibitor | 142.234 | 160.7174 |

TABLE S1-7

| Drug | WT | KO |
|---|---|---|
| isogranulatimide | 118.126 | 151.2771 |
| IWP-2 | 78.56061 | 90.80356 |
| IWR-1-endo | 76.33962 | 75.98279 |
| JAR Inhibitor I | 114.1598 | 148.9337 |
| JAK3 Inhibitor VI | 7.508911 | 17.2416 |
| Jervine | 74.38193 | 90.14013 |
| JNK inhibitor VIII | 127.134 | 189.4425 |
| Kenpaullone | 66.17778 | 35.00612 |
| KN-62 | 113.3229 | 131.0212 |
| KN93 | 20.88666 | 23.46684 |
| KT 5823 | 102.1181 | 111.294 |
| Lactacystin | 32.63204 | 26.05485 |
| lapatinib | 52.69583 | 78.35191 |
| Lavendustin C | 109.43 | 142.7562 |
| LDN193189 | 2.831618 | 2.712233 |
| lenalidomide | 91.13951 | 111.8631 |
| Leptomycin B* | 5.031341 | 4.968971 |
| LFM-A13 | 93.87647 | 106.7176 |
| Lidocaine | 102.8814 | 110.5194 |
| L-NMMA | 167.9828 | 116.4094 |
| Lonidamine | 98.38485 | 116.0356 |
| Lovastatin | 132.3259 | 85.17967 |
| LY 83583 | 4.357463 | 4.787581 |
| LY2157299 | 91.30732 | 113.5072 |
| LY294002 | 127.2893 | 119.3969 |
| Manumycin A | 61.21698 | 52.57154 |
| MDM2 inhibitor | 100.4322 | 116.4344 |

TABLE S1-8

| Drug | WT | KO |
|---|---|---|
| MDV3100 | 88.83505 | 94.90915 |
| MEK inhibitor I | 77.60282 | 117.4589 |
| Methotrexate | 42.96229 | 43.86443 |
| MG-132 | 3.448067 | 2.450934 |
| Mifepristone | 95.86234 | 85.34591 |
| Mitomycin C | 1.435128 | 1.370193 |
| MK 886 | 116.7328 | 112.1339 |
| MK-4827 (Niraparib) | 29.27844 | 28.69123 |
| ML-7 | 76.66721 | 78.88234 |
| MLN8237 | 36.79074 | 39.05608 |
| Monastrol | 102.2751 | 103.7334 |
| Monensin | 3.45484 | 3.954553 |
| MST-312 | 7.363489 | 7.703489 |
| N1,N12-Diethylspermine (BESpm) | 32.22598 | 37.80245 |
| N-Acetyl-L-cysteine | 185.7785 | 139.0585 |
| Nalidixic acid | 102.1144 | 97.46302 |
| Nifedipine | 104.2683 | 106.4182 |
| Nigericin | 3.466672 | 4.007355 |
| nilotinib | 85.8056 | 91.64786 |
| NL-71-101 | 242.279 | 157.1592 |
| Nocodazole | 6.511129 | 7.149245 |
| Nordihydroguaiaretic acid (NDGA) | 101.2347 | 112.0774 |
| N-phenylanthranilic acid | 101.4103 | 118.0042 |
| NS-398 | 176.8373 | 141.9483 |
| NSC625987 | 136.0173 | 161.7582 |
| NSC95397 | 41.92503 | 15.8033 |
| NU1025 | 174.885 | 123.2356 |

TABLE S1-9

| Drug | WT | KO |
|---|---|---|
| NU6102 | 112.51 | 124.8924 |
| Nutlin-3 | 23.89335 | 25.82262 |
| OBAA | 199.8448 | 110.9715 |
| ODQ | 84.47425 | 84.90051 |
| Olaparib | 20.86449 | 19.50809 |
| Oligomycin | 13.59351 | 21.69165 |
| Olomoucine | 173.0121 | 122.1855 |
| orlistat | 84.6498 | 86.10028 |
| OSI-906 | 59.0669 | 79.10312 |
| OSU-03012 | 91.92396 | 104.5247 |
| Ouabain | 4.809432 | 5.122407 |
| PAC-1 | 6.045455 | 9.440379 |
| Paclitaxel | 4.905022 | 6.42326 |
| pazopanib | 19.76101 | 39.75864 |
| PCI-34051 | 71.67381 | 72.30012 |
| PD 98059 | 168.8128 | 111.8217 |
| PD169316 | 161.5329 | 151.5532 |
| PD173074 | 8.446255 | 7.567502 |
| PDGF receptor tyrosine | 5.928361 | 12.88559 |
| kinase inhibitor IV | | |
| PDGF receptor tyrosine | 99.00466 | 107.0846 |
| kinase inhibitor V | | |
| Pepstatin A | 115.9467 | 126.4531 |
| PF-04217903 | 86.5829 | 93.9615 |
| PGP-4008 | 106.0362 | 117.1066 |
| Phenelzine | 101.3257 | 119.4827 |
| Pifithrin-a (cyclic) | 91.14888 | 99.94099 |
| PIM1 Inhibitor II | 97.56318 | 94.92055 |
| PIM½ Kinase Inhibitor V | 81.53059 | 78.31202 |

TABLE S1-10

| Drug | WT | KO |
|---|---|---|
| PJ-34 | 18.28302 | 15.93636 |
| PKR inhibitor | 8.714128 | 16.83675 |
| PP1 analog | 76.54529 | 69.2339 |
| PP2 | 121.2434 | 149.4142 |
| PP-H | 204.3806 | 121.6707 |
| PRIMA-1 | 84.24761 | 12.95386 |
| Purvalanol A | 51.31963 | 33.46943 |
| R59022 | 87.88597 | 103.798 |
| Radicicol | 7.121073 | 5.648444 |
| RAF1 kinase inhibitor I | 127.271 | 164.7652 |
| Rapamycin | 169.0383 | 125.2736 |
| RHC80267 | 82.45446 | 92.21762 |
| Ro 5-4864 | 92.87551 | 98.26561 |
| Ro-20-1724 | 163.0432 | 105.8008 |
| Rotenone | 5.524326 | 6.986626 |
| Rp-8-CPT-cOMPS | 110.9572 | 135.7084 |
| RS 102895 | 37.33646 | 47.57515 |
| Ruxolitinib | 58.12979 | 86.1151 |
| S2101 (LSD1 inhibitor II) | 80.51515 | 83.94889 |
| Sanguinarine | 1.826543 | 2.090956 |
| SB 203580 | 163.1226 | 176.0802 |
| SB 218078 | 101.994 | 109.2807 |
| SB 225002 | 5.2146 | 8.05633 |
| SB 328437 | 104.1947 | 112.6489 |
| SB 431542 | 85.03928 | 85.01854 |
| SB202190 | 108.8022 | 191.0939 |
| SB239063 | 118.4502 | 174.3691 |

TABLE S1-11

| Drug | WT | KO |
|---|---|---|
| Scriptaid | 6.951308 | 5.744929 |
| SC-αασ9 | 143.2439 | 153.4287 |
| SD208 | 73.72213 | 78.90325 |
| SIRT1 inhibitor III | 92.15094 | 98.93798 |
| Sodium salicylate | 176.7645 | 124.9147 |
| sorafenib | 61.42396 | 70.43826 |
| SP600125 | 172.059 | 104.9078 |
| Staurosporine | 5.221514 | 4.139408 |
| SU11274 | 71.93838 | 82.97645 |
| SU11652 | 4.73536 | 8.986771 |
| SU1498 | 165.2374 | 119.3751 |
| SU4984 | 129.369 | 172.2779 |
| SU5402 | 110.6763 | 139.2404 |
| SU6656 | 124.1846 | 170.9958 |
| Sulindac sulfide | 194.8834 | 140.1045 |
| sunitinib malate | 3.229274 | 5.953005 |
| Swainsonine | 115.0201 | 109.5472 |
| Syk inhibitor | 58.61585 | 91.09173 |
| tamibarotene | 77.33391 | 101.0643 |
| Tamoxifen, citrate | 24.80098 | 18.66146 |
| TBB | 187.1148 | 147.5332 |
| t-Butylhydroquinone (BHQ) | 95.36541 | 96.63559 |
| temozolomide | 84.41309 | 103.5311 |
| temsirolimus | 82.10577 | 74.95611 |
| Tenovin-6 | 2.513722 | 5.579114 |
| Terreic acid | 88.8751 | 90.53852 |
| TG003 | 115.1966 | 138.6968 |

TABLE S1-12

| Drug | WT | KO |
|---|---|---|
| TGF-b RI kinase inhibitor II | 90.90499 | 104.5326 |
| thalidomide | 94.96055 | 106.5408 |
| Thapsigargin | 2.628991 | 2.882364 |
| Theophylline | 202.6017 | 139.8574 |
| Thiazovivin | 50.22356 | 30.48697 |
| TMPyP4 | 19.34848 | 32.36555 |
| TOFA | 75.72252 | 74.56128 |
| Torkinib | 8.777015 | 9.987917 |
| Tpl2 kinase inhibitor | 89.04275 | 128.7549 |
| tretinoin | 77.73728 | 96.26223 |
| Trichostatin A | 6.35167 | 4.999529 |
| TrkA inhibitor | 58.6388 | 71.06629 |
| Troglitazone | 100.9481 | 94.73907 |
| Tunicamycin | 4.6969 | 6.010722 |
| TWS119 | 30.07318 | 23.39367 |
| TX-1918 | 117.0659 | 173.7673 |
| U0126 | 80.53573 | 68.28611 |
| UNC0638 | 2.602916 | 3.80009 |
| Valeryl salicylate | 169.5428 | 118.3391 |
| Valinomycin | 3.647564 | 4.172594 |
| Vandetanib | 14.13208 | 28.94087 |
| VEGF recptor 2 | 106.2493 | 109.4563 |
| kinase inhibitor I | | |
| VEGFR receptor tyrosine | 96.94069 | 131.6347 |
| kinase inhibitor II | | |
| Vemurafenib | 102.8042 | 107.9076 |
| Verapamil | 73.75689 | 88.19349 |
| Vinblastine sulfate | 2.725349 | 4.491811 |
| Vismodegib | 88.6761 | 108.8423 |

TABLE S1-13

| Drug | WT | KO |
|---|---|---|
| vorinostat | 2.818182 | 6.02102 |
| Wortmannin | 180.603 | 115.5187 |
| WP1066 | 15.29302 | 19.99027 |
| Xanthohumol | 103.677 | 112.4513 |
| XAV939 | 86.97713 | 110.6783 |
| Y27632 | 160.0262 | 98.56744 |
| YM155 | 2.670955 | 3.239632 |
| Zaprinast | 93.46947 | 82.56519 |
| Z-GLF-CMK | 4.254659 | 4.8907 |
| ZM 336372 | 121.6939 | 179.4192 |
| Z-VAD-FMK | 177.3841 | 128.7264 |

### INDUSTRIAL APPLICABILITY

It is possible to treat SWI/SNF complex-deficient cancer by administering a glutathione (GSH) metabolic pathway inhibitor, according to the disclosure of the present invention.

1. A method for treating SWI/SNF complex-deficient cancer, comprising administering an effective amount of a glutathione (GSH) metabolic pathway inhibitor to a mammal in need thereof.

2. The method according to claim 1, wherein the GSH metabolic pathway inhibitor is glutamate-cysteine ligase synthetase (GCL) inhibitor, glutamate-cysteine ligase catalytic subunit (GCLC) inhibitor, glutamate-cysteine ligase modifier (GCLM) inhibitor, GSH synthetase (GSS) inhibitor, SLC7A11 inhibitor, or a combination thereof.

3. The method according to claim 1, wherein the GSH metabolic pathway inhibitor is antibody, siRNA, shRNA, miRNA, antisense oligonucleotide, or low-molecular compound.

4. The method according to claim 1, wherein the SWI/SNF complex is ARID1A, SMARCA2 (BRM), SMARCA4 (BRG1), SMARCB1, PBRM1, or a combination thereof.

5. The method according to claim 1, wherein the SWI/SNF complex-deficient cancer is ARID1A-deficient cancer, and the ARID1A-deficient cancer is ovarian cancer, uterine cancer, gastric cancer, bladder cancer, bile duct cancer, liver cancer, esophageal cancer, lung cancer, colon cancer, pancreatic cancer, breast cancer, neuroblastoma, glioma, skin cancer, B-cell lymphoma or renal cancer.

6. The method according to claim 1, wherein the SWI/SNF complex-deficient cancer is SMARCA4 (BRG1)-deficient cancer, and the SMARCA4 (BRG1)-deficient cancer is bladder cancer, breast cancer, cervical cancer, colon cancer, B-cell lymphoma, esophageal cancer, gastric cancer, glioma, head and neck cancer, liver cancer, lung cancer, melanoma, pancreatic cancer, prostate cancer, renal cancer, rhabdoid tumor, ovarian cancer, SMARCA4-deficient thoracic sarcoma, or uterine cancer.

7. The method according to claim 1, wherein the SWI/SNF complex-deficient cancer is SMARCB1-deficient cancer, and the SMARCB1-deficient cancer is epithelioid sarcoma, familial schwannomatosis, gastric cancer, renal cancer, rhabdoid tumor, sinonasal carcinoma, synovial sarcoma, undifferentiated chordomas or uterine cancer.

8. The method according to claim 1, wherein the SWI/SNF complex-deficient cancer is PBRM1-deficient cancer which is bladder cancer, bile duct cancer, colon cancer, B-cell lymphoma, esophageal cancer, gastric cancer, head and neck cancer, lung cancer, melanoma, mesothelioma, pancreatic cancer, renal cancer, or uterine cancer.

9. The method according to claim 1, wherein the GSH metabolic pathway inhibitor is GSH reduction compound.

10. The method according to claim 10, wherein the GSH reduction compound is APR-017 or APR-246.

11. The method according to claim 2, wherein the GCLC inhibitor is buthionine sulfoximine (BSO).

12. The method according to claim 2, wherein the SLC7A11 inhibitor is sulfasalazine.

13. A method for detecting and/or selecting a susceptible patient to a glutathione (GSH) metabolic pathway inhibitor, comprising detecting the presence of SWI/SNF complex deficiency in a patient having cancer.

14. A pharmaceutical composition comprising an effective amount of a glutathione (GSH) metabolic pathway inhibitor for treating SWI/SNF complex-deficient cancer.

15. Glutathione (GSH) metabolic pathway inhibitor for use in treating SWI/SNF complex-deficient cancer.

16. A kit comprising (a) a glutathione (GSH) metabolic pathway inhibitor, and (b) instructions for treating SWI/SNF complex-deficient cancer.
